SEQUENCE LISTING (64 sequences)

SEQ ID NO: 1 - DNA, 10186 bp, synthetic construct
  misc_feature 1..10186: note = Synthetic: Modified Lentiviral Transfer Plasmid (complete)
  source 1..10186: mol_type = other DNA
tccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggttcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccctcgagccaccatgcagctggaactgtctacctgtgtgtttctgtgtctgctgcctctggggttttctgctatcaggagatactatctgggagctgtggagctgtcctgggactacaggcagtctgagctgctgagagaactgcatgtggataccagattcccagctacagctccaggagctctgcctctgggcccatctgtgctgtacaagaaaacagtctttgtggagtttacagaccagctgttctctgtggccaggccaagaccaccttggatgggactgctgggaccaaccatccaggctgaggtgtatgatacagtggtggtgaccctgaaaaacatggcctcccatcctgtgagcctgcatgctgtgggggtgtccttctggaagtcctctgagggagctgagtatgaagaccatacctcccagagggagaaagaagatgataaggtgctgcctggcaaaagccagacctatgtctggcaggtgctgaaggagaatggaccaactgcttctgacccaccatgcctgacctactcttatctgtcccatgtggatctggtgaaggacctgaattctggactgattggagctctgctggtgtgtagagagggaagcctgaccagagaaagaacccagaacctgcatgagtttgtcctgctgtttgctgtgtttgatgaagggaagagctggcactctgccaggaatgactcctggaccagagctatggatccagctcctgctagagctcagcctgctatgcacacagtcaatggctatgtgaataggtctctgccaggactgattggctgccataagaaatctgtctattggcatgtgattggaatgggcaccagccctgaggtgcattctatcttcctggaaggccacacctttctggtcaggcaccatagacaggcctctctggagatctcccctctgaccttcctgacagctcagacctttctgatggacctggggcagttcctgctgttttgccatatctcttcccaccatcatggaggaatggaggctcatgtcagggtggaatcctgtgctgaggaaccacagctgagaagaaaggctgatgaggaagaggactatgatgataacctgtatgactctgatatggatgtggtgaggctggatggggatgatgtcagccctttcatccagatcaggtctgtggccaagaaacatccaaagacctgggtccactacattgctgctgaagaggaagattgggactatgcccccctggtgctggctcctgatgatagatcctacaaaagccagtatctgaacaatgggccccagaggattggaaggaagtacaagaaagtgaggttcatggcctatacagatgagacctttaagaccagagaggctatccagcatgaatctgggatcctgggacctctgctgtatggagaagtgggggataccctgctgatcatcttcaagaaccaggcctccaggccatacaatatctatccccatggcatcacagatgtgagaccactgtacagcaggagactgcccaagggggtcaaacacctgaaggatttccccatcctgcctggagagatctttaagtataaatggacagtcacagtggaagatgggcctaccaagtctgatccaaggtgcctgaccagatactatagctcttttgtgaacatggagagagacctggcttctggactgattggacccctgctgatctgttacaaagagtctgtggaccagaggggcaaccagatcatgtctgataagagaaatgtcatcctgttctctgtgtttgatgagaacaggagctggtacctgacagagaacatccagaggttcctgccaaatccagctggagtgcagctggaggacccagaatttcaggcttccaacatcatgcatagcatcaatggctatgtgtttgatagcctgcagctgtctgtctgcctgcatgaggtggcctactggtatatcctgtccattggagctcagacagacttcctgtctgtgttctttagtgggtacacctttaagcataaaatggtgtatgaggataccctgaccctgttccccttttctggggagacagtgttcatgtccatggaaaaccctggcctgtggatcctggggtgccacaactctgacttcaggaatagaggaatgacagccctgctgaaagtgtccagctgtgataagaatacaggggattactatgaggactcttatgaagatatctctgcttatctgctgagcaagaacaatgccattgagcccaggtcttttgctcagaactccagacctccatctgcttctgctcctaagccacctgtgctgagaagacatcagagggacatctccctgcctaccttccagccagaggaagataaaatggactatgatgatatcttcagcacagagaccaagggggaagattttgacatctatggagaggatgaaaaccaggatccaagatccttccagaagagaaccagacactactttattgctgctgtggagcagctgtgggactatgggatgtctgaaagcccaagggccctgaggaacagagctcagaatggagaggtgcccagattcaagaaagtggtgttcagagagtttgctgatggcagctttacccagccatcttacaggggggagctgaacaagcatctggggctgctgggaccctatatcagagctgaggtggaagataacatcatggtgaccttcaagaatcaggcttctaggccctactccttttattcttccctgatctcctaccctgatgatcaggagcagggagctgaacctaggcacaactttgtgcagccaaatgagaccagaacctacttttggaaggtgcagcatcacatggctcccacagaggatgaatttgactgcaaagcttgggcctatttttctgatgtggacctggagaaggatgtgcattctggcctgattgggcctctgctgatctgtagggccaacaccctgaatgctgctcatggaagacaggtcacagtgcaggagtttgctctgttctttaccatctttgatgaaaccaagagctggtacttcacagagaatgtggaaaggaattgcagagccccctgtcatctgcagatggaggaccctaccctgaaggaaaactacaggttccatgccatcaatggatatgtcatggataccctgcctggcctggtcatggctcagaaccagaggatcagatggtacctgctgtctatgggatccaatgagaatatccatagcatccacttctctggccatgtcttttctgtgaggaagaaagaggaatacaaaatggctgtgtacaatctgtatcctggggtctttgagacagtggaaatgctgccaagcaaagtgggaatctggagaattgagtgcctgattggggaacacctgcaggctgggatgagcaccaccttcctggtgtactctaagaaatgtcagaccccactggggatggcctctggacatatcagggacttccagatcacagcttctggacagtatggacagtgggctccaaagctggctagactgcactattctggctccatcaatgcctggtctaccaaagagccattctcctggatcaaggtggacctgctggcccccatgatcatccatggaatcaaaacccagggagctaggcagaagttcagctctctgtacatctcccagtttatcatcatgtatagcctggatgggaagaaatggcagacctacagaggcaattccactgggaccctgatggtcttctttggaaatgtggattcctctggcatcaagcacaacatcttcaatccacccatcattgccaggtacatcaggctgcatcctacccactatagcatcaggtctaccctgagaatggagctgatgggatgtgacctgaacagctgttctatgccactgggcatggagtccaaggctatctctgatgcccagatcacagcttcttcctacttcaccaatatgtttgctacctggtccccaagcaaggctagactgcacctgcagggaagatccaatgcttggagaccccaggtgaacaatcctaaggagtggctgcaggtggacttccagaaaaccatgaaggtcacaggggtgaccacccagggagtgaaatctctgctgacctccatgtatgtcaaggagttcctgatcagctcttcccaggatggccaccagtggaccctgttctttcagaatggcaaggtcaaagtgttccaggggaatcaggactcttttaccccagtggtgaactccctggatcctccactgctgaccaggtacctgagaatccatcctcagagctgggtgcaccagattgctctgagaatggaggtcctgggatgtgaagctcaggacctgtattgagcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctta

SEQ ID NO: 2 - DNA, 659 bp, synthetic construct
  misc_feature 1..659: note = Synthetic: CD68 promoter
  source 1..659: mol_type = other DNA
tcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcc

SEQ ID NO: 3 - DNA, 4404 bp, synthetic construct
  misc_feature 1..4404: note = Synthetic: ET3 LCO
  source 1..4404: mol_type = other DNA
atgcagctggaactgtctacctgtgtgtttctgtgtctgctgcctctggggttttctgctatcaggagatactatctgggagctgtggagctgtcctgggactacaggcagtctgagctgctgagagaactgcatgtggataccagattcccagctacagctccaggagctctgcctctgggcccatctgtgctgtacaagaaaacagtctttgtggagtttacagaccagctgttctctgtggccaggccaagaccaccttggatgggactgctgggaccaaccatccaggctgaggtgtatgatacagtggtggtgaccctgaaaaacatggcctcccatcctgtgagcctgcatgctgtgggggtgtccttctggaagtcctctgagggagctgagtatgaagaccatacctcccagagggagaaagaagatgataaggtgctgcctggcaaaagccagacctatgtctggcaggtgctgaaggagaatggaccaactgcttctgacccaccatgcctgacctactcttatctgtcccatgtggatctggtgaaggacctgaattctggactgattggagctctgctggtgtgtagagagggaagcctgaccagagaaagaacccagaacctgcatgagtttgtcctgctgtttgctgtgtttgatgaagggaagagctggcactctgccaggaatgactcctggaccagagctatggatccagctcctgctagagctcagcctgctatgcacacagtcaatggctatgtgaataggtctctgccaggactgattggctgccataagaaatctgtctattggcatgtgattggaatgggcaccagccctgaggtgcattctatcttcctggaaggccacacctttctggtcaggcaccatagacaggcctctctggagatctcccctctgaccttcctgacagctcagacctttctgatggacctggggcagttcctgctgttttgccatatctcttcccaccatcatggaggaatggaggctcatgtcagggtggaatcctgtgctgaggaaccacagctgagaagaaaggctgatgaggaagaggactatgatgataacctgtatgactctgatatggatgtggtgaggctggatggggatgatgtcagccctttcatccagatcaggtctgtggccaagaaacatccaaagacctgggtccactacattgctgctgaagaggaagattgggactatgcccccctggtgctggctcctgatgatagatcctacaaaagccagtatctgaacaatgggccccagaggattggaaggaagtacaagaaagtgaggttcatggcctatacagatgagacctttaagaccagagaggctatccagcatgaatctgggatcctgggacctctgctgtatggagaagtgggggataccctgctgatcatcttcaagaaccaggcctccaggccatacaatatctatccccatggcatcacagatgtgagaccactgtacagcaggagactgcccaagggggtcaaacacctgaaggatttccccatcctgcctggagagatctttaagtataaatggacagtcacagtggaagatgggcctaccaagtctgatccaaggtgcctgaccagatactatagctcttttgtgaacatggagagagacctggcttctggactgattggacccctgctgatctgttacaaagagtctgtggaccagaggggcaaccagatcatgtctgataagagaaatgtcatcctgttctctgtgtttgatgagaacaggagctggtacctgacagagaacatccagaggttcctgccaaatccagctggagtgcagctggaggacccagaatttcaggcttccaacatcatgcatagcatcaatggctatgtgtttgatagcctgcagctgtctgtctgcctgcatgaggtggcctactggtatatcctgtccattggagctcagacagacttcctgtctgtgttctttagtgggtacacctttaagcataaaatggtgtatgaggataccctgaccctgttccccttttctggggagacagtgttcatgtccatggaaaaccctggcctgtggatcctggggtgccacaactctgacttcaggaatagaggaatgacagccctgctgaaagtgtccagctgtgataagaatacaggggattactatgaggactcttatgaagatatctctgcttatctgctgagcaagaacaatgccattgagcccaggtcttttgctcagaactccagacctccatctgcttctgctcctaagccacctgtgctgagaagacatcagagggacatctccctgcctaccttccagccagaggaagataaaatggactatgatgatatcttcagcacagagaccaagggggaagattttgacatctatggagaggatgaaaaccaggatccaagatccttccagaagagaaccagacactactttattgctgctgtggagcagctgtgggactatgggatgtctgaaagcccaagggccctgaggaacagagctcagaatggagaggtgcccagattcaagaaagtggtgttcagagagtttgctgatggcagctttacccagccatcttacaggggggagctgaacaagcatctggggctgctgggaccctatatcagagctgaggtggaagataacatcatggtgaccttcaagaatcaggcttctaggccctactccttttattcttccctgatctcctaccctgatgatcaggagcagggagctgaacctaggcacaactttgtgcagccaaatgagaccagaacctacttttggaaggtgcagcatcacatggctcccacagaggatgaatttgactgcaaagcttgggcctatttttctgatgtggacctggagaaggatgtgcattctggcctgattgggcctctgctgatctgtagggccaacaccctgaatgctgctcatggaagacaggtcacagtgcaggagtttgctctgttctttaccatctttgatgaaaccaagagctggtacttcacagagaatgtggaaaggaattgcagagccccctgtcatctgcagatggaggaccctaccctgaaggaaaactacaggttccatgccatcaatggatatgtcatggataccctgcctggcctggtcatggctcagaaccagaggatcagatggtacctgctgtctatgggatccaatgagaatatccatagcatccacttctctggccatgtcttttctgtgaggaagaaagaggaatacaaaatggctgtgtacaatctgtatcctggggtctttgagacagtggaaatgctgccaagcaaagtgggaatctggagaattgagtgcctgattggggaacacctgcaggctgggatgagcaccaccttcctggtgtactctaagaaatgtcagaccccactggggatggcctctggacatatcagggacttccagatcacagcttctggacagtatggacagtgggctccaaagctggctagactgcactattctggctccatcaatgcctggtctaccaaagagccattctcctggatcaaggtggacctgctggcccccatgatcatccatggaatcaaaacccagggagctaggcagaagttcagctctctgtacatctcccagtttatcatcatgtatagcctggatgggaagaaatggcagacctacagaggcaattccactgggaccctgatggtcttctttggaaatgtggattcctctggcatcaagcacaacatcttcaatccacccatcattgccaggtacatcaggctgcatcctacccactatagcatcaggtctaccctgagaatggagctgatgggatgtgacctgaacagctgttctatgccactgggcatggagtccaaggctatctctgatgcccagatcacagcttcttcctacttcaccaatatgtttgctacctggtccccaagcaaggctagactgcacctgcagggaagatccaatgcttggagaccccaggtgaacaatcctaaggagtggctgcaggtggacttccagaaaaccatgaaggtcacaggggtgaccacccagggagtgaaatctctgctgacctccatgtatgtcaaggagttcctgatcagctcttcccaggatggccaccagtggaccctgttctttcagaatggcaaggtcaaagtgttccaggggaatcaggactcttttaccccagtggtgaactccctggatcctccactgctgaccaggtacctgagaatccatcctcagagctgggtgcaccagattgctctgagaatggaggtcctgggatgtgaagctcaggacctgtattga

SEQ ID NO: 4 - protein, 1467 aa, synthetic construct
  REGION 1..1467: note = Synthetic: ET3 LCO Amino Acid Seq, synthetic fVIII
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - DNA, 8775 bp, synthetic construct
  misc_feature 1..8775: note = Synthetic: Complete Sequence of plasmid
  source 1..8775: mol_type = other DNA
tcagaaaaactcgtcgagcatcagatgaaactgcaacttattcatatcggggttatcaattccatatttttggaaaagcctcttctgtaaactaggagaaaactcgccgaggcagttccataggatggccagatcctggtatctgtctgcgattccaacccttcccacatcaatacaacctataagtttgccctcgtcaaaaatcaggttatcaagagagaaatcaccgtgagtgacgactgagtctggactgaatggcaaaagtttgtgcatttctttccagacctgttccacgggccagccatttctctcgtcatcgaaatcactagcatccaccaaaccgttattcattcgtgactgggcctgagcgagacgaaatacgcgatcgctgttaaaagggcaattacaaacaggaatggaatgcaaccgtctcaggaacactgccagagcgtcaacaatattttcgcctgaatcagggtattcttccagtacctggaatgctgtctttccggggatcgcagtggtgagtaaccatgcgtcatctggagtacggataaaatgcttgatggttggaagaggcataaattcggtcagccagtttagtctcaccatctcatctgtcacatcattggcaacgctaccttttccatgcttcaggaacagctctggagcatcgggcttcccatacaatcggtagattgtggctccagattgcccgacattgtcgcgagcccatttatacccatataagtcagcgtccatgttggaattgagtctcggcctggacgtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcttatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattaagtagttgattaactaattaggtgattcacctaattagttaatcaactacgtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttgcggccgcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagtttaaacggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 6 - DNA, 47 bp, synthetic construct
  misc_feature 1..47: note = Synthetic: Synthetic nucleotide sequence, 6 frame stop element
  source 1..47: mol_type = other DNA
tagttgattaactaattaggtgattcacctaattagttaatcaacta

SEQ ID NO: 7 - DNA, 810 bp, synthetic construct
  misc_feature 1..810: note = Synthetic: hCD8 codon optimized Kanamycin resistance
  source 1..810: mol_type = other DNA
tcagaaaaactcgtcgagcatcagatgaaactgcaacttattcatatcggggttatcaattccatatttttggaaaagcctcttctgtaaactaggagaaaactcgccgaggcagttccataggatggccagatcctggtatctgtctgcgattccaacccttcccacatcaatacaacctataagtttgccctcgtcaaaaatcaggttatcaagagagaaatcaccgtgagtgacgactgagtctggactgaatggcaaaagtttgtgcatttctttccagacctgttccacgggccagccatttctctcgtcatcgaaatcactagcatccaccaaaccgttattcattcgtgactgggcctgagcgagacgaaatacgcgatcgctgttaaaagggcaattacaaacaggaatggaatgcaaccgtctcaggaacactgccagagcgtcaacaatattttcgcctgaatcagggtattcttccagtacctggaatgctgtctttccggggatcgcagtggtgagtaaccatgcgtcatctggagtacggataaaatgcttgatggttggaagaggcataaattcggtcagccagtttagtctcaccatctcatctgtcacatcattggcaacgctaccttttccatgcttcaggaacagctctggagcatcgggcttcccatacaatcggtagattgtggctccagattgcccgacattgtcgcgagcccatttatacccatataagtcagcgtccatgttggaattgagtctcggcctggacgtttcccgttgaatgtggctcat

SEQ ID NO: 8 - protein, 269 aa, synthetic construct
  REGION 1..269: note = Synthetic: hCD8 codon optimized Kanamycin resistance
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 9 - DNA, 1242 bp, synthetic construct
  misc_feature 1..1242: note = Synthetic: CMV promoter + beta-globin intron
  source 1..1242: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcttatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 10 - DNA, 462 bp, synthetic construct
  misc_feature 1..462: note = Synthetic: PGK polyA signal
  source 1..462: mol_type = other DNA
ctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaa

SEQ ID NO: 11 - DNA, 1503 bp, synthetic construct
  misc_feature 1..1503: note = Synthetic: Gag
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 12 - protein, 500 aa, synthetic construct
  REGION 1..500: note = Synthetic: Gag
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 13 - DNA, 3012 bp, synthetic construct
  misc_feature 1..3012: note = Synthetic: Pol
  source 1..3012: mol_type = other DNA
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattaa

SEQ ID NO: 14 - protein, 1003 aa, synthetic construct
  REGION 1..1003: note = Synthetic: Pol AA
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 15 - DNA, 5531 bp, synthetic construct
  misc_feature 1..5531: note = Synthetic: VSVG Plasmid, Complete Sequence
  source 1..5531: mol_type = other DNA
tcagaaaaactcatcgagcatcagatggaactgcaatttattcatgtcagggttatctatgccgtatttttggaaaagccgcttctgcagtgagggagaaaactcaccgaggcagttccagaggatggcaagatcctggtacctgtcggcgattccgactcgtcccacgtcaatgcagcctatgagtttcccctcgtcgaaaataaggttatccagtgagaaatccccgtgagtcacgaccgaatccggactgaatggcaacagcttgtgcatttccttccacacctgctccacaggccagccatttctctcgtcgtcaaaatcgctggcatcaaccagaccgttgttcattctggattgagcctgagcgagacgaaatacgcggtcgctgttaaatggacagttacacacgggaatagagtgcaatctgcgcaggaacactgccagagcgtcaacaatattttcgccactatcgggatattcttctagtacctggaatgctgtctttccggggatcgcagtggtgagtaaccatgcgtcatcaggggtacggatgaaatgcttgatggtcggcagaggcataaattcggtcagccagtttagtctgaccatctcatctgtcacatcattggcaacgctgccttttccgtgcttcagaaacagctctggggcgtcgggcttcccgtacaacctatagattgtggcgccggactgcccgacattgtcccgagcccatttatacccgtacaggtcagcgtccatgttggaatttaatcgtggcctggacgtctcccgctgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgagccaccatgaagtgtttgctgtaccttgcgttcctgtttattggggtgaactgcaagttcactatcgtgtttcctcacaaccaaaaaggcaactggaaaaacgtgccttcaaattatcattattgcccctctagtagcgacctgaactggcataatgacttgattgggaccgctctgcaggtgaagatgccaaagagccacaaggctatccaggctgatggttggatgtgtcatgcctctaaatgggtaactacctgtgatttccgctggtatggaccaaaatatataacacacagcatcaggagtttcacaccgagtgttgaacaatgtaaagaaagtatcgaacagactaagcagggtacctggctgaaccctggttttcccccacagtcatgtgggtatgcaaccgtgacagatgctgaggccgtgattgtgcaggtcacgccacatcatgtgcttgttgatgagtacaccggagagtgggtcgattcacagttcataaatggcaagtgctccaactacatttgcccaaccgtgcacaacagcactacttggcacagtgactacaaagttaagggtttatgtgattccaacctgatctccatggatatcacattcttttctgaagacggcgagctgtcctctctaggcaaagaaggaactgggtttcgaagtaattattttgcctacgaaacaggaggaaaagcctgcaagatgcagtattgtaaacactggggcgtgagactacccagcggtgtttggttcgagatggccgataaggacctgtttgcagcagcaagattcccggaatgccctgagggatccagcatttccgctccatcacagacttctgtcgacgttagcctgatccaggatgtggaacgcattctcgactattcgctttgccaggagacctggtcaaagataagagctggacttcccatctcacccgtcgatttatcttacctggccccaaagaatcccggcaccggaccagcgttcaccataatcaatggcacgctcaaatactttgagacgcgttacattcgggtagacattgcagctcctattctctctcggatggtaggtatgatctctgggactacaacagaacgcgagctctgggacgactgggcaccttacgaggatgtcgagatcggacctaacggagtgctccggacctcttcagggtataagtttcctctctatatgatcggccacgggatgttggacagcgatctgcatctgtcttccaaagcccaggtgtttgaacacccacacattcaagacgccgccagccagctgccagatgatgagtcgcttttctttggtgacacaggcttgagtaagaatcccattgagctggtagaaggatggttttcatcctggaaaagttcaatagcttcctttttcttcattattggcttaattatcggcttgtttttggtcctacgtgttggtattcatctttgtatcaaactcaagcatactaagaagcgacaaatctacacagatatcgagatgaataggctgggcaagtgagcggccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcctaggggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 16 - DNA, 810 bp, synthetic construct
  misc_feature 1..810: note = Synthetic: Human NK cell codon optimized kanamycin resistance
  source 1..810: mol_type = other DNA
tcagaaaaactcatcgagcatcagatggaactgcaatttattcatgtcagggttatctatgccgtatttttggaaaagccgcttctgcagtgagggagaaaactcaccgaggcagttccagaggatggcaagatcctggtacctgtcggcgattccgactcgtcccacgtcaatgcagcctatgagtttcccctcgtcgaaaataaggttatccagtgagaaatccccgtgagtcacgaccgaatccggactgaatggcaacagcttgtgcatttccttccacacctgctccacaggccagccatttctctcgtcgtcaaaatcgctggcatcaaccagaccgttgttcattctggattgagcctgagcgagacgaaatacgcggtcgctgttaaatggacagttacacacgggaatagagtgcaatctgcgcaggaacactgccagagcgtcaacaatattttcgccactatcgggatattcttctagtacctggaatgctgtctttccggggatcgcagtggtgagtaaccatgcgtcatcaggggtacggatgaaatgcttgatggtcggcagaggcataaattcggtcagccagtttagtctgaccatctcatctgtcacatcattggcaacgctgccttttccgtgcttcagaaacagctctggggcgtcgggcttcccgtacaacctatagattgtggcgccggactgcccgacattgtcccgagcccatttatacccgtacaggtcagcgtccatgttggaatttaatcgtggcctggacgtctcccgctgaatatggctcat

SEQ ID NO: 17 - protein, 511 aa, synthetic construct
  REGION 1..511: note = Synthetic: Amino Acid of SEQ ID NO: 16
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - DNA, 1267 bp, synthetic construct
  misc_feature 1..1267: note = Synthetic: PGK promoter/PGK intron
  source 1..1267: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacag

SEQ ID NO: 19 - DNA, 1536 bp, synthetic construct
  misc_feature 1..1536: note = Synthetic: 293T codon optimized VSVG coding sequence
  source 1..1536: mol_type = other DNA
atgaagtgtttgctgtaccttgcgttcctgtttattggggtgaactgcaagttcactatcgtgtttcctcacaaccaaaaaggcaactggaaaaacgtgccttcaaattatcattattgcccctctagtagcgacctgaactggcataatgacttgattgggaccgctctgcaggtgaagatgccaaagagccacaaggctatccaggctgatggttggatgtgtcatgcctctaaatgggtaactacctgtgatttccgctggtatggaccaaaatatataacacacagcatcaggagtttcacaccgagtgttgaacaatgtaaagaaagtatcgaacagactaagcagggtacctggctgaaccctggttttcccccacagtcatgtgggtatgcaaccgtgacagatgctgaggccgtgattgtgcaggtcacgccacatcatgtgcttgttgatgagtacaccggagagtgggtcgattcacagttcataaatggcaagtgctccaactacatttgcccaaccgtgcacaacagcactacttggcacagtgactacaaagttaagggtttatgtgattccaacctgatctccatggatatcacattcttttctgaagacggcgagctgtcctctctaggcaaagaaggaactgggtttcgaagtaattattttgcctacgaaacaggaggaaaagcctgcaagatgcagtattgtaaacactggggcgtgagactacccagcggtgtttggttcgagatggccgataaggacctgtttgcagcagcaagattcccggaatgccctgagggatccagcatttccgctccatcacagacttctgtcgacgttagcctgatccaggatgtggaacgcattctcgactattcgctttgccaggagacctggtcaaagataagagctggacttcccatctcacccgtcgatttatcttacctggccccaaagaatcccggcaccggaccagcgttcaccataatcaatggcacgctcaaatactttgagacgcgttacattcgggtagacattgcagctcctattctctctcggatggtaggtatgatctctgggactacaacagaacgcgagctctgggacgactgggcaccttacgaggatgtcgagatcggacctaacggagtgctccggacctcttcagggtataagtttcctctctatatgatcggccacgggatgttggacagcgatctgcatctgtcttccaaagcccaggtgtttgaacacccacacattcaagacgccgccagccagctgccagatgatgagtcgcttttctttggtgacacaggcttgagtaagaatcccattgagctggtagaaggatggttttcatcctggaaaagttcaatagcttcctttttcttcattattggcttaattatcggcttgtttttggtcctacgtgttggtattcatctttgtatcaaactcaagcatactaagaagcgacaaatctacacagatatcgagatgaataggctgggcaagtga

SEQ ID NO: 20 - protein, 511 aa, synthetic construct; identical to SEQ ID NO: 17
  REGION 1..511: note = Synthetic: VSVG AA

SEQ ID NO: 21 - DNA, 477 bp, synthetic construct
  misc_feature 1..477: note = Synthetic: Human growth hormone polyA
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 22 - DNA, 5127 bp, synthetic construct
  misc_feature 1..5127: note = Synthetic: Transfer Plasmid no Transgene
  source 1..5127: mol_type = other DNA
taatcccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtgcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagataagcttggtg

SEQ ID NO: 23 - DNA, 810 bp, synthetic construct
  misc_feature 1..810: note = Synthetic: Liver codon optimized kanamycin resistance gene
  source 1..810: mol_type = other DNA
tcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcat

SEQ ID NO: 24 - DNA, 41 bp, synthetic construct
  misc_feature 1..41: note = Synthetic: HIV antisense protein start codon disruption
  source 1..41: mol_type = other DNA
gcaactcacagtctggggcaacaagcagctccaggcaagaa

SEQ ID NO: 25 - DNA, 93 bp, synthetic construct
  misc_feature 1..93: note = Synthetic: 6 frame triple stop codon insulator
  source 1..93: mol_type = other DNA
tcagctacttattaattaggtgactagttgattaaaaccggtgcggccgccgccggtgtaattaggtgactagttgattaaatcagctactta

SEQ ID NO: 26 - DNA, 22 bp, synthetic construct
  misc_feature 1..22: note = Synthetic: Transgene insertion site
  source 1..22: mol_type = other DNA
accggtgcggccgccgccggtg

SEQ ID NO: 27 - DNA, 139 bp, synthetic construct
  misc_feature 1..139: note = Synthetic: Integrase stop codon
  source 1..139: mol_type = other DNA
ggcagtattcatccacaattttaaaagaaaaggggggattggggggtacagtgcaggggaaagaatagtagacataatagcaacagacatacaaactaaagaattacaaaaacaaattacaaaaattcaaaatttttaa

SEQ ID NO: 28 - DNA, 10729 bp, synthetic construct
  misc_feature 1..10729: note = Synthetic: Transfer plasmid
  source 1..10729: mol_type = other DNA
tccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtagcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagattcgagccaccatgcagctggagctctcaacctgtgtgttcctctgcctgctccccctgggattttcagctatcaggagatactatctgggagcagtggaactgtcctgggactacaggcagtcagagctgctcagagaactgcatgtggatactaggttccctgcaacagctcctggagcactgccactgggaccttcagtgctgtacaagaaaactgtctttgtggagtttacagaccagctgttcagtgtggccaggcccaggcccccctggatggggctgctgggacccaccatccaggctgaagtgtatgatactgtggtggtgaccctgaaaaacatggcctctcatccagtcagcctgcatgctgtgggagtgagcttctggaagagcagtgagggagctgagtatgaagaccatacctcacagagggagaaagaagatgataaggtgctgccaggaaaaagccagacctatgtgtggcaggtgctgaaggagaatggccctacagcttcagatcctccctgcctcacatactcttatctgagccatgtggatctggtgaaggacctcaatagtggcctgattggggcactgctggtgtgcagagaggggtccctcacaagggaaagaactcagaacctgcatgagtttgtcctgctctttgctgtgtttgatgagggaaagtcctggcactcagcaaggaatgacagctggaccagggctatggacccagcaccagccagagctcagccagctatgcacactgtcaatggctatgtgaataggtccctgcctggactcattggctgccataagaaatcagtctattggcatgtgattggaatgggcaccagcccagaggtgcattccatcttcctggaaggccacacatttctggtcaggcaccatagacaggccagcctggagatcagcccactgactttcctcacagcacagacatttctgatggacctggggcagttcctgctcttttgccatatctcaagtcaccatcatggagggatggaggctcatgtcagggtggaaagctgtgcagaggaacctcagctgaggaggaaggcagatgaggaagaggactatgatgataacctgtatgactcagatatggatgtggtgaggctggatggagatgatgtcagcccattcatccagatcaggtcagtggctaagaaacaccctaagacctgggtccactacattgcagctgaagaggaagattgggactatgcacccctggtgctggccccagatgatagaagttacaaatctcagtatctgaacaatgggccccagaggattggaaggaagtacaagaaagtgaggttcatggcttatactgatgagacctttaagacaagagaggcaatccagcatgaaagtggcatcctgggaccactgctctatggagaagtgggggataccctgctcatcatcttcaagaaccaggcctcaaggccttacaatatctatccccatggcatcacagatgtgaggcctctctacagcaggagactgcccaagggagtcaaacacctcaaggatttccccatcctgccaggggaaatcttcaagtataaatggacagtcactgtggaagatgggccaactaagtcagatcctaggtgcctgaccaggtactattctagctttgtgaacatggagagggacctggcttcaggactgattggacctctgctcatctgctacaaagaatcagtggaccagaggggcaaccagatcatgagtgataagagaaatgtcatcctgttctcagtgtttgatgagaataggagttggtatctgacagaaaacatccagaggttcctgcctaatcctgcaggagtgcagctggaggacccagaatttcaggcttcaaacatcatgcatagtatcaatggctatgtgtttgatagtctgcagctctctgtctgcctgcatgaggtggcctactggtatatcctcagcattggagctcagactgacttcctgagtgtgttcttttcaggctacacattcaagcataagatggtctatgaagataccctgacactcttccccttttctggggagactgtgtttatgagcatggaaaacccaggcctgtggattctggggtgccacaacagtgacttcaggaatagagggatgactgctctgctcaaagtgtcctcatgtgataagaatactggagattactatgaggactcttatgaagatatcagtgcatatctgctctccaaaaacaatgccattgagcccaggtcatttgctcagaacagtagaccaccttctgcaagtgcaccaaagcctccagtgctgaggagacaccagagggacatcagcctgccaaccttccagcctgaggaagataaaatggactatgatgatatcttctccactgagaccaagggggaagattttgacatctatggagaggatgaaaaccaggaccccaggtccttccagaagaggaccagacactactttattgcagctgtggagcagctgtgggactatggcatgtctgaatcacctagagctctgaggaacagagcacagaatggggaggtgcccaggttcaagaaagtggtgttcagagaatttgcagatggctcttttacccagcctagctacaggggggagctcaacaagcatctggggctgctgggaccctatatcagagcagaggtggaagataacatcatggtgacattcaagaatcaggcctcaagaccctacagtttttatagttctctgatcagctacccagatgatcaggagcagggggctgaaccaaggcacaactttgtgcagcctaatgagacaagaacttacttttggaaggtccagcatcacatggctcccacagaggatgagtttgactgcaaggcctgggcatatttttctgatgtggacctggagaaggatgtgcatagtggcctcattgggccactgctcatctgcagggcaaacacactgaatgctgcacatggcaggcaggtcactgtgcaggagtttgccctgttctttacaatctttgatgaaactaagtcctggtacttcacagagaatgtggaaaggaattgcagagccccctgccatctccagatggaggacccaactctgaaggaaaactacaggttccatgctatcaatggatatgtcatggatacactgccaggcctggtgatggcacagaaccagaggatcaggtggtatctgctcagcatggggtccaatgagaatatccattctatccacttctcaggacatgtcttttcagtgaggaagaaagaggaatataaaatggctgtgtacaatctgtatccaggggtctttgagacagtggaaatgctgcctagcaaagtggggatctggagaattgagtgcctcattggagaacacctgcaggcagggatgtccaccacatttctggtgtactcaaagaaatgccagactcccctggggatggcaagtggacatatcagggacttccagatcactgcatcaggacagtatggacagtgggcaccaaagctggctaggctccactatagtggctctatcaatgcttggagtaccaaagagcctttctcttggatcaaggtggatctgctggcccccatgatcatccatggaatcaaaacacagggagctagacagaagttcagctccctgtacatcagtcagtttatcatcatgtattctctggatgggaagaaatggcagacctacaggggcaatagcactgggacactgatggtcttctttggaaatgtggattcaagtggcatcaagcacaacatcttcaatcctcccatcattgccaggtacatcagactgcatcccacacactattcaatcaggagtactctcagaatggagctgatggggtgtgacctcaacagctgctccatgccactgggaatggaatccaaggcaatctcagatgcccagatcactgcttctagctacttcaccaatatgtttgcaacatggtcacccagtaaagcaaggctgcacctccagggaaggtccaatgcttggagaccccaggtgaacaatccaaaggagtggctgcaggtggactttcagaaaaccatgaaggtcacaggggtgactacccagggagtgaaaagtctgctcacctctatgtatgtcaaggagttcctgatctcctcaagtcaggatggccaccagtggacactgttctttcagaatggcaaggtcaaagtgttccaggggaatcaggacagctttacaccagtggtgaacagcctggacccccctctgctcactagatatctgagaatccatccacagagctgggtgcaccagattgcactcagaatggaggtcctgggctgtgaagcccaggacctgtattgagcggccccggtgaagggcgaattccagcacactggcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctta

SEQ ID NO: 29 - DNA, 1165 bp, synthetic construct
  misc_feature 1..1165: note = Synthetic: Modified EF1a promoter
  source 1..1165: mol_type = other DNA
gcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 30 - DNA, 4404 bp, synthetic construct
  misc_feature 1..4404: note = Synthetic: MCO ET3, Synthetic fVIII
  source 1..4404: mol_type = other DNA
atgcagctggagctctcaacctgtgtgttcctctgcctgctccccctgggattttcagctatcaggagatactatctgggagcagtggaactgtcctgggactacaggcagtcagagctgctcagagaactgcatgtggatactaggttccctgcaacagctcctggagcactgccactgggaccttcagtgctgtacaagaaaactgtctttgtggagtttacagaccagctgttcagtgtggccaggcccaggcccccctggatggggctgctgggacccaccatccaggctgaagtgtatgatactgtggtggtgaccctgaaaaacatggcctctcatccagtcagcctgcatgctgtgggagtgagcttctggaagagcagtgagggagctgagtatgaagaccatacctcacagagggagaaagaagatgataaggtgctgccaggaaaaagccagacctatgtgtggcaggtgctgaaggagaatggccctacagcttcagatcctccctgcctcacatactcttatctgagccatgtggatctggtgaaggacctcaatagtggcctgattggggcactgctggtgtgcagagaggggtccctcacaagggaaagaactcagaacctgcatgagtttgtcctgctctttgctgtgtttgatgagggaaagtcctggcactcagcaaggaatgacagctggaccagggctatggacccagcaccagccagagctcagccagctatgcacactgtcaatggctatgtgaataggtccctgcctggactcattggctgccataagaaatcagtctattggcatgtgattggaatgggcaccagcccagaggtgcattccatcttcctggaaggccacacatttctggtcaggcaccatagacaggccagcctggagatcagcccactgactttcctcacagcacagacatttctgatggacctggggcagttcctgctcttttgccatatctcaagtcaccatcatggagggatggaggctcatgtcagggtggaaagctgtgcagaggaacctcagctgaggaggaaggcagatgaggaagaggactatgatgataacctgtatgactcagatatggatgtggtgaggctggatggagatgatgtcagcccattcatccagatcaggtcagtggctaagaaacaccctaagacctgggtccactacattgcagctgaagaggaagattgggactatgcacccctggtgctggccccagatgatagaagttacaaatctcagtatctgaacaatgggccccagaggattggaaggaagtacaagaaagtgaggttcatggcttatactgatgagacctttaagacaagagaggcaatccagcatgaaagtggcatcctgggaccactgctctatggagaagtgggggataccctgctcatcatcttcaagaaccaggcctcaaggccttacaatatctatccccatggcatcacagatgtgaggcctctctacagcaggagactgcccaagggagtcaaacacctcaaggatttccccatcctgccaggggaaatcttcaagtataaatggacagtcactgtggaagatgggccaactaagtcagatcctaggtgcctgaccaggtactattctagctttgtgaacatggagagggacctggcttcaggactgattggacctctgctcatctgctacaaagaatcagtggaccagaggggcaaccagatcatgagtgataagagaaatgtcatcctgttctcagtgtttgatgagaataggagttggtatctgacagaaaacatccagaggttcctgcctaatcctgcaggagtgcagctggaggacccagaatttcaggcttcaaacatcatgcatagtatcaatggctatgtgtttgatagtctgcagctctctgtctgcctgcatgaggtggcctactggtatatcctcagcattggagctcagactgacttcctgagtgtgttcttttcaggctacacattcaagcataagatggtctatgaagataccctgacactcttccccttttctggggagactgtgtttatgagcatggaaaacccaggcctgtggattctggggtgccacaacagtgacttcaggaatagagggatgactgctctgctcaaagtgtcctcatgtgataagaatactggagattactatgaggactcttatgaagatatcagtgcatatctgctctccaaaaacaatgccattgagcccaggtcatttgctcagaacagtagaccaccttctgcaagtgcaccaaagcctccagtgctgaggagacaccagagggacatcagcctgccaaccttccagcctgaggaagataaaatggactatgatgatatcttctccactgagaccaagggggaagattttgacatctatggagaggatgaaaaccaggaccccaggtccttccagaagaggaccagacactactttattgcagctgtggagcagctgtgggactatggcatgtctgaatcacctagagctctgaggaacagagcacagaatggggaggtgcccaggttcaagaaagtggtgttcagagaatttgcagatggctcttttacccagcctagctacaggggggagctcaacaagcatctggggctgctgggaccctatatcagagcagaggtggaagataacatcatggtgacattcaagaatcaggcctcaagaccctacagtttttatagttctctgatcagctacccagatgatcaggagcagggggctgaaccaaggcacaactttgtgcagcctaatgagacaagaacttacttttggaaggtccagcatcacatggctcccacagaggatgagtttgactgcaaggcctgggcatatttttctgatgtggacctggagaaggatgtgcatagtggcctcattgggccactgctcatctgcagggcaaacacactgaatgctgcacatggcaggcaggtcactgtgcaggagtttgccctgttctttacaatctttgatgaaactaagtcctggtacttcacagagaatgtggaaaggaattgcagagccccctgccatctccagatggaggacccaactctgaaggaaaactacaggttccatgctatcaatggatatgtcatggatacactgccaggcctggtgatggcacagaaccagaggatcaggtggtatctgctcagcatggggtccaatgagaatatccattctatccacttctcaggacatgtcttttcagtgaggaagaaagaggaatataaaatggctgtgtacaatctgtatccaggggtctttgagacagtggaaatgctgcctagcaaagtggggatctggagaattgagtgcctcattggagaacacctgcaggcagggatgtccaccacatttctggtgtactcaaagaaatgccagactcccctggggatggcaagtggacatatcagggacttccagatcactgcatcaggacagtatggacagtgggcaccaaagctggctaggctccactatagtggctctatcaatgcttggagtaccaaagagcctttctcttggatcaaggtggatctgctggcccccatgatcatccatggaatcaaaacacagggagctagacagaagttcagctccctgtacatcagtcagtttatcatcatgtattctctggatgggaagaaatggcagacctacaggggcaatagcactgggacactgatggtcttctttggaaatgtggattcaagtggcatcaagcacaacatcttcaatcctcccatcattgccaggtacatcagactgcatcccacacactattcaatcaggagtactctcagaatggagctgatggggtgtgacctcaacagctgctccatgccactgggaatggaatccaaggcaatctcagatgcccagatcactgcttctagctacttcaccaatatgtttgcaacatggtcacccagtaaagcaaggctgcacctccagggaaggtccaatgcttggagaccccaggtgaacaatccaaaggagtggctgcaggtggactttcagaaaaccatgaaggtcacaggggtgactacccagggagtgaaaagtctgctcacctctatgtatgtcaaggagttcctgatctcctcaagtcaggatggccaccagtggacactgttctttcagaatggcaaggtcaaagtgttccaggggaatcaggacagctttacaccagtggtgaacagcctggacccccctctgctcactagatatctgagaatccatccacagagctgggtgcaccagattgcactcagaatggaggtcctgggctgtgaagcccaggacctgtattga

SEQ ID NO: 31 - protein, 1467 aa, synthetic construct; identical to SEQ ID NO: 4
  REGION 1..1467: note = Synthetic: MCO ET3, Synthetic fVIII

SEQ ID NO: 32 - DNA, 3494 bp, synthetic construct
  misc_feature 1..3494: note = Synthetic: plasmid
  source 1..3494: mol_type = other DNA
tcagaagaactcatcgagcatcaagtgaaactgtagcttgttcatatcaggattatcaattccgtatttctgaaatagccgtttctgcagtgatggagagaactcgccgaggcagttccagaggatggcaagatcctggtatcggtcagcgattcccactcgtcctacgtcaatacagcctattaatttcccctcgtcaaaaataaggttatcaagtgagaaatcgccgtgagtcacgacactgtcaggactgaatggcaaaagtttatgcatttccttccagacttgttccacaggccagccattccgctcgtcgtcgaagtcactggcgtcaaccagaccgttattcatcctagattgtgcctgagcgagacgaaatacgcggtcgctgttaaaggggcagttacacacaggaatggaatgcaaccggcgcaggaacactgccagggcatccacaatattttcacctgaatcaggatactcttccagtacctggaatgctgtctttccggggatggcagtggtgagtaaccatgcatcatctggagtacggataaagtgcttgatggtgggcagtggcataaattctgtcagccagttgagtctgaccatctcgtcggtaacgtcattggcaacgctaccctttccatgcttcagaaacaactctggagcatcgggcttgccatacaggcgatagattgtcgcaccggactgccccacattgtccctagcccatttgtacccatacagatcagcatccatgttggaattcagtctcggcctagacgtttctctttgtatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgtaagtttagtctttttgtcttttatttcaggtcccggatccggtggtggtgcaaatcaaagaactgctcctcagtggatgttgcctttacttctagctcgagccaccatggcaggcagatcaggggactccgatgaggatctccttaaggccgtgcgtctcataaagtttctgtatcagagtaatcctccacccaaccccgaaggtacacgccaagctcggaggaatagacgtagacgctggcgagaacggcagcgacaaatccacagcatttctgagcgcattctttcgacttacttggggcggtcagccgaacctgtcccgctgcagctacccccactggagaggttgactctggattgtaacgaggactgcggtacaagtggaacccagggcgttggaagcccacagatcctggtggagtctcctaccatcttagaatccggcgctaaagagtgagcggccgcgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaaagcttggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 33 - DNA, 810 bp, synthetic construct
  misc_feature 1..810: note = Synthetic: kanamycin resistance gene
  source 1..810: mol_type = other DNA
tcagaagaactcatcgagcatcaagtgaaactgtagcttgttcatatcaggattatcaattccgtatttctgaaatagccgtttctgcagtgatggagagaactcgccgaggcagttccagaggatggcaagatcctggtatcggtcagcgattcccactcgtcctacgtcaatacagcctattaatttcccctcgtcaaaaataaggttatcaagtgagaaatcgccgtgagtcacgacactgtcaggactgaatggcaaaagtttatgcatttccttccagacttgttccacaggccagccattccgctcgtcgtcgaagtcactggcgtcaaccagaccgttattcatcctagattgtgcctgagcgagacgaaatacgcggtcgctgttaaaggggcagttacacacaggaatggaatgcaaccggcgcaggaacactgccagggcatccacaatattttcacctgaatcaggatactcttccagtacctggaatgctgtctttccggggatggcagtggtgagtaaccatgcatcatctggagtacggataaagtgcttgatggtgggcagtggcataaattctgtcagccagttgagtctgaccatctcgtcggtaacgtcattggcaacgctaccctttccatgcttcagaaacaactctggagcatcgggcttgccatacaggcgatagattgtcgcaccggactgccccacattgtccctagcccatttgtacccatacagatcagcatccatgttggaattcagtctcggcctagacgtttctctttgtatgtggctcat

SEQ ID NO: 34 - protein, 269 aa, synthetic construct; identical to SEQ ID NO: 8
  REGION 1..269: note = Synthetic: kanamycin resist AA

SEQ ID NO: 35 - DNA, 497 bp, synthetic construct
  misc_feature 1..497: note = Synthetic: UbC promoter + SV40 intron
  source 1..497: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgtaagtttagtctttttgtcttttatttcaggtcccggatccggtggtggtgcaaatcaaagaactgctcctcagtggatgttgcctttacttctag

SEQ ID NO: 36 - DNA, 351 bp, synthetic construct
  misc_feature 1..351: note = Synthetic: 293T codon optimized rev
  source 1..351: mol_type = other DNA
atggcaggcagatcaggggactccgatgaggatctccttaaggccgtgcgtctcataaagtttctgtatcagagtaatcctccacccaaccccgaaggtacacgccaagctcggaggaatagacgtagacgctggcgagaacggcagcgacaaatccacagcatttctgagcgcattctttcgacttacttggggcggtcagccgaacctgtcccgctgcagctacccccactggagaggttgactctggattgtaacgaggactgcggtacaagtggaacccagggcgttggaagcccacagatcctggtggagtctcctaccatcttagaatccggcgctaaagagtga

SEQ ID NO: 37 - protein, 511 aa, synthetic construct; identical to SEQ ID NO: 17
  REGION 1..511: note = Synthetic: 293T codon optimized rev AA

SEQ ID NO: 38 - DNA, 395 bp, synthetic construct
  misc_feature 1..395: note = Synthetic: Beta globin polA signal
  source 1..395: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 39 - DNA, 4209 bp, synthetic construct
  source 1..4209: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgctcgaggccgccaccatggtcagcaagggcgaggaactgttcaccggggtggtgcccatcctggtcgagctggacggcgacgtaaacggccacaagttcagcgtgtccggcgagggcgagggcgatgccacctacggcaagctgaccctgaagttcatctgtaccaccggcaagctgcccgtgccctggcccaccctcgtgaccaccctgacctacggcgtgcaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcaactcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggcggccgccgccggtgtagttgattaactcagctacttaataattaggtgaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 40 - DNA, 7006 bp, synthetic construct
  source 1..7006: mol_type = other DNA
tcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgctcgaggccgccaccatggtcagcaagggcgaggaactgttcaccggggtggtgcccatcctggtcgagctggacggcgacgtaaacggccacaagttcagcgtgtccggcgagggcgagggcgatgccacctacggcaagctgaccctgaagttcatctgtaccaccggcaagctgcccgtgccctggcccaccctcgtgaccaccctgacctacggcgtgcaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcaactcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggcggccgccgccggtgtagttgattaactcagctacttaataattaggtgaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 41 - DNA, 3494 bp, synthetic construct
  source 1..3494: mol_type = other DNA
tcagaagaactcatcgagcatcaagtgaaactgtagcttgttcatatcaggattatcaattccgtatttctgaaatagccgtttctgcagtgatggagagaactcgccgaggcagttccagaggatggcaagatcctggtatcggtcagcgattcccactcgtcctacgtcaatacagcctattaatttcccctcgtcaaaaataaggttatcaagtgagaaatcgccgtgagtcacgacactgtcaggactgaatggcaaaagtttatgcatttccttccagacttgttccacaggccagccattccgctcgtcgtcgaagtcactggcgtcaaccagaccgttattcatcctagattgtgcctgagcgagacgaaatacgcggtcgctgttaaaggggcagttacacacaggaatggaatgcaaccggcgcaggaacactgccagggcatccacaatattttcacctgaatcaggatactcttccagtacctggaatgctgtctttccggggatggcagtggtgagtaaccatgcatcatctggagtacggataaagtgcttgatggtgggcagtggcataaattctgtcagccagttgagtctgaccatctcgtcggtaacgtcattggcaacgctaccctttccatgcttcagaaacaactctggagcatcgggcttgccatacaggcgatagattgtcgcaccggactgccccacattgtccctagcccatttgtacccatacagatcagcatccatgttggaattcagtctcggcctagacgtttctctttgtatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgtaagtttagtctttttgtcttttatttcaggtcccggatccggtggtggtgcaaatcaaagaactgctcctcagtggatgttgcctttacttctagctcgagccaccatggcggggaggagtggggacagtgacgaggacctcctgaaggccgtgaggctgatcaagttcctgtaccagtccaaccctccccctaaccccgaggggacccgtcaggcccgtcggaaccggcggagacggtggagggagagacagagacagatccactccatctccgagcggatcctgtccacctacctgggacggtccgccgaacccgtccccctccagctccctcccctggagagactgaccctggactgcaacgaggactgcgggacctccgggacccagggggtggggtctccccagatcctggtggagtcccccaccatcctggagtccggggccaaggagtgagcggccgcgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaaagcttggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 42 - DNA, 5531 bp, synthetic construct
  source 1..5531: mol_type = other DNA
tcagaaaaactcatcgagcatcagatggaactgcaatttattcatgtcagggttatctatgccgtatttttggaaaagccgcttctgcagtgagggagaaaactcaccgaggcagttccagaggatggcaagatcctggtacctgtcggcgattccgactcgtcccacgtcaatgcagcctatgagtttcccctcgtcgaaaataaggttatccagtgagaaatccccgtgagtcacgaccgaatccggactgaatggcaacagcttgtgcatttccttccacacctgctccacaggccagccatttctctcgtcgtcaaaatcgctggcatcaaccagaccgttgttcattctggattgagcctgagcgagacgaaatacgcggtcgctgttaaatggacagttacacacgggaatagagtgcaatctgcgcaggaacactgccagagcgtcaacaatattttcgccactatcgggatattcttctagtacctggaatgctgtctttccggggatcgcagtggtgagtaaccatgcgtcatcaggggtacggatgaaatgcttgatggtcggcagaggcataaattcggtcagccagtttagtctgaccatctcatctgtcacatcattggcaacgctgccttttccgtgcttcagaaacagctctggggcgtcgggcttcccgtacaacctatagattgtggcgccggactgcccgacattgtcccgagcccatttatacccgtacaggtcagcgtccatgttggaatttaatcgtggcctggacgtctcccgctgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgagccaccatgaaatgtttattgtacttggcctttttgttcatcggggtgaactgcaagttcaccatcgtgttcccccacaaccagaaggggaactggaagaacgtcccctccaactaccactactgcccctcctccagcgacctgaactggcacaacgacctgatcgggaccgccctccaggtgaagatgcccaagagccacaaggccatccaggccgacgggtggatgtgccacgcctccaagtgggtgaccacctgcgacttccggtggtacggccccaagtacatcacccactccatccggtccttcaccccctccgtggagcagtgcaaggagtccatcgagcagaccaagcaggggacctggctgaaccccgggttccctccccagtcctgcgggtacgccaccgtgaccgacgccgaggccgtgatcgtccaggtgacccctcaccacgtcctggtggacgagtacaccggggagtgggtggactcccagttcatcaacgggaagtgctccaactacatctgccccaccgtccacaactccaccacctggcactccgactacaaggtgaagggcctgtgcgactccaacctgatctccatggacatcaccttcttctccgaggacggggagctgtcctccctggggaaggaggggaccgggttccggtccaactacttcgcctacgagaccgggggcaaggcctgcaagatgcagtactgcaagcactggggcgtgaggctcccctccggggtgtggttcgagatggccgacaaggacctgttcgccgccgctcggttccccgagtgccccgaggggtcctccatctccgccccctcccagacctccgtggacgtgtccctgatccaggacgtggagcggatcctggactactccctgtgccaggagacctggtccaagatccgggccggactccccatcagccccgtggacctgtcctacctggcccccaagaaccccgggaccggacccgccttcaccatcatcaacgggaccctgaagtacttcgagacccggtacatccgggtggacatcgccgcccccatcctgtcccggatggtggggatgatctccgggaccaccaccgagagggagctgtgggacgactgggccccctacgaggacgtggagatcggacccaacggggtcctccggacctccagcgggtacaagttccccctgtacatgatcggccacgggatgctggactccgacctccacctgtcctccaaggcccaggtgttcgagcacccccacatccaggacgccgcctcccagctccccgacgacgagtccctgttcttcggggacaccggcctgtccaagaaccccatcgagctggtggaggggtggttctcctcctggaagtcctccatcgcctccttcttcttcatcatcggcctgatcatcggcctgttcctggtcctccgggtggggatccacctgtgcatcaagctgaagcacaccaagaagcggcagatctacaccgacatcgagatgaaccggctggggaagtgagcggccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 43 - DNA, 8502 bp, synthetic construct
  source 1..8502: mol_type = other DNA
tcagaaaaactcgtcgagcatcagatgaaactgcaacttattcatatcggggttatcaattccatatttttggaaaagcctcttctgtaaactaggagaaaactcgccgaggcagttccataggatggccagatcctggtatctgtctgcgattccaacccttcccacatcaatacaacctataagtttgccctcgtcaaaaatcaggttatcaagagagaaatcaccgtgagtgacgactgagtctggactgaatggcaaaagtttgtgcatttctttccagacctgttccacgggccagccatttctctcgtcatcgaaatcactagcatccaccaaaccgttattcattcgtgactgggcctgagcgagacgaaatacgcgatcgctgttaaaagggcaattacaaacaggaatggaatgcaaccgtctcaggaacactgccagagcgtcaacaatattttcgcctgaatcagggtattcttccagtacctggaatgctgtctttccggggatcgcagtggtgagtaaccatgcgtcatctggagtacggataaaatgcttgatggttggaagaggcataaattcggtcagccagtttagtctcaccatctcatctgtcacatcattggcaacgctaccttttccatgcttcaggaacagctctggagcatcgggcttcccatacaatcggtagattgtggctccagattgcccgacattgtcgcgagcccatttatacccatataagtcagcgtccatgttggaattgagtctcggcctggacgtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcttatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcgccgccaccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaaagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtttaaacggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 44 - DNA, 5122 bp, synthetic construct
  source 1..5122: mol_type = other DNA
tccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtnnnnnnnnnngcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctta

SEQ ID NO: 45 - DNA, 1165 bp, synthetic construct; identical to SEQ ID NO: 29
  source 1..1165: mol_type = other DNA

SEQ ID NO: 46 - DNA, 659 bp, synthetic construct; identical to SEQ ID NO: 2
  source 1..659: mol_type = other DNA

SEQ ID NO: 47 - DNA, 758 bp, synthetic construct
  source 1..758: mol_type = other DNA
cggtcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccgcggccgcccaccgtaaggaggaaggaggctgaggggagggggcccctgggagggagcctgccctgggttgctaaccatctcctctctgccaaaag

SEQ ID NO: 48 - DNA, 754 bp, synthetic construct
  source 1..754: mol_type = other DNA
cggtcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccgcggccgcccaccaaggaggaaggaggctgaggggagggggcccctgggagggagcctgccctgggttgctaaccatctcctctctgccaaa

SEQ ID NO: 49 - DNA, 487 bp, synthetic construct
  source 1..487: mol_type = other DNA
ctcgagaagcttgatatcgaattcccacggggttggacgcgtcttaattaaggatccgaacagagagacagcagaatatgggccaaacaggatatctgtggtaagcagttcctgccccgctcagggccaagaacagttggaacagcagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcgc

SEQ ID NO: 50 - DNA, 616 bp, synthetic construct
  source 1..616: mol_type = other DNA
cggtgccaacagatgaggttcacaatctcttccacaaaacatgcagttaaatatctgaggatattcagggacttggatttggtggcaggagatcaacataaaccaagacaaggaagaagtcaaagaaatgaatcaagtagattctctgggatataaggtagggggattggggggttggatagtgcagagtatggtactggcctaaggcactgaggatcatccttttcccacacccaccagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgacg

SEQ ID NO: 51 - DNA, 513 bp, synthetic construct
  source 1..513: mol_type = other DNA
aattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 52 - DNA, 2941 bp, synthetic construct
  source 1..2941: mol_type = other DNA
tgcacgctttccagtctttcagcgtaagccagagtcattcccaaggatgctggtttctctctgggggaagagctgctctgtgatggagcccatgcgtgtcatctgagcctctggcttccctgccagtgcagccctggcagtgtcctacttcccagggctgttgtctgcctggcgggaaggtcctgggcaaaggatcagtctttgtactctgagagcagactacttggctcctctctgttttttatcagcgaagttggatatatctctcccacatttccctaatcatatgctatatattggctttttttttcttctctagcccccaaatacatcaagatgtttgtactggatgaagctgacgaaatgttaagccgtggattcaaggaccagatctatgacatattccaaaagctcaacagcaacacccaggtgagggcagtcttgcttgaatagctaatgattcttgaaaaatagtaagtgccaggggaaccatatactggattcttgagcctttttatgcatctgcttcagttttaggtgtggctagggaagggagcaggcctcaggaaggaaccagcactctaagactggcctttttttccactaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggtggggcccagtgcaggaggcgggcctggtagtgagttgttgggtatagcccctgactgatttttgtcccccaacctccaggagtggaagctggacacactatgtgacttgtatgaaaccctgaccatcacccaggcagtcatcttcatcaacacccggaggaaggtggactggctcaccgagaagatgcatgctcgagatttcactgtatccgccatggtgtgtttgcccgctgccagcctgttgtgggtctgcccgtcagaagtgtcctacttgaagccagggttcctggaacccaggtgcctacctggtctgctgcatatttgttttctcttccagcatggagatatggaccaaaaggaacgagacgtgattatgagggagtttcgttctggctctagcagagttttgattaccactgacctgctggtgagtagagggaactgatagcaaaggcagaagggaggatccaaggtgattccctctccaaggggacatcagtgcctctcaggaaagtagcagcttggaatagaatctggcatgcctaaggcctttggggaactgggatgcttatttcctctgccttccttggctgcccacatggatgcctaagtgtcttccctccgggatagagtgtcctccgtgcacatgctgaagagttgtctttcttgacgtaggccagaggcattgatgtgcagcaggtttctttagtcatcaactatgaccttcccaccaacagggaaaactatatccacaggtaagcgtagatctggaacactcccctaccccttcacacctggccctccctgggcttaaagctcctgatattcctcatccccttccttgttttccagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctgaggggctgtcctgccacccagccccagccagggctcaatctctgggggctgaggagcagcaggaggggggagggaagggagccaagggatggacatcttgtcattttttttctttgaataaatgtcactttttgaggcaaaagaaggaaccgtgaacattttagacacccttttctttggggtaggctcttgccccaggcgccggctcttctcccaaaaaaaaaaaaaaaacactaatccatttccctaacctagtaacctccagatcccagaggctctcctcacctcagctgagctcctttgaaagtgattcaagggactatgtcactcagcctcatttgctggaccaaatctggagggagaacccctaaaacccctaagtgaggttgcccagggggttgtccccaggtggggggaagcaggggagagaaaatggtagccatttttacattgttttgtatagtatttattgattcaggaaacaaacacaaaattctgaataaaatgacttggaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcc

SEQ ID NO: 53 - DNA, 4404 bp, synthetic construct
  source 1..4404: mol_type = other DNA
atgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 54 - DNA, 4404 bp, synthetic construct; identical to SEQ ID NO: 3
  source 1..4404: mol_type = other DNA

SEQ ID NO: 55 - DNA, 4404 bp, synthetic construct; identical to SEQ ID NO: 30
  source 1..4404: mol_type = other DNA

SEQ ID NO: 56 - DNA, 4410 bp, synthetic construct
  source 1..4410: mol_type = other DNA
atgcaactggaactgtcaacatgtgtcttcttgtgcctgctgcccctgggcttctctgccatcaggaggtactacctgggggctgtggagctgtcctgggactacaggcagtctgagctgctgagggagctgcatgtggacaccaggttccctgccacagcccctggggccctgcccctgggcccctctgtgctgtacaagaagacagtgtttgtggagttcacagaccagctgttctctgtggccaggcccaggcccccctggatgggcctgctgggccccaccatccaggctgaggtgtatgacacagtggtggtgaccctgaagaacatggcctcccaccctgtgtccctgcatgctgtgggggtgtccttctggaagtcctctgagggggctgagtatgaggaccacaccagccagagggagaaggaggatgacaaggtgctgcctggcaagagccagacctatgtgtggcaggtgctgaaggagaatggccccacagcctctgaccccccctgcctgacctacagctacctgtcccatgtggacctggtgaaggacctgaactctggcctgattggggccctgctggtgtgcagggaggggtccctgaccagggagaggacccagaacctgcatgagtttgtgctgctgtttgctgtgtttgatgagggcaagagctggcactctgccaggaatgactcctggaccagggccatggaccctgcccctgccagggcccagcctgccatgcacacagtgaatgggtatgtgaacaggagcctgcctggcctgattgggtgccacaagaagtctgtgtactggcatgtgattgggatgggcacctcccctgaggtgcactccatcttcctggagggccacaccttcctggtgaggcaccacaggcaggcctccctggagatcagccccctgaccttcctgacagcccagaccttcctgatggacctgggccagttcctgctgttctgccacatcagcagccaccaccatgggggcatggaggcccatgtgagggtggagtcctgtgctgaggagccccagctgaggaggaaggctgatgaggaggaggactatgatgacaacctgtatgactctgacatggatgtggtgaggctggatggggatgatgtgtcccccttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtgaggttcatggcctacacagatgagaccttcaagaccagggaggccatccagcatgagtctggcatcctgggccccctgctgtatggggaggtgggggacaccctgctgatcatcttcaagaaccaggcctccaggccctacaacatctacccccatggcatcacagatgtgaggcccctgtacagcaggaggctgcccaagggggtgaagcacctgaaggacttccccatcctgcctggggagatcttcaagtacaagtggacagtgacagtggaggatggccccaccaagtctgaccccaggtgcctgaccaggtactacagctcctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgacagagaacatccagaggttcctgcccaaccctgctggggtgcagctggaggaccctgagttccaggcctccaacatcatgcactccatcaatgggtatgtgtttgactccctgcagctgtctgtgtgcctgcatgaggtggcctactggtacatcctgtccattggggcccagacagacttcctgtctgtgttcttctctggctacaccttcaagcacaagatggtgtatgaggacaccctgaccctgttccccttctctggggagacagtgttcatgagcatggagaaccctggcctgtggatcctggggtgccacaactctgacttcaggaacaggggcatgacagccctgctgaaggtgtcctcctgtgacaagaacacaggggactactatgaggactcctatgaggacatctctgcctacctgctgtccaagaacaatgccattgagcccaggtcctttgcccagaacagcaggcccccctctgcctctgcccccaagccccctgtgctgaggaggcaccagagggacatcagcctgcccaccttccagcctgaggaggacaagatggactatgatgacatcttctccacagagaccaagggggaggactttgacatctatggggaggatgagaaccaggaccccaggagcttccagaagaggaccaggcactacttcattgctgctgtggagcagctgtgggactatggcatgtctgagtcccccagggccctgaggaacagggcccagaatggggaggtgcccaggttcaagaaggtggtgttcagggagtttgctgatggctccttcacccagccctcctacaggggggagctgaacaagcacctgggcctgctgggcccctacatcagggctgaggtggaggacaacatcatggtgaccttcaagaaccaggcctccaggccctacagcttctacagcagcctgatcagctaccctgatgaccaggagcagggggctgagcccaggcacaactttgtgcagcccaatgagaccaggacctacttctggaaggtgcagcaccacatggcccccacagaggatgagtttgactgcaaggcctgggcctacttctctgatgtggacctggagaaggatgtgcactctggcctgattggccccctgctgatctgcagggccaacaccctgaatgctgcccatgggaggcaggtgacagtgcaggagtttgccctgttcttcaccatctttgatgagaccaagagctggtacttcacagagaatgtggagaggaactgcagggccccctgccacctgcagatggaggaccccaccctgaaggagaactacaggttccatgccatcaatgggtatgtgatggacaccctgcctggcctggtgatggcccagaaccagaggatcaggtggtacctgctgtccatgggctccaatgagaacatccactccatccacttctctggccatgtgttctctgtgaggaagaaggaggagtacaagatggctgtgtacaacctgtaccctggggtgtttgagacagtggagatgctgccctccaaggtgggcatctggaggattgagtgcctgattggggagcacctgcaggctggcatgagcaccaccttcctggtgtacagcaagaagtgccagacccccctggggatggcctctggccacatcagggacttccagatcacagcctctggccagtatggccagtgggcccccaagctggccaggctgcactactctggctccatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaactccacaggcaccctgatggtgttctttgggaatgtggactcctctggcatcaagcacaacatcttcaacccccccatcattgccaggtacatcaggctgcaccccacccactacagcatcaggagcaccctgaggatggagctgatggggtgtgacctgaacagctgcagcatgcccctggggatggagtccaaggccatctctgatgcccagatcacagcctccagctacttcaccaatatgtttgccacctggagcccctccaaggccaggctgcacctgcagggcaggtccaatgcctggaggccccaggtgaacaaccccaaggagtggctgcaggtggacttccagaagaccatgaaggtgacaggggtgaccacccagggggtgaagagcctgctgaccagcatgtatgtgaaggagttcctgatcagcagcagccaggatggccaccagtggaccctgttcttccagaatgggaaggtgaaggtgttccaggggaaccaggactccttcacccctgtggtgaacagcctggacccccccctgctgaccaggtacctgaggatccacccccagtcctgggtgcaccagattgccctgaggatggaggtgctggggtgtgaggcccaggacctgtactgataatag

SEQ ID NO: 57 - DNA, 32 bp, synthetic construct
  source 1..32: mol_type = other DNA
actcttctggtccccacagactcagagagaac

SEQ ID NO: 58 - DNA, 134 bp, synthetic construct
  source 1..134: mol_type = other DNA
gctcgctttcttgctgtccaatttctattaaaggttcctttgttccctaagtccaactactaaactgggggatattatcaagggccttgagcatctggattctgcctaataaaaaacatttattttcattgcaa

SEQ ID NO: 59 - DNA, 268 bp, synthetic construct
  source 1..268: mol_type = other DNA
gctcgctttcttgctgtccaatttctattaaaggttcctttgttccctaagtccaactactaaactgggggatattatcaagggccttgagcatctggattctgcctaataaaaaacatttattttcattgcaagctcgctttcttgctgtccaatttctattaaaggttcctttgttccctaagtccaactactaaactgggggatattatcaagggccttgagcatctggattctgcctaataaaaaacatttattttcattgcaa

SEQ ID NO: 60 - DNA, 591 bp, synthetic construct
  source 1..591: mol_type = other DNA
atcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 61 - DNA, 4209 bp, synthetic construct; identical to SEQ ID NO: 39
  source 1..4209: mol_type = other DNA

SEQ ID NO: 62 - DNA, 9919 bp, synthetic construct
  source 1..9919: mol_type = other DNA
cggtcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccgcggccgcaaaacctctcgagccaccatgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtcttacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgcccagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccccctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttacgaagatatttcagcatacttgctgagtaaaaacaatgccattgaacctaggagctttgcccagaattcaagaccccctagtgcgagcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccatctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaagaagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagcgccggtgaagggcgaattccagcacactggcggccgttactagtggatccgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaatctgcagaattcgcccttatcgat

SEQ ID NO: 63 - DNA, 5127 bp, synthetic construct; identical to SEQ ID NO: 22
  source 1..5127: mol_type = other DNA

SEQ ID NO: 64 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

(19) United States
(12) Patent Application Publication
(10) Pub. No.: US 2026/0275376 A1
(43) Pub. Date: Sep. 17, 2026

(54) Lentiviral System

(71) Applicant: Expression Therapeutics, LLC, Tucker, GA (US)

(72) Inventors: Harrison BROWN, Atlanta, GA (US); Gabriela DENNING, Tucker, GA (US)

(21) Appl. No.: 19/162,042

(22) PCT Filed: Apr. 9, 2024
(86) PCT No.: PCT/US2024/023762

§ 371 Date: Sep. 4, 2025

Related U.S. Application Data
(60) Provisional application No. 63/444,745, filed on Feb. 10, 2023.
(60) Provisional application No. 63/617,361, filed on Jan. 3, 2024.

(51) Int. Cl. C12N 15/86; A61K 38/37; A61K 48/00; C07K 14/005; C07K 14/755

(52) U.S. Cl. CPC C12N 15/86; A61K 38/37; A61K 48/0033; A61K 48/005; C07K 14/005; C07K 14/755; C12N 2740/15043; C12N 2740/16022; C12N 2760/20222

(57) ABSTRACT

We disclose a lentiviral vector, for use in research, clinical, industrial, and other suitable applications. The novel lentiviral vectors disclosed herein introduce numerous novel elements which increase the safety profile of the vector without reducing the efficacy of the system. The novel lentiviral vectors are useful as safe and highly efficient transduction vectors for any application using or benefitting from transduction. We also disclose methods and compositions are provided that allow for high-level expression of a factor VIII polypeptide.

### RELATED APPLICATIONS

The present patent document claims the benefit of the filing date under 35 U.S.C. § 119 (e) of Provisional U.S. Patent Application Ser. No. 63/444,745, filed Feb. 10, 2023, Provisional U.S. Patent Application Ser. No. 63/617,361, filed Jan. 3, 2024 each of which is hereby incorporated by reference in its entirety.

### REFERENCE TO APPENDIX SEQUENCE LISTING

The Sequence Listing associated with this application is provided is hereby incorporated by reference into the specification. The name of the file containing the Sequence Listing is SEQ_103_LentPCT3 225 KB. It is being submitted electronically via Patent Center. The information in electronic format of the Sequence Listing and in the specification is incorporated herein by reference in its entirety.

### BACKGROUND

Viral vectors are a tool for delivering genetic material to target cell populations. Currently, retroviral vectors comprise approximately half of the viral gene therapy vectors in development pipelines. Of the viral vectors under development, about half are retroviral or lentiviral vectors. Retroviral vectors currently account for half of the approved gene therapy products to date.

Factor VIII is a large (~ 300 kDa) glycoprotein that functions as an integral component of the intrinsic pathway of blood coagulation. It contains a series of domains designated A1-A2-B-ap-A3-C1-C2. The B domain of factor VIII has no known function and can be deleted without loss of coagulant activity. Mutations in the factor VIII gene that result in decreased or defective factor VIII protein give rise to the genetic disease, hemophilia A, which is characterized by recurrent bleeding episodes. Treatment of hemophilia A requires intravenous infusion of either plasma-derived or recombinant factor VIII.

Since the introduction of recombinant factor VIII for the treatment of hemophilia A, supply has struggled to keep up with demand because factor VIII is expressed and recovered at low levels in the heterologous mammalian cell culture systems used for commercial manufacture (Garber et al. (2000) Nature Biotechnology 18:1133). Additionally, factor VIII levels during hemophilia A gene therapy trials indicate that expression levels will be a limiting feature (Roth, et al. (2001) N. Engl. J. Med. 344:1735-1742). The importance of this problem has resulted in significant research efforts to overcome the low factor VIII expression barrier. Several factors that limit expression have been identified, including low mRNA levels (Lynch et al. (1993) Hum. Gene Ther. 4:259-272; Hoeben et al. (1995) Blood 85:2447-2454; Koeberl et al. (1995) Hum. Gene Ther. 6:469-479), interaction with protein chaperones and inefficient secretion (Pipe et al. (1998) J. Biol. Chem. 273:8537-8544; Tagliavacca et al. (2000) Biochemistry 39:1973-1981; Kaufinan et al. (1997) Blood Coagul Fibrinolysis 8 Suppl 2: S3-14) and rapid decay in the absence of von Willebrand factor (Kaufman et al. (1988) J. Biol. Chem. 263:6352-6362 and Kaufman et al. (1989) Mol. Cell Biol. 9:1233-1242). Deletion of the B-domain has been shown to increase factor VIII protein production in heterologous systems (Toole et al. (1986) Proc. Natl. Acad. Sci. U.S.A. 83:5939-5942). A B-domain deleted form of human factor VIII (Lind et al. (1995) Eur. J. Biochem. 232:19-27) has been approved for clinical use.

Despite these insights into factor VIII regulation, expression continues to be significantly lower than other recombinant proteins in the heterologous systems used in commercial manufacture (Kaufman et al. (1997) Blood Coagul. Fibrinolysis 8 Suppl 2: S3-14), as well as in ex-vivo (Roth, et al. (2001) N. Engl. J. Med. 344:1735-1742) and in vivo gene therapy applications (Chuah et al. (1995) Hum. Gene Ther. 6:1363-1377). Methods and compositions are needed for the increased expression of factor VIII.

### BRIEF SUMMARY

We disclose a viral vector, particularly a lentiviral vector, for use in research, clinical, industrial, and other suitable applications. The novel lentiviral vectors disclosed herein are useful as highly efficient transduction vectors for any application using or benefitting from transduction. Non limiting examples of potential uses of the disclosed viral vector include gene therapy, production of recombinant proteins, cancer treatment, and other manufacturing, experimental, preventative, elective, or therapeutic purposes. The lentiviral vectors are disclosed in various examples, including the example of a vector expressing a factor VIII polypeptide. However, the vector can be used outside of factor VIII and hemophilia A therapies.

Also disclosed are methods and compositions are provided that allow for high-level expression of a factor VIII polypeptide. More specifically, we disclose methods and compositions comprising nucleic acid and amino acid sequences comprising a modified factor VIII that results in high-level expression of the polypeptide. The methods and compositions find use in the treatment of factor VIII deficiency, including, for example, hemophilia A.

### BRIEF DESCRIPTION OF THE DRAWINGS

FIG. 1 shows an exemplary HIV-1 virion.

FIG. 2 shows an exemplary retrovirus life cycle.

FIG. 3 shows basic elements of an exemplary lentiviral vector system.

FIG. 4 is a schematic of an exemplary lentiviral vector system.

FIG. 5 is a schematic of an exemplary SIN vector design.

FIG. 6 is a map of an exemplary transfer plasmid.

FIG. 7 is a map of an exemplary GAG/POL plasmid.

FIG. 8 is a map of an exemplary env (VSVG) plasmid.

FIG. 9 is a map of an exemplary transfer plasmid with a fVIII transgene.

FIG. 10 is a map of an exemplary transfer plasmid with a fVIII transgene.

FIG. 11 is a map of an exemplary REV plasmid.

FIG. 12 illustrates a potential protein product avoided by the disruption in RRE.

FIG. 13 illustrates an exemplary transgene insertion site flanked by a six frame triple stop codon (SEQ ID NO: 25).

FIG. 14 illustrates P31 integrate with terminal stop codon (SEQ ID NO: 27).

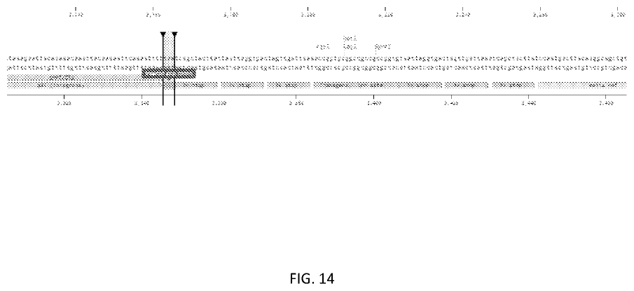

FIG. 15 illustrates an exemplary transgene design.

FIG. 16 illustrates an exemplary GAG/POL design.

FIG. 17 illustrates titers of infection titers using a disclosed lentivirus vector system.

FIG. 18 illustrates, via GFP expression, a comparison of a disclosed lentiviral vector system compared to expression in a commercially available system.

FIG. 19 illustrates, via qPCR titer, a comparison of a disclosed lentiviral vector system compared to expression in a commercially available system.

FIG. 20 illustrates, via Flow titer, a comparison of a disclosed lentiviral vector system compared to expression in a commercially available system.

FIG. 21 illustrates, via Flow titer, a comparison of earlier trial lentiviral systems where CMV promoter was used to drive expression versus two commercially available systems.

FIG. 22 illustrates, via qPCR, a comparison of earlier trial lentiviral systems where CMV promoter was used to drive expression versus two commercially available systems.

FIG. 23 illustrates, via Flow titer, a comparison of earlier trial lentiviral systems where various known promoter combinations were used to drive expression versus two commercially available systems.

FIG. 24 is a chart which demonstrates exemplary arrangements of the elements of various constructs.

FIG. 25 provides a variation of the LentET backbone.

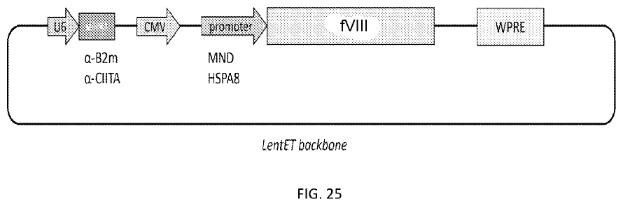

FIG. 26 provides data illustrated a comparison of factor VIII activity between various codon optimized contructs versus non-codon optimized constructs.

FIG. 27 provides a flow chart of the method and system for codon optimization.

FIG. 28 provides data illustrating the difference in vector titer resulting from two vector systems disclosed herein.

FIG. 29 provides vector copy number data for two vector systems disclosed herein.

FIG. 30 is a plasmid map of a transfer vector of a third generation transfer vector system with the GFPBright transgene and WPREmut.

FIG. 31 is a plasmid map of a Gagpol vector member of a third generation vector system.

FIG. 32 is a plasmid map of a VSVG vector member of a third generation vector system.

FIG. 33 is a plasmid map of a UbC rev vector member of a third generation vector system.

FIG. 34 is a graphical representation of a projected structure of an integrated genome from the third generation vector system.

FIG. 35 is a graphical representation of the transfer plasmid of FIG. 6 indicating the transgene insertion site.

### BRIEF DESCRIPTION OF THE SEQUENCES

SEQ ID NO: 1 shows the complete nucleotide sequence of a transfer plasmid of the disclosed lentiviral vector capable of expressing a proprietary liver codon optimized fVIII.

SEQ ID NO: 2 shows a nucleotide sequence of a CD68 promoter.

SEQ ID NO: 3 shows a nucleotide sequence of a liver codon optimized fVIII.

SEQ ID NO: 4 shows the amino acid sequence of a liver codon optimized fVIII.

SEQ ID NO: 5 shows the complete nucleotide sequence of a GAG/POL plasmid.

SEQ ID NO: 6 shows the nucleotide sequence of a six frame stop element.

SEQ ID NO: 7 shows the nucleotide sequence of a hCD8 codon optimized Kanamycin resistance gene.

SEQ ID NO: 8 shows the amino acid sequence of a hCD8 codon optimized Kanamycin resistance peptide.

SEQ ID NO: 9 shows a nucleotide sequence of a CMV promoter and Beta globin intron.

SEQ ID NO: 10 shows a nucleotide sequence of a PGK polyA signal.

SEQ ID NO: 11 shows a nucleotide sequence of GAG.

SEQ ID NO: 12 shows an amino acid sequence of GAG.

SEQ ID NO: 13 shows a nucleotide sequence of POL.

SEQ ID NO: 14 shows an amino acid sequence of POL.

SEQ ID NO: 15 shows a nucleotide sequence of a complete ENV VSVG plasmid.

SEQ ID NO: 16 shows a nucleotide sequence of a human NK cell codon optimized Kanamycin resistance gene.

SEQ ID NO: 17 shows an amino acid sequence of a human NK cell codon optimized Kanamycin resistance peptide.

SEQ ID NO: 18 shows a nucleotide sequence of a PGK promoter and PGK intron.

SEQ ID NO: 19 shows a nucleotide sequence for a 293T codon optimized VSVG.

SEQ ID NO: 20 shows an amino acid sequence for a VSVG.

SEQ ID NO: 21 shows a nucleotide sequence for a human growth hormone poly A.

SEQ ID NO: 22 shows a nucleotide sequence for a complete transfer plasmid with no transgene.

SEQ ID NO: 23 shows a nucleotide sequence for a liver codon optimized Kanamycin resistance gene.

SEQ ID NO: 24 shows a nucleotide sequence for a disrupted HIV antisense protein start codon.

SEQ ID NO: 25 shows a nucleotide sequence for a six frame triple stop codon insulator sequence.

SEQ ID NO: 26 shows a nucleotide sequence for a transgene insertion site.

SEQ ID NO: 27 shows a nucleotide sequence for a P31 integrase with an added stop codon.

SEQ ID NO: 28 shows a complete nucleotide sequence of a transfer plasmid carrying the myeloid codon optimized (MCO) fVIII transgene.

SEQ ID NO: 29 shows the nucleotide sequence of a modified EFla promoter.

SEQ ID NO: 30 shows the nucleotide sequence of a myeloid codon optimized (MCO) fVIII transgene.

SEQ ID NO: 31 shows the amino acid sequence of a MCO fVIII.

SEQ ID NO: 32 shows the nucleotide sequence of a plasmid carrying REV.

SEQ ID NO: 33 shows a nucleotide sequence of a kanamycin resistance gene.

SEQ ID NO: 34 shows an amino acid sequence of a kanamycin resistance gene.

SEQ ID NO: 35 shows a nucleotide sequence of a UbC promoter with a SV40 intron.

SEQ ID NO: 36 shows a nucleotide sequence for a 293T cell codon optimized REV gene.

SEQ ID NO: 37 shows an amino acid sequence for a 293T codon optimized REV peptide.

SEQ ID NO: 38 shows a nucleotide sequence of a Beta globin poly A signal.

SEQ ID NO: 39 shows a nucleotide sequence of a LentET product integrated into a genome.

SEQ ID NO: 40 shows a nucleotide sequence of LentET MND GFPbright WPREmut.

SEQ ID NO: 41 shows a nucleotide sequence for LentET UbC rev ECO-L [PL549].

SEQ ID NO: 42 shows a nucleotide sequence for LentET VSVG ECO-L.

SEQ ID NO: 43 shows a Nucleotide sequence for LentET CMV Gagpol 8.0.

SEQ ID NO: 44 shows a nucleotide sequence of a Lentiviral fVIII transfer cassette backbone.

SEQ ID NO: 45 shows a nucleotide sequence of an Ef1a promoter.

SEQ ID NO: 46 shows a nucleotide sequence of a CD68 promoter.

SEQ ID NO: 47 shows a nucleotide sequence of a CD68 intron promoter.

SEQ ID NO: 48 shows a nucleotide sequence of a CD68 intron no splice promoter.

SEQ ID NO: 49 shows a nucleotide sequence of a MND promoter.

SEQ ID NO: 50 shows a nucleotide sequence of a CD14 promoter.

SEQ ID NO: 51 shows a nucleotide sequence of a mPGK promoter.

SEQ ID NO: 52 shows a nucleotide sequence of a CD68-2.9 promoter.

SEQ ID NO: 53 shows a nucleotide sequence of a fVIII variation designated NoCO.

SEQ ID NO: 54 shows a nucleotide sequence of a fVIII variation designated LCO.

SEQ ID NO: 55 shows a nucleotide sequence of a fVIII variation designated MCO.

SEQ ID NO: 56 shows a nucleotide sequence of a fVIII variation designated 30×.

SEQ ID NO: 57 shows a nucleotide sequence of a 5′ HBB.

SEQ ID NO: 58 shows a nucleotide sequence of a 3′ HBB.

SEQ ID NO: 59 shows a nucleotide sequence of a 2×3′ HBB.

SEQ ID NO: 60 shows a nucleotide sequences of a WPREmut.

SEQ ID NO: 61 shows a nucleotide sequence of a projected integration into the genome.

SEQ ID NO: 62 shows a plasmid with CD68 and an exemplary fVIII.

SEQ ID NO: 63 shows a LentET transfer plasmid with no transgene.

SEQ ID NO: 64 shows BGH polyA.

### DETAILED DESCRIPTION

The use of viral vectors as a means for modification of cells, including but not limited to eukaryotic cells, is common in academia and industry for research, clinical, and manufacturing applications. Lentiviral vectors, derived from the human immunodeficiency virus, are retroviruses.

### Terms

Unless otherwise noted, technical terms are used according to conventional usage. Definitions of common terms in molecular biology may be found in Krebs et al. (eds.), Lewin's genes XII, published by Jones & Bartlett Learning, 2017; Kendrew et al. (eds.), The Encyclopedia of Molecular Biology, published by Blackwell Science Ltd., 2009 (ISBN 9780632021826). The singular terms “a,” “an,” and “the” include plural referents unless context clearly indicates otherwise. “Comprising A or B” means including A, or B, or A and B. Although methods and materials similar or equivalent to those described herein can be used in the practice or testing of the present disclosure, suitable methods and materials are described below. In addition, the materials, methods, and examples are illustrative only and not intended to be limiting. In case of conflict, the present specification, including explanations of terms, will control. In order to facilitate review of the various embodiments of the disclosure, the following explanations of specific terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Administration/Administer: To provide or give a subject an agent, such as a therapeutic agent (e.g. a recombinant viral vector), by any effective route. Exemplary routes of administration include, but are not limited to, injection (such as subcutaneous, intramuscular, intradermal, intraperitoneal, and intravenous), cell transfer, oral, intraductal, sublingual, rectal, transdermal, intranasal, vaginal and inhalation routes.

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Codon-optimized: A “codon-optimized” nucleic acid refers to a nucleic acid sequence that has been altered such that the codons are optimal for expression in a particular system (such as a particular species or group of species or a particular tissue type or cell type or group of cells). For example, a nucleic acid sequence can be optimized for expression in mammalian cells or in a particular mammalian species (such as human cells). Alternatively, or additionally, a nucleic acid can be optimized for expression in a particular organ, organ system, environment, tissue or cell type. Codon optimization does not alter the amino acid sequence of the encoded protein.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a nucleic acid sequence (including but not limited to a heterologous nucleic acid sequence) to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, RNA and mRNA) or a defined sequence of amino acids and the biological properties resulting therefrom.

Operably or Operatively linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. The term “operatively” linked is used interchangeably herein.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed vectors.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as vector compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Purified: The term purified does not require absolute purity; rather, it is intended as a relative term. Thus, for example, a purified protein preparation is one in which the protein is more enriched than the peptide or protein is in its natural environment within a cell. In one embodiment, a preparation is purified such that the protein represents at least 50% of the total protein content of the preparation.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g. a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell.

Protein: A biological molecule expressed by a gene or other encoding nucleic acid (e.g., a cDNA) and comprised of amino acids.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals.

Therapeutically effective amount: The amount of agent that is sufficient to prevent, treat (including prophylaxis), reduce and/or ameliorate the symptoms and/or underlying causes of a disorder or disease.

It is understood that to obtain a therapeutic response to the disease or condition can require multiple administrations of a therapeutic agent. Thus, a therapeutically effective amount encompasses a fractional dose that contributes in combination with previous or subsequent administrations to attaining a therapeutic outcome in the patient. For example, a therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is a lentiviral vector. In some embodiments, the vector is a gamma-retroviral vector, a lentiviral vector, or an adenoviral vector.

### Lentiviral Vector

Retroviruses initiate as RNA viruses that convert their RNA genome into a DNA intermediate via reverse transcriptase. The resultant DNA can then be stably integrated into the genome of the host cells in a semi-random pattern. The host cell then considers the integrated viral genome as part of its own. Therefore, the genetic regulatory elements contained within the proviral genome may affect the expression of nearby genes.

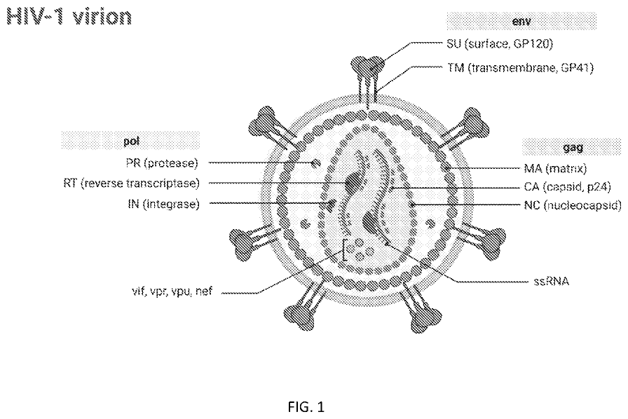

Turning to FIG. 1, using HIV-1 as a non-limiting example, we demonstrate the basic elements of the exemplary retrovirus. An outer lipid envelope may contain transmembrane and surface proteins encoded by the envelope (env) gene. The env gene may direct tropism. For example, the env gene may direct the virus to infect a particular cell, tissue, or host species. An inner proteinaceous core may include at least one of matrix, nucleocapsid and capsid proteins encoded by the gag gene, and protease, integrase and reverse transcriptase proteins encoded by the pol gene.

Another genetic element found in retroviruses is the long terminal repeat (LTR) that is present on each end and possesses both promoter and enhancer activities. To summarize, basic genes useful for retroviral and lentiviral survival and function are the gag, pol, and env genes. The gag gene encodes structural proteins, pol encodes enzymes required for reverse transcriptase and integration into the host cell genome. The env gene encodes the viral envelope glycoprotein.

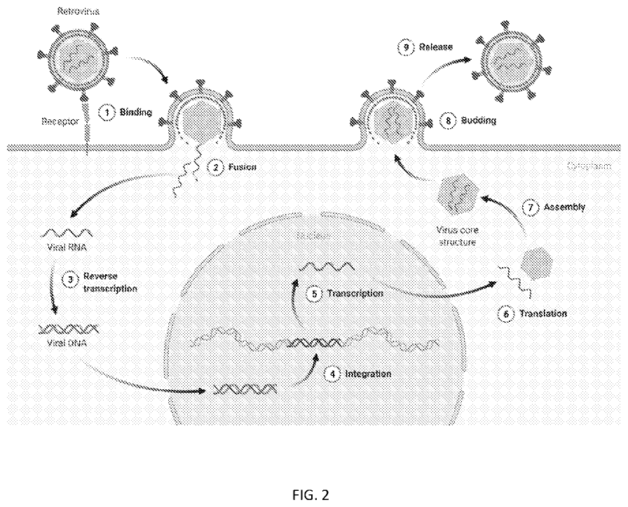

Turning to FIG. 2, the life cycle of the retrovirus includes entry by the mature virus into a cell either through membrane fusion or receptor-mediated endocytosis. In an exemplary system, after fusion, the virus proteins dissociate from the viral core. Reverse transcriptase facilitates conversion of the viral RNA into double stranded DNA. Proviral DNA complexes with the viral proteins and are transported into the host cell nucleus where it may be integrated into the host genome. Accessory viral proteins including integrase together with endogenous host cell factors assist integration of the proviral DNA into the host genome. The integrated proviral genome of the, e.g., unmodified retrovirus system, relies on host machinery for transcription and translation of viral proteins necessary to assemble infectious particles or virions. The resulting virions are released into the extracellular space from the plasma membrane through a process called budding. During the budding process host cell proteins may be incorporated into the virion envelope.

Reverse transcription and integration are useful for lentiviral vector function. Following uncoating, the remaining viral nucleic acid and protein complex is often referred to as the reverse transcription complex (RTC). This RTC is actively transported to the host cell chromosomal DNA, where integration may occur. Turning to FIG. 2, steps 3 through 6, The process of reverse transcription of viral RNA to double-stranded viral DNA relies on multiple priming steps. A transfer RNA binds to the primer-binding site at the 5′ end of the viral RNA genome. The reverse transcriptase synthesizes a negative-strand of viral DNA (FIG. 2, 3). The viral RNA is degraded. The resulting single strand DNA is subsequently transferred to the 3′ end of the viral RNA to serve as a primer for the synthesis of the negative-strand viral DNA, which restores the U3RU5 sequence of the long terminal repeat (LTR). RNase H-resistant polypurine tracts primes the synthesis of the positive-strand viral DNA. Integration of the viral DNA involves the steps of tethering, 3′ processing/cleavage of a precise number of terminal nucleotides, strand transfer, and DNA repair.

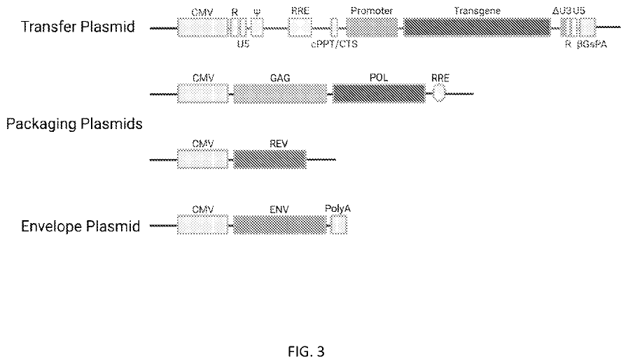

FIG. 3 illustrates the basic elements of a replication-deficient lentiviral vector system. The viral genome may be divided into separate plasmids to reduce incidence of generating recombinant virus. In this example, the viral genome is divided into three (3) separate plasmids and delivered with a separate therapeutic transgene transfer plasmid. It will be understood by one of skill in the art that the genetic elements may be further divided, combined, or reorganized onto more or fewer plasmids. In this exemplary system, the vector transfer plasmid encoding the gene of interest is operatively linked to a lentiviral LTR sequences. In this example, the vector further separates the genes encoding GAG and POL onto one plasmid separate from the individual plasmids encoding each of REV and ENV. In this example, the env gene is derived from the vesicular stomatitis virus and is referred to herein as VSVG or env (VSVG).

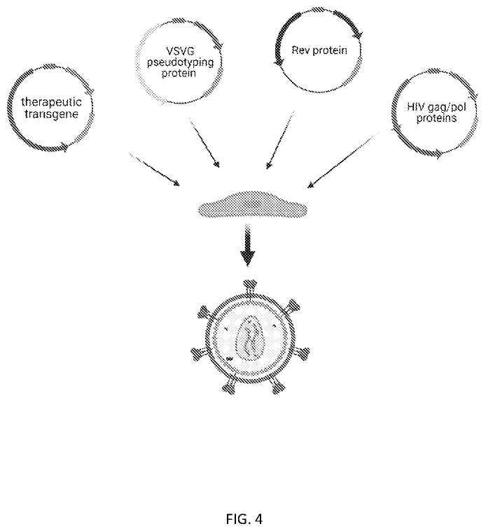

FIG. 4 is a further generic representation of the disclosed system. Theoretical safety issues that are addressed and ameliorated by the disclosed lentiviral system include but are not limited to the presence or development of replication competent virus, insertional mutagenesis, and confirmation of vector identity, purity, and manufacturing consistency. The therapeutic transgene transfer plasmid packages the desired gene that is ultimately integrated and expressed in the host cell DNA. Thus, of the various plasmids in the lentiviral vector system, the therapeutic transgene transfer plasmid has the highest safety concerns. We disclose herein various novel modifications to the lentiviral vector system to address these safety concerns.

Turning to FIG. 4, the disclosed lentiviral vector system includes a therapeutic protein transfer plasmid capable of carrying the transgene of interest; a plasmid encoding ENV, referred to herein as the VSVG pseudotyping protein; a plasmid encoding the REV protein; and a plasmid encoding the GAG and POL proteins. Other plasmids may be included and still fall within the disclosed system and/or the disclosed recombinantly modified genes may be broken into more or combined into fewer plasmids or reorganized. One significantly unique factor of the disclosed system of FIG. 4 is that it is a four plasmid system, where the four plasmids are the therapeutic protein transfer plasmid capable of carrying the transgene of interest; a plasmid encoding ENV, referred to herein as the VSVG pseudotyping protein; a plasmid encoding the REV protein; and a plasmid encoding the GAG and POL proteins.

Conventional lentiviral systems are nearly identical in design. To avoid copying conventional systems, the original wild type HIV genome was adopted as a starting point for modification. This removed the various single nucleotide polymorphisms (SNPs) that are ubiquitous in conventional lentiviral systems. Rather than adapting pre-existing lentiviral materials, the disclosed lentiviral system is de novo. They were custom synthesized from wild type sequence as compared to making additive, subtractive, or rearrangement changes to an existing system.

Starting with the HIV-1 HBX2 genome, we designed the disclosed novel retroviral vector. We first designed a transfer cassette which supplied the minimal cis-regulatory elements useful to support viral particle formation and proviral genome integration while eliminating or replacing genes related to viral replication. This was accomplished by removing most of the viral protein encoding genetic sequence from the vector genome. A further step to eliminate viral replication was placing the required elements into separate plasmids or integrated cistrons. Here the vector components were split between 4 or more distinct elements and in some cases each element has been further modified at the nucleic acid level to reduce homology between the individual components or with natural viral sequences that could be present. (See, e.g, FIG. 4). Reference to four or more distinct elements refers to the fact that there are four distinct plasmids in the system. In this variation they are the therapeutic protein transfer plasmid capable of carrying the transgene of interest; a plasmid encoding ENV, referred to herein as the VSVG pseudotyping protein; a plasmid encoding the REV protein; and a plasmid encoding the GAG and POL proteins. Each of these four plasmids has been optimized at the nucleic acid level to reduce homology between, for example, the therapeutic protein transfer plasmid capable of carrying the transgene of interest versus the plasmid encoding ENV, referred to herein as the VSVG pseudotyping protein; and the plasmid encoding the REV protein; and the plasmid encoding the GAG and POL proteins. Similarly, the plasmid encoding ENV, referred to herein as the VSVG pseudotyping protein, has been optimized at the nucleic acid level to reduce homology between, for example, it and the therapeutic protein transfer plasmid capable of carrying the transgene of interest versus; the plasmid encoding the REV protein; and the plasmid encoding the GAG and POL proteins. Similarly, the plasmid encoding the REV protein has been optimized at the nucleic acid level to reduce homology between, for example, it and the plasmid encoding ENV, referred to herein as the VSVG pseudotyping protein; the therapeutic protein transfer plasmid capable of carrying the transgene of interest versus; and the plasmid encoding the GAG and POL protein. And so on, each plasmid has been codon optimized to reduce homology between it and each other plasmid in the system.

FIG. 4 depicts a four plasmid, lentiviral vector production system consisting of a transfer plasmid containing the LTR sequence, packaging signal, an internal promoter and the transgene of interest as well as a packaging plasmid encoding the GAG and POL proteins, another plasmid encoding REV and lastly a plasmid encoding an ENV protein. In a variation, the envelope protein may be the G protein from vesicular stomatitis virus (VSVG). The ENV protein, including but not limited to the VSVG, may confer tropism.

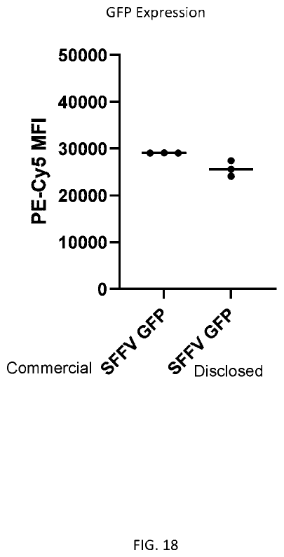

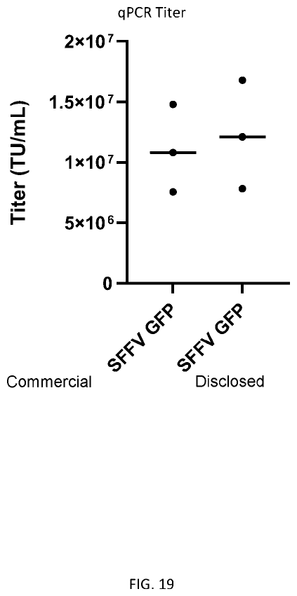

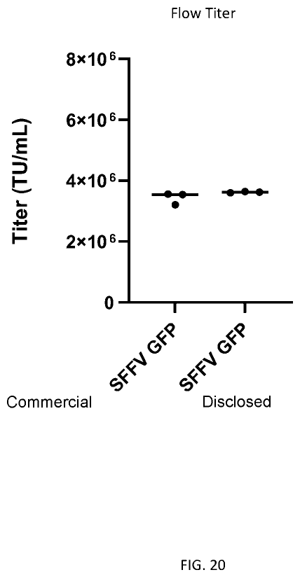

The currently disclosed lentiviral vector system has been optimized to address drawbacks of conventional lentiviral vector systems. Among others, the system reduces the likelihood of recombination to replication-competent retrovirus (RCR), impedes mobilization of vector RNA in the case of RCR superinfection, increases the autonomy and reduces competition for transcription factors of promoters driving the transcription of the proviral RNA and accessory plasmid coding DNA sequences within cells co-transfected with the plasmid system and theoretically reduces the risk of insertional upregulation of neighboring alleles depending on the choice of the internal enhancer/promoter. The disclosed lentiviral vector also decreases the chance of expression of, e.g., out of frame polypeptides, out of context polypeptides, non-native human polypeptides, HIV polypeptides, non-native HIV polypeptides, as well as non-naturally occurring polypeptides. The disclosed lentiviral vector system achieves these features without comprising the potency or expression of the integrated transgene allele as compared to commercially available and conventional systems. (See data at FIG. 18, FIG. 19, FIG. 20, and so on discussed herein below.)

The disclosed lentiviral vector system thus decreases the time and intensiveness of regulatory evaluation. The disclosed lentiviral system also increases the efficacy of the system, e.g., when used as a gene therapy platform. Some current gene therapy interventions display poor durability, including a lack of effectiveness over time. The disclosed lentiviral vector system decreases at least one potential cause of decreased efficacy in the nature of increased immune clearance of therapeutics due to aberrant gene products, including natural or unnatural products and also including HIV proteins. Production of any aberrant polypeptides can lead to decreased efficacy.

The disclosed lentiviral system improves safety as compared to conventional lentiviral systems without substantively reducing efficacy, e.g., while maintaining approximately comparable titers with those conventional lentiviral systems.

### Reduction of RCR/RCL

Currently there have been no discoverable reports of Replication competent retrovirus (RCR) or replication competent lentivirus (RCL) being detected in third or fourth generation lentiviral products. Despite this recent history of safety, evidence of RCR/RCL has been documented in preclinical studies using first generation vectors and FDA guidance still recommends RCR/RCL testing on all vector production lots, ex vivo transduced cell products and specific patient samples out to 15 years post administration. From a sponsor perspective, this is a labor and time intensive endeavor which can take as much as a month to complete. We disclose a system that reduces RCR/RCL events by, among other things, introducing reduced homology between the plasmids that make up the system, introducing multiple stop codons. This is achieved as discussed below.

### Insertional Mutagenesis

The disclosed lentiviral system reduces incidence of insertional mutagenesis. Insertional mutagenesis is the process by which insertion of a retroviral vector into the host cell genome alters endogenous gene expression that leads to pathogenic consequences including cellular transformation and cancer development. Potential pathogenic consequences can include retroviral integration adjacent to proto-oncogenes, leading to their upregulated expression. We disclose a system that reduces insertional mutagenesis by, among other things discussed herein, reduced homology between the plasmids that make up the system. This is achieved as discussed below.

### Vector Insertion Mediated Mutagenesis

Many types of vector insertion-mediated mutagenesis that have been described clinically which may include but are not limited to enhancer insertion, promoter insertion, insertional inactivation, and activation by 3′ end truncation. We disclose a system that reduces vector insertion mediated mutagensis events by, among other things, reduced homology between the plasmids that make up the system. This is achieved as discussed below.

### Self Inactivating (SIN) Vector

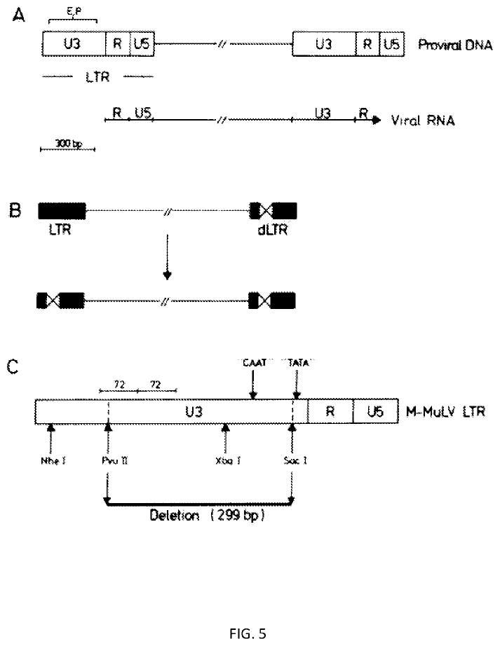

SIN vector design involves removal of critical transcriptional regulatory sequences in the U3 region of the 3′ LTR, which as shown in FIG. 5 may be used as a template to recreate the 5′ U3 sequence in the 5′ LTR of the DNA viral genome, which is lost during production of the viral RNA genome due to transcription beginning about 600 bp downstream of the beginning of the LTR. (See, e.g., S F Yu et al., Self-inactivating retroviral vectors designed for transfer of whole genes into mammalian cells. PNAS. 1986, 83 (10) 3194-3198, incorporated herein by reference in its entirety). As the U3 region is useful for the enhancer and promoter activity of retroviral LTRs, SIN vectors result in the integration of a proviral genome with both U3 regions deleted and thus no promoter/enhancer activity. In variations, an internal promoter may be utilized to drive expression of the transgene. Most known promoters are believed to have significantly less enhancer activity than the viral LTRs. Decreased enhancer activity may reduce the risk of insertional mutagenesis through enhancer activity. In a variation, additional protection may be achieved through the inclusion of insulator sequences that theoretically can further reduce the risk altering the expression of nearby genes.

### Chemistry and Manufacturing Controls

Retroviral vectors are one of, if not, the most complicated biotherapeutic platform under development. Therefore, a continuing struggle has been vector manufacturing consistency and characterization during clinical development and commercialization. We disclose herein a lentivector system that implements critical advances in both the technical aspects of vector design as well as the manufacturing processes and bioanalytical methods used to characterize clinical vectors. Addressing vector identity, the disclosed lentiviral system was created through custom DNA synthesis.

### Disclosed Vector

We disclose a lentiviral vector and construct which can be utilized to introduce expressible polynucleotide sequences of interest into host cells. The lentivirus vector and construct may be pseudotyped with an engineered or native viral envelope protein from another viral species, including non-lentiviruses, which may alter the host range and infectivity of lentivirus vector system. The disclosed lentiviral vectors can be used in, for example but not limited to, protein production (including vaccine production), gene therapy, delivery of therapeutic peptides, delivery of siRNA, ribozymes, ant-sense, and other functional polynucleotides, etc.

We disclose vectors comprising the nucleic acids, transcription control units, promoters and fragments thereof, optimized genes, and expression constructs disclosed herein. The vector may be of any type, for example, it may be a plasmid vector or a plasmid, baculovirus, stably expressing producer cell line, mRNA.

The efficacy of therapy is, in general, dependent upon adequate and efficient delivery of the donated transgene. This process is usually mediated by viral vectors. As such, the invention provides viral vectors, which may be based, for example, on the herpes simplex virus, adenovirus, or lentivirus. The viral vector may be a lentivirus vector or a derivative thereof, including but not limited to the HIV HXB2.

In a variation, the viral vector comprises a lentivirus vector from a naturally derived serotype or isolate, or a derivative thereof. In a further variation the disclose viral vector comprises a self-inactivating (SIN) lentivirus production system designed for delivery of therapeutic nucleic acids in, e.g., a clinical setting, manufacturing, or research setting. In a further variation the disclose viral vector comprises a self-inactivating (SIN) lentivirus production system designed with reduced homology between the plasmids that make up the system. In a further variation the reduced homology between the plasmids that make up the system is achieved by using alternative promoters, introns, and polyA combinations.

In a further variation the reduced homology between the plasmids that make up the system is achieved by using on each plasmid a unique kanamycin antibiotic resistance gene. By unique, it is meant that each kanamycin resistance gene has a different sequence from each of the other kanamycin resistance (KanR) genes. In a further variation, the kanamycin resistance genes include at least four variations in coding sequence. In a further variation, the KanR gene sequences are chosen from a liver codon optimized KanR gene, a hCD8 codon optimized KanR gene, a conventional KanR gene, and a NK Cell codon optimized KanR gene.

In a variation, we provide human CD8 cell codon optimized kanamycin resistance gene SEQ ID NO: 7. In a further variation, we provide human NK cell codon optimized kanamycin resistance gene SEQ ID NO: 16. And in a further variation, we provide liver codon optimized kanamycin resistance gene SEQ ID NO: 23. A conventional KanR gene is provided at SEQ ID NO: 33.

The disclosed lentiviral vector system provides novel 293T cell codon optimized VSVG at SEQ ID NO: 19 and novel 293T cell codon optimized REV at SEQ ID NO: 36. Codon optimization increases the expression of VSVG and REV respectively when expressed in 293T cells. This is unique as most known and/or conventional systems use the standard approach of optimization for human expression using the pan-genome codon usage in the human genome. It is a unique approach to optimize for expression specifically in 293 cells using proprietary tissue specific codon optimization approach previously described (see, e.g., Brown, H. C., Zakas, P. M., George, S. N., Parker, E. T., Spencer, H. T., & Doering, C. B. (2018). Target-Cell-Directed Bioengineering Approaches for Gene Therapy of Hemophilia A. Molecular Therapy—Methods & Clinical Development, 9, 57-69. https://doi.org/10.1016/j.omtm.2018.01.004, incorporated herein by reference in its entirety.)

In a variation, multiple copies of each stop codons have been introduced to each reading frame (sense and antisense) to arrest translation of unwanted genes. Multiple stop codon strategy has been employed to at least the GAG/POL plasmid and the transfer plasmid of the disclosed lentiviral system but it should be understood that the same technique may be applied to other plasmids in the system.

In a further variation, multiple copies of each stop codons have been introduced to each reading frame (sense and antisense) to arrest translation of for example but not limited to HIV-1 vif, antisense proteins, integrase or Asp in this region before it can, e.g., result in a mutant C terminus. In a further variation, multiple copies of each stop codon have been introduced into each reading frame (sense and antisense) to reduce the chances of mutant HIV-1 vif and antisense protein expression in the GAG/POL plasmid of the system. In a further variation, a six frame stop element (SEQ ID NO: 6) has been inserted between the integrase and the RRE elements on the GAG/POL plasmid. This six frame stop element (SEQ ID NO: 6) provides insulation between the integrase and RRE elements. The six frame stop element (SEQ ID NO: 6) may be inserted on both the upstream and downstream portions. The six frame stop element also reduces the potential of translational read through between the integrase and RRE elements. By reducing the potential of translational read through the six frame stop element (SEQ ID NO: 6) reduces the potential for production of aberrant protein products. In a variation, this SEQ ID NO: 6 element provides all three stop codons in all six reading frames to reduce the potential of aberrant protein products. Potential aberrant protein products avoided may include but are not limited to NEF.

In a variation, a six frame stop element (SEQ ID NO: 25) has been inserted between on the transfer plasmid. For example, a six frame stop element (SEQ ID NO: 25) has been inserted on both the 5′ and the 3′ ends of the transgene insertion site. This six frame stop element (SEQ ID NO: 25) provides insulation between to prevent translational readthrough. By reducing the potential of translational read through the six frame stop element (SEQ ID NO: 25) reduces the potential for production of aberrant protein products. By using SEQ ID NO: 25, the region the therapeutic promoter/cDNA would be inserted into is insulated with multiple copies of each stop codon in all reading frames on both the 5′ and 3′ termini of the cloning site where the therapeutic cassette will be inserted (e.g., Agel/SgrAI). This will again arrest any unwanted translational readthrough of either viral or therapeutic proteins in either direction. This is unique as compared to most known and/or conventional vectors which use either cloning plasmid inserts, random sequences, or multiple cutting sites in this region to provide spacing between the transgene and functional viral elements. The disclosed lentiviral system transfer plasmid used this spacer more constructively by inserting SEQ ID NO: 25 on each of the 5′ and 3′ termini of the cloning site.

In a further variation the reduced homology between the plasmids that make up the system is achieved by using on each plasmid a unique promoter. This is a diversion from the traditional practice of using the CMV promoter on each of the plasmids that make up a viral vector system. Many known recombinant lentiviral production systems use the CMV promoter to drive expression from all primary plasmids in the system, for example, if GAG/POL and REV are separated onto separate plasmids, known or conventional systems use CMV promoters to drive all four plasmids in the system. If GAG/POL and REV are on the same plasmid, known or conventional systems use CMV promoters to drive all three plasmids in the system. By contrast, the disclosed lentivirus vector system uses a unique promoter to drive each of at least three of the four plasmids in the system. By unique, it is meant that each promoter sequence found on a plasmid has a different sequence from each of the other promoter sequences found on the other plasmids of the system.

The novel three or more unique promoter design decreases promoter competition. Promoter competition is a phenomenon by which multiple copies of the same promoter depletes the cellular supply of cytosolic transcription factors since they all have the same binding sites. This leads to decreased expression. The disclosed system includes at least one or more of the CMV promoter (SEQ ID NO: 9), the Ubiquitin C (UbC) promoter (SEQ ID NO: 35), the CD68 promoter (SEQ ID NO: 2, SEQ ID NO: 46, and others disclosed), the PGK promoter (SEQ ID NO: 18); and the EF1a promoter (e.g., SEQ ID NO: 29, SEQ ID NO: 45). In a non-limiting variation, the REV plasmid uses Ubiquitin C promoter, the VSVG plasmid uses the PGK promoter, the GAG/POL plasmid uses the CMV promoter, the transfer plasmid uses one of the EFla promoter, the CD68 promoter, or the CMV promoter.

In a variation, each plasmid of the system uses a distinct and unique poly adenylation signal. For example, the system includes at least one or more of the Beta globin polyA (SEQ ID NO: 38), PGK polyA (SEQ ID NO: 10), human growth hormone polyA (SEQ ID NO: 21), and the bovine growth hormone polyA (SEQ ID NO: 64). In a further variation, the REV plasmid of the disclosed lentiviral system comprises the Beta globin polyA signal (SEQ ID NO: 38); the GAG/POL plasmid comprises the PGK polyA (SEQ ID NO: 10); the transfer plasmid uses the bovine growth hormone polyA (SEQ ID NO: 64); and the VSVG plasmid uses the human growth hormone polyA (SEQ ID NO: 21). Together, the disclosed changes remove over 1,900 bases of homologous sequence from each plasmid as compared to unmodified plasmids containing comparable base genetic structure.

In a variation, the lentiviral vector provides the functional DNA elements in at least about four (4) plasmids. The at least about four plasmids include but are not limited to a transgene transfer plasmid, an ENV expression plasmid, a REV expression plasmid, and a GAG/POL expression plasmid. In a further variation, the ENV expression plasmid may be a VSVG expression plasmid.

The disclosed lentiviral vector is capable of producing synthetic lentiviral particles with an enhanced safety profile as compared to first- and second-generation lentiviral particles. Enhancing safety of the GAG/POL is not typically addressed because the GAG/POL plasmid of the lentiviral vector system is technically not transferred to a patient. Therefore, the changes and the approach of modifying the GAG/POL plasmid is a unique approach. The approach is unique also in that it is not intuitive since the lentiviral GAG/PIL plasmid is technically not transferred to the patient. If the lentiviral vector system is used in gene therapy applications, aberrant production of a transmembrane protein from the GAG/POL plasmid could theoretically be transferred during the budding process which would potentially deliver that protein to cells. An example of a transmembrane protein that could be transferred during the budding processing includes but is not limited to HIV antisense protein (ASP). Therefore, this unique modification represents an improvement over existing systems.

The disclosed lentiviral vector system may be used for any gene transfer application, including but not limited to in the clinical, manufacturing, or research setting. By reducing the homology between the plasmids that make up the lentiviral vector system, there is a reduced threat of homologous recombination between the plasmids in the lentiviral vector system. This further reduces the threat of aberrant recombination which may lead to unintentional production of replication competent virus. The disclosed lentiviral vector system produces high titer vector while incorporating novel features such as those listed above to reduce homology between plasmids and prevent translation of, e.g., unwanted protein products including but not limited to HIV protein products, native protein products, non-native natural protein products and/or non-natural protein products.

While the lentiviral system is discussed, it should be understood that the disclosed system of reducing homology between plasmids and the genes disclosed herein may be applied to the field of AAV gene therapy. The skilled person can select an appropriate serotype, clone or isolate of virus for use with the presently disclosed features including the presently disclosed gene sequences. It will be understood that this disclosure encompasses other virus and serotypes that have yet been identified or characterized.

### Transfer Vector

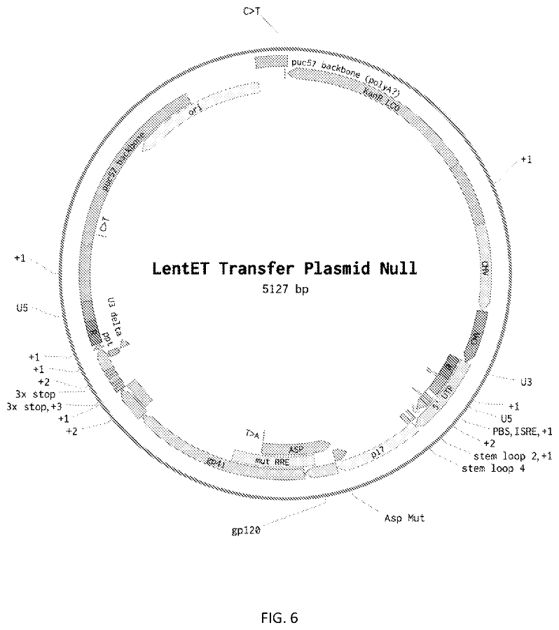

Turning to FIG. 6, we disclose a lentiviral vector system including a novel lentiviral transfer vector. A transfer vector is a construct which contains the polynucleotide sequences which are packaged into the transducing lentiviral vector. The Transfer vector sequence corresponding to FIG. 6 is provided as SEQ ID NO: 1 (with a fVIII transgene); SEQ ID NO: 22 (with no transgene); and SEQ ID NO: 28 (with a fVIII transgene).

FIG. 6 demonstrates an “empty” transfer plasmid, meaning that FIG. 6 demonstrates the basic transfer plasmid that does not include a transgene cDNA. Any desireable transgene cDNA and/or transgene and promoter can be inserted into the lentiviral vector system transfer plasmid for integration into a host cell. The system is agnostic to any particular sequence of cDNA, internal promoter, or transgene cassette.

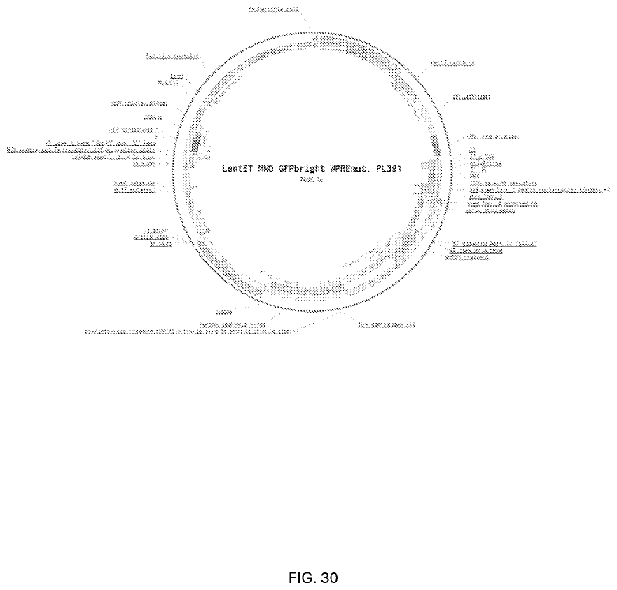

FIG. 30 demonstrates a second transfer plasmid that includes transgene DNA for GFPbright. It should be understood that the inclusion of the GFPbright sequence is for illustrative purposes only. Any desireable transgene cDNA and/or transgene and promoter can be inserted into the lentiviral vector system transfer plasmid for integration into a host cell. The system is agnostic to any particular sequence of cDNA, internal promoter, or transgene cassette.

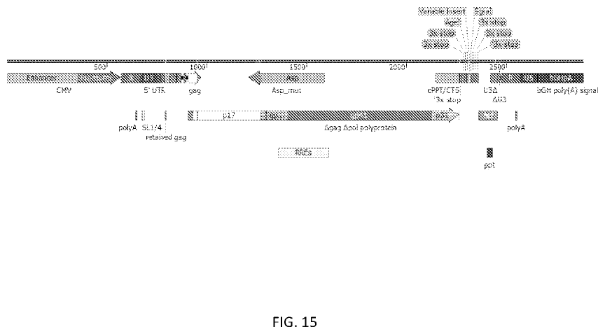

Since the basics of lentiviral vector systems are well known, we will focus our description on the novel elements of our system. For example, it should be understood that any suitable lentiviral 5′ LTR sequence, packaging sequence (psi), 3′ LTR, U3 region elements, can be placed in the transfer vector. The transfer vector can further include other additional elements, e.g., arranged in any order (with the already described elements): 5′ LTR, PBS, packaging sequence, splice donor (SD), origin of replication, optionally a central polypurine tract (PPT), RRE, MCS, splice acceptor (SA), and a modified minimally functional 3′ LTR. The expressible heterologous polynucleotide sequence can be inserted in the “variable insert” site as indicated in FIG. 15. In that variation, AgeI, SgrAI, and NotI restriction sites are present to facilitate specific, directional cloning into this site.

The transfer vector can also contain one or more SD (naturally-occurring or modified) sites. Such sequence can be intact and fully native, or it can be modified by any method known or hereafter discovered.

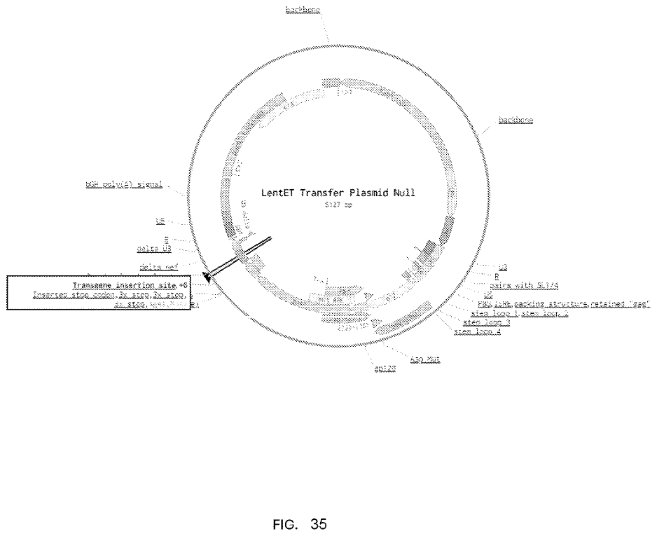

The disclosed lentiviral vector system transfer plasmid carries the minimal viral elements useful to permit packaging of the RNA product into lentiviral capsids. While the RNA product may be driven by any promoter, including any one of the promoters disclosed herein, FIG. 6 illustrates a transfer plasmid where the RNA product is driven by an external CMV promoter. Similarly, while the RNA product may be terminated by any polyA, including any polyA disclosed herein, FIG. 6 shows a bovine growth hormone (bGH) polyA signal (SEQ ID NO: 64). FIG. 30 illustrates a second transfer plasmid where the RNA product is driven by a MND promoter. Similarly, while the RNA product may be terminated by any polyA, including any polyA disclosed herein, FIG. 30 shows a bovine growth hormone (bGH) polyA signal (SEQ ID NO: 64). FIG. 35 shows the Transgene insertion site.

FIG. 35 is a plasmid map of an empty LentET transfer plasmid, i.e., it is not carrying a transgene. The transgene insertion site (highlighted) is flanked by triple stop translation readthrough insulator sequences. The transgene plasmid is inserted using AgeI (5′) and NotI (3′) restriction sites. For example, LentET MND GFP (FIG. 30) is created by inserting the MND (internal promoter) and GFP (coding sequence) using the Agel/NotI restriction sites, creating the LentET MND GFP transfer plasmid

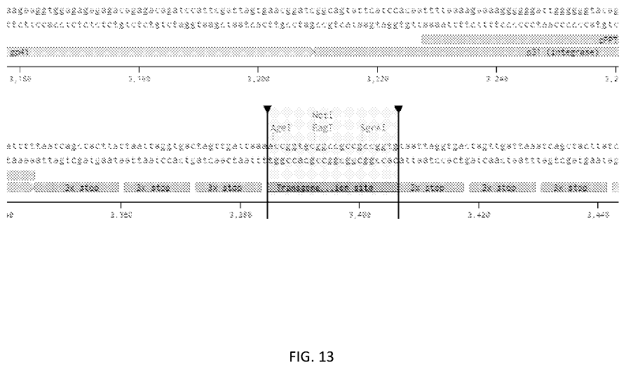

In a variation, the lentivirus vector system transfer plasmid may include at least one of a) transgene cDNA, b) one or more Agel/NotI/SgrAI restriction sites restriction handles, c) KanR gene (which may be a codon optimized KanR gene, which may further be a liver codon optimized KanR gene or such other KanR genes disclosed herein or known), d) mutated RRE portion, e) six frame triple stop codon at one more of the 5′ and 3′ side of the transgene insertion site, f) integrase stop codon, g) promoter selected from CMV or EF1a. In a further variation, the lentivirus vector system transfer plasmid may include at least one of a) transgene cDNA, b) one or more AgeI/NotI/SgrAI restriction sites restriction handles, c) KanR gene (which may be a codon optimized KanR gene, which may further be a liver codon optimized KanR gene or other KanR gene disclosed herein or known e.g. a gene encoding SEQ ID NO: 34) (e.g., SEQ ID NOS: 7, 16, 23, 33), d) mutated RRE portion (SEQ ID NO: 24), specifically a mutation added at position 2672 of the RRE or its equivalent when aligned with the RRE of SEQ ID NO: 22, e) six frame triple stop codon (e.g., SEQ ID NO: 25) at one more of the 5′ and 3′ side of the transgene insertion site (SEQ ID NO: 26) (See FIG. 13), f) integrase stop codon (SEQ ID NO: 27), which may be located directly after P31/integrase, g) promoter selected from CMV (SEQ ID NO: 9) or EF1a (SEQ ID NO: 29) or others disclosed herein.

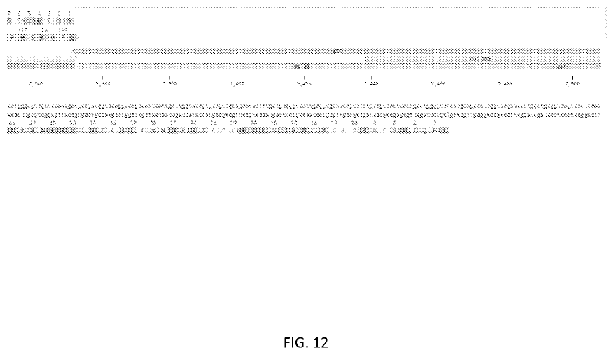

FIG. 12 illustrates the potential protein products that are avoided by the disruption introduced by SEQ ID NO: 24. In this case, a theoretical protein product (designated gp120 on FIG. 12) that is a synthetic, out of frame, unnatural protein may be avoided. FIG. 13 illustrates the P31 integrase with terminate stop codon (SEQ ID NO: 27). This was inserted to remove any possibility of read through. Although this is not predicted to be a translated region, the stop was added out of an abundance of caution. SEQ ID NO: 29 is a EF1a promoter, which contains a single base pair mutation. The single base pair mutation removes a restriction site, making cloning the transgene into the plasmid easier. Another EF1a promoter sequences are provided at SEQ ID NO: 45.

FIG. 15 provides another view of a lentivirus transgene design. In this FIG. 15, the elements have the following meaning: FIG. 15 CMV: wild type genomic human betaherpesvirus 5 isolate UCSF-1a (CMV) enhancer/promoter; Spacer: None, CMV promoter leads directly into HIV genomic sequence; 5′ UTR: 100% genomic HIV-1 genomic sequence from R to gag p17 start codon; P17: Begins at its start codon and is truncated slightly on the 3′ end where it hits gp120, Coding sequence begins with native ATG. The plasmid includes an inserted “cg” doublet which shifts reading frame and prematurely terminates p171, the 5′ end contains final stem loop of psi region, unsure of function from the rest; gp120/41: Picks up RRE sequence as well as the HIV antisense protein (Asp), A stop codon is added to prevent translation of the full (mutant) antisense protein; gp41: Extended to be sure entire RRE is contained, functional area is not precisely defined; p31: Picks up central polypurine site; 3× stops: Spacer sequence flanking expression cassette, Provides stop codons in all 6 reading frames, cDNA is cloned in Xhol/NotI, promoters are cloned in Agel/Xhol; 3′ UTR: U3 is deleted comparably to other designs; PolyA: Bovine growth hormone polyA (not packaged) (SEQ ID NO: 64); Backbone: Puc57 kan-based, uses arbitrarily codon optimized KanR cDNAs to reduce plasmid homology.

It will be understood by those of skill in the art that the cDNA is not limited to a therapeutic product or coding DNA sequence. The disclosed lentiviral vector system may be incorporated with CRISPR, shRNA, and other therapeutic, commercial, or research strategies now known or discovered after.

### Accessory Constructs

We disclose a lentiviral system which includes accessory constructs (e.g., a plasmids or isolated nucleic acids). Such constructs contain the elements that are useful for producing a functional lentiviral transduction vector in a compatible host cell, and packaging into it an expressible heterologous sequence. These elements include structural proteins (e.g., the gag precursor), processing proteins (e.g., the pol precursor), such as proteases, envelope protein, and the expression and regulatory signals needed to manufacture the proteins in host cells and assemble functional viral particles. Although the embodiment described below contains the envelope and gag-pol precursor on different plasmids, they can be placed on the same plasmid, if desired, or can be further divided to include separate plasmids for each of the gag, pol, and envelope proteins.

In a variation, the lentiviral accessory plasmid can comprise one or more of the following elements in any suitable order or position, e.g., a) lentivirus 5′ LTR comprising a functional native promoter operably linked to a polynucleotide sequence coding for lentivirus gag and pol (e.g., a lentivirus gag-pol precursor); and b) heterologous promoter operably linked to an envelope coding sequence. The lentivirus 5′LTR can optionally contain heterologous enhancer sequences located upstream from the native sequence.

It will be understood by one of skill in the art that any suitable lentiviral 5′ LTR can be utilized in accordance with the present invention, including an LTR obtained from any lentivirus species, sub-species, strain or clade. This includes primate and non-primate lentiviruses.

### GAG/POL

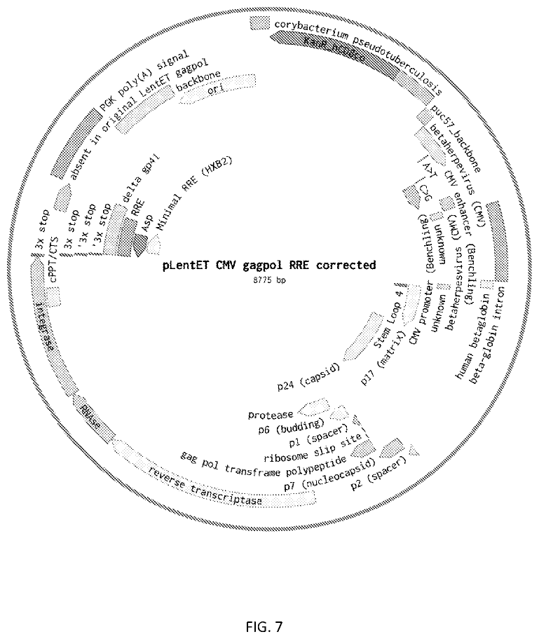

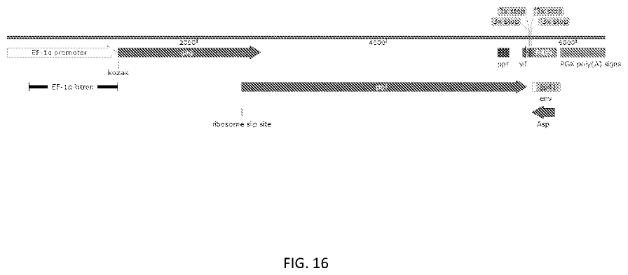

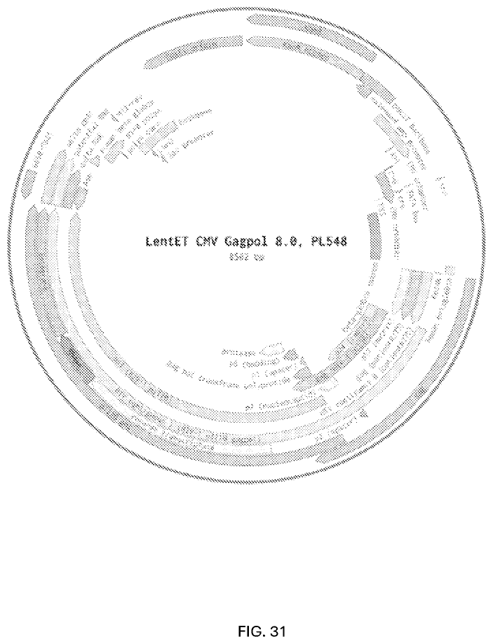

The lentiviral vector GAG/POL plasmid may provide many of the lentiviral proteins useful to produce and package lentiviral particles. In a variation, it may be drive by any promoter, including but not limited to promoters disclosed herein. FIG. 7 shows an exemplary GAG/POL plasmid of the disclosed lentiviral vector system. In that example, the GAG/POL plasmid is driven by a CMV promoter. FIG. 16 shows another view of the GAG/POL plasmid. FIG. 31 provides the third generation GAG/POL plasmid (correlating to SEQ ID NO: 43). GAG/POL plasmid sequences are provided at the following SEQ ID NOS: SEQ ID NO: 5, SEQ ID NO: 43.

Native Gag-Pol sequences can be utilized in the accessory vector, or modifications can be made. Examples of possible modifications include but are not limited to, chimeric Gag-Pol, where the Gag and Pol sequences are obtained from different viruses (e.g., different species, subspecies, strains, clades, etc., and/or where the sequences have been modified to improve transcription and/or translation, and/or reduce recombination. In other embodiments of the present invention, the sequences coding for the gag and pol precursors can be separated and placed on different vector constructs, where each sequence has its own expression signals.

It will be understood by one of skill in the art that additional promoter and enhancer sequences can be placed upstream of the 5′ LTR in order to increase, improve, enhance, etc., transcription of the gag-pol precursor. Examples of useful promoters, include, mammalian promoters (e.g., constitutive, inducible, tissue-specific), CMV, RSV, LTR from other lentiviral species, and other promoters as mentioned above and below.

Safety is increased with such vectors as there is no possibility that transcriptional readthrough would result in a RNA that contains both functional gag-pol and envelope sequences. Transcriptional read through can be prevented by utilizing strong polyadenylation sequences that terminate transcription.

Turning again to FIG. 7, the encoded protein elements are p17 (HIV matrix), p24 (HIV capsid), p7 (HIV nucleocapsid), HIV P1, HIV P6, HIV protease, HIV reverse transcriptase, RNAse, and integrase also carries a rev responsive element in the 3′ UTR. It is driven by a mutated CMV promoter (SEQ ID NO: 9) which is linked to a beta-globin intron (SEQ ID NO: 9) and terminated by a PGK polyadenylation signal (SEQ ID NO: 10). Use of the PGK polyA signal (SEQ ID NO: 10) in combination with the CMV Beta-globin promoter (SEQ ID NO: 9) is a unique combination not otherwise used.

As discussed above, the GAG/POL plasmid shown on FIG. 7 includes a six frame stop element (SEQ ID NO: 6) located between the integrase and the RRE elements. It also includes a unique hCD8 codon optimized KanR gene (SEQ ID NO: 7).

### Poly A

In addition, any of the plasmids in the disclosed lentivirus vector system can further comprise transcription termination signals, such as a polyA signal that is effective to terminate transcription driven by the promoter sequence. Any suitable polyA sequence can be utilized, e.g., sequences from beta globin (mammalian, human, rabbit, etc), thymidine kinase, growth hormone, SV40, and many others. The use of unique polyA signals improves safety by further reducing homology between plasmids.

### ENV

The disclosed lentiviral vector construct can further comprise an accessory plasmid which is an envelope module comprising a heterologous promoter operably linked to an envelope coding sequence. The envelope polypeptide is displayed on the viral surface and is involved in the recognition and infection of host cells by a virus particle. The host range and specificity can be changed by modifying or substituting the envelope polypeptide, e.g., with an envelope expressed by a different (heterologous) viral species or which has otherwise been modified. This is called pseudotyping. See, e.g., Yee et al., Proc. Natl. Acad. Sci. USA 91:9564-9568, 1994. Vesicular stomatitis virus (VSV) protein G (VSV G) has been used extensively because of its broad species and tissue tropism and its ability to confer physical stability and high infectivity to vector particles. See, e.g., Yee et al, Methods Cell Biol., (1994) 43:99-112

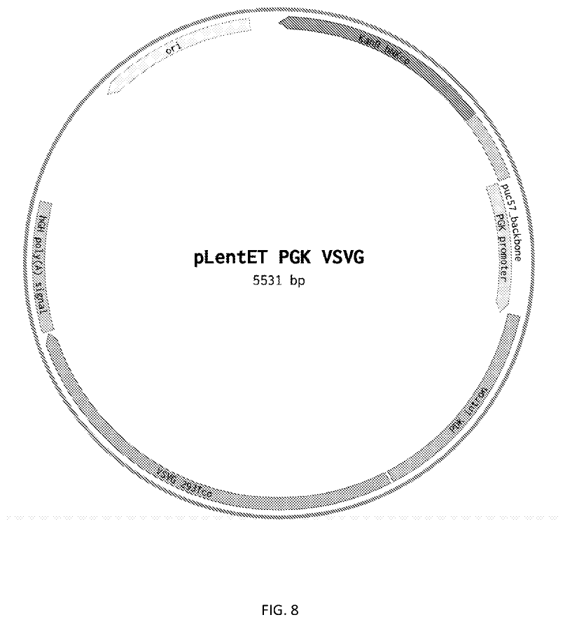

Turning to FIG. 8, we disclose a viral envelope (ENV) protein VSVG that is codon optimized for better expression in 293T producer cells. While any promoter can be used, as discussed herein, the ENV plasmid of FIG. 8 is driven by a PGK promoter linked to a PDK intron and terminated by a human growth hormone polyadenylaton signal (SED ID NO: 21). The combination of the PGK intron/PDK promoter and the VSVG gene is unique to the disclosed lentiviral system. The PGK promoter is a unique choice to drive VSVG as compared to the most conventional choice is the CMV promoter. The CMV promoter is more powerful and is thought to be the optimal promoter to drive expression. The PGK promoter is not as strong as the CMV promoter and thus its effectiveness gave surprising results. The VSVG coding sequence (SED ID NO: 19) was codon optimized using a unique strategy tailored specifically for expression in 293T cells. This was done using a technique we call “tissue specific codon optimization”, which seeks to mimic the codon usage bias of genes highly and specifically expressed in the target tissue/cell. (See Brown 2018.)

The lentiviral vector system ENV plasmid variation shown in FIG. 8 includes a codon optimized KanR gene that is unique from that used on other plasmids and that is codon optimized for hNK tissue specific expression (SEQ ID NO: 16). This further reduces homology between the plasmids of the lentiviral vector system.

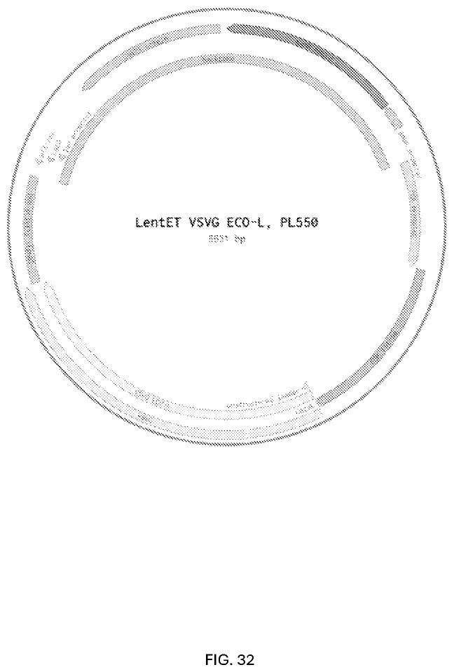

A third generation ENV plasmid is provided at FIG. 32. It is correlated with SEQ ID NO: 42. It has a further modified PGK Promoter of SEQ ID NO: 51.

### REV

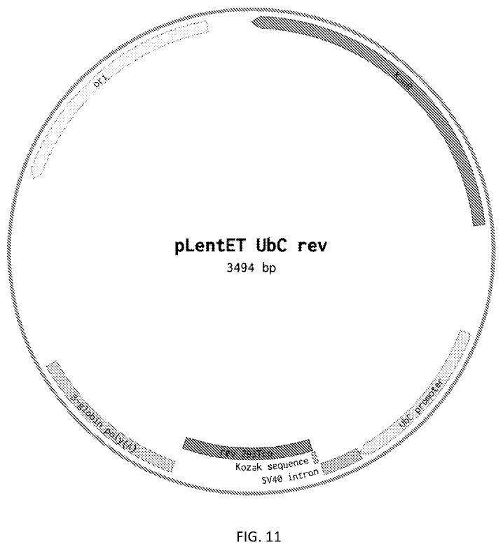

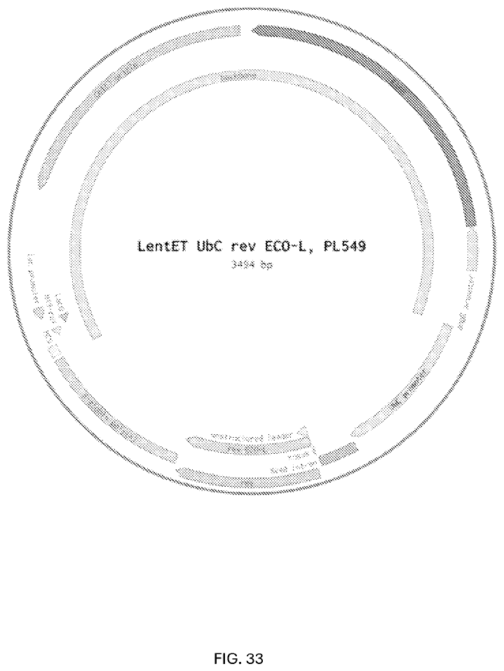

The REV plasmid encodes the HIV rev protein. REV binds the rev-responsive element and assists in nuclear export of the mRNA. One exemplary REV plasmid is shown in FIG. 11. It is driven by a ubiquitin C (UbC) promoter linked to an SV40 intron (SEQ ID NO: 35) and terminated by a beta globin polyadenylation signal (SEQ ID NO: 38). It is unique to use the UbC promoter to drive the REV gene. The REV gene (SEQ ID NO: 36) disclosed in the REV plasmid has been codon optimized for maximum expression in HEK 293T cells. A third generation REV plasmid is shown at FIG. 33, corresponding with SEQ ID NO: 41.

The REV plasmid of one disclosed system includes, among other elements, a KanR gene shown in SEQ ID NO. 33, which is uniquely different in sequence from the KanR nucleotide sequences on the other plasmids in the lentiviral vector system. This further reduces the sequence homology between plasmids and increases the safety profile of the lentiviral vector as described above. The combination of the UbC promoter and the SV40 intron (SEQ ID NO: 35) with the REV gene is unique to this disclosed system. Furthermore, the combination of the Beta globin polyA (SEQ ID NO: 38) with the UbC promoter and/or the PGK/SV40 intron and/or with the REV gene is also unique to this lentiviral vector system.

The REV plasmid of the third generation disclosed system includes, among other elements, a KanR gene shown integrated into SEQ ID NO. 41, which is uniquely different in sequence from the KanR nucleotide sequences on the other plasmids in the lentiviral vector system. This further reduces the sequence homology between plasmids and increases the safety profile of the lentiviral vector as described above. The combination of the UbC promoter and the SV40 intron (inserted into SEQ ID NO: 41) with the REV gene is unique to this disclosed system. Furthermore, the combination of the Beta globin polyA (inserted into SEQ ID NO: 41) with the UbC promoter and/or the PGK and/or SV40 intron and/or with the REV gene is also unique to this lentiviral vector system.

While an exemplary arrangement is provided above, it will be understood that the promoter, intron, polyA, and kanamycin genes can likely be substituted between plasmids. We have put effort into finding the optimum choice/arrangement of these elements but finding other combinations of these elements linked to coding/functional regions is within the spirit of the disclosure.

The exact interaction of the elements is very complex and still a subject of research in the HIV field. Broadly, the system functions as follows:

The 4 plasmids are co-transfected into lentivirus producer cells, often, but not exclusively, an HEK 293 cell line. After some number of hours, these plasmids are expressed in the producer cells

The VSVG plasmid drives expression of the VSVG transmembrane protein molecule. This molecule is trafficked to the surface of the producer cells.

The REV plasmid drives expression of the REV protein.

The GAG/POL plasmid drives expression of the multiple gene products coded in it. Gag and pol are produced as single polyprotein amino acid chains using an internal ribosomal slip site to change reading frames between gag and pol genes. These proteins are the structure and enzymatic proteins that allow the formation and function of the lentiviral particle.

The promoter, e.g., the CMV or EF1a promoter, driving the transfer plasmid drives expression of the RNA molecule that will be incorporated into the lentiviral molecule. This RNA includes the HIV UTRs that will assist in packaging and, in therapeutic/functional designs, an internal promoter/cDNA combination.

The REV protein product binds the REV-responsive element in the transgene RNA product and assists in nuclear export

Aided by the RRE element, which acts as a scaffold, the lentiviral molecule is formed in a manner analogous to how HIV is formed.

The forming lentiviral molecule buds off of the producer cell, where it picks up the VSVG membrane proteins present on the producer cell.

These molecules are now capable of transducing and integrating into a target cell, e.g., those expressing the LDLR.

### Transgenes

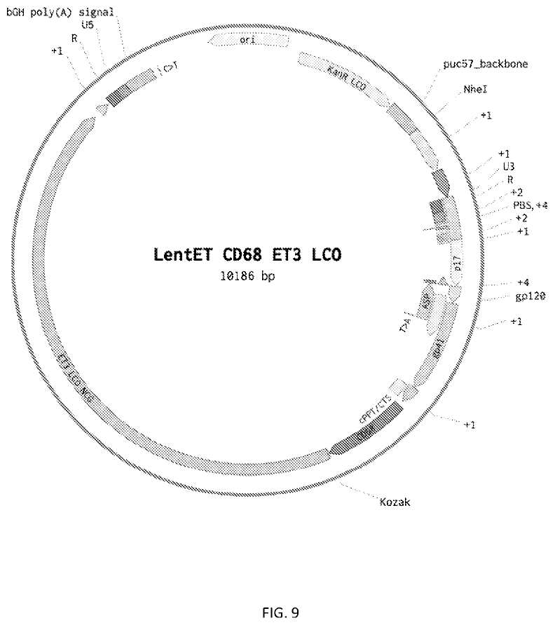

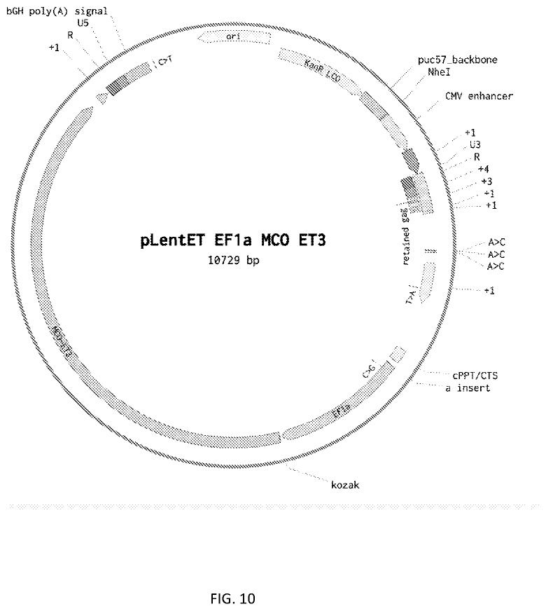

We provide sequences and vectors that demonstrate the use of various fVIII constructs in the disclosed lentivirus vector system. For example, FIG. 9 shows the disclosed lentivirus system operatively linked to a liver codon optimized fVIII gene. FIG. 10 shows the disclosed system operatively linked to a myeloid codon optimized fVIII gene. As discussed above, the lentiviral vector system is agnostic to the gene product. While fVIII is described, transgenes successfully packaged in this system include but are not limited to GFP, UNC13D, EGFP, insulin, and others.

### Method of Producing

An exemplary method of producing a lentivirus vector system includes the following protocol, day by day. This is only one example and various modifications or alternative methods may be used.

The plasmids themselves may be commercially synthesized and delivered as a usable product. The are used as follows, which details how the lentivector is produced in a 6-well plate format:

293T-17 cells were grown to ~70% confluency in tissue culture treated plates

Plasmids were transfected into cells using PEI at a ratio of luL PEI to lug of plasmid

Media was changed on cells 24 hours after transfection

Conditioned supernatant was collected at 48 and 72 hours post transfection

Pooled supernatant was filtered through 0.45 μM PVDF filters

Vector was titered on 293T-17 cells. Titer was determined 5 days post transduction by flow cytometry (GFP vectors) or qPCR directed against the RRE region of the lentiviral transgene cassette (GFP and FVIII vectors) against a standard curve generated from the appropriate transgene plasmid.

### EXAMPLES

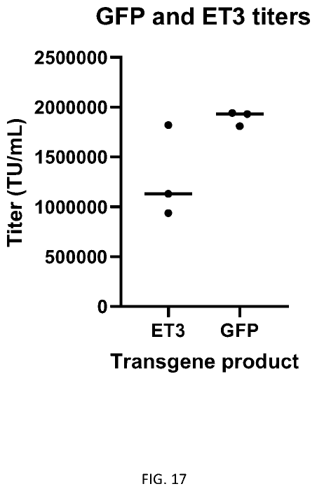

Using the above described procedure, we achieved the infection titers shown in FIG. 17 using the lentiviral vector system. These vectors were produced and titered on HEK 293T-17 cells. Titers were performed using quantitative PCR using primers directed against the RRE region of the transgene plasmid. In FIG. 17, this data shows the system produces high titer, functional vector particles. “ET3” is a human/porcine chimeric coagulation factor VIII molecule.

FIGS. 19 through 20 demonstrate the performance of the disclosed lentiviral vector expressing GFP as compared to an existing commercial system expressing GFP. The results show that we can obtain equivalent results with the novel lentiviral vector system disclosed.

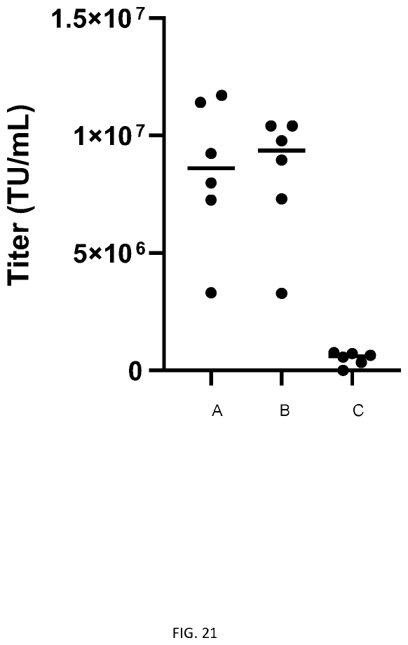

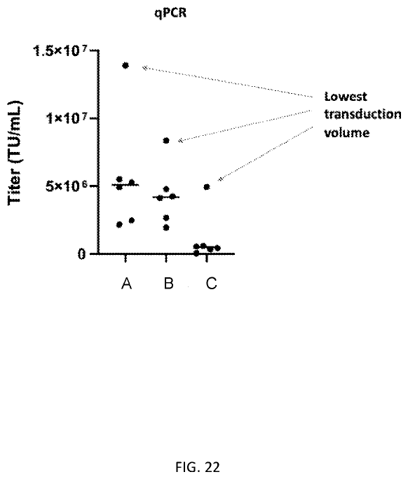

FIGS. 21 and 22 demonstrate that the optimization process met with months to years of lack of success before stumbling upon a combination that worked. FIG. 21 is a comparison, via Flow titer, of early lentiviral vector systems employing known techniques for vector design. In FIG. 21 we demonstrate early results of trials aimed at creating a new lentiviral system which introduced novel modifications to improve safety of the system. This evidence demonstrates that applying conventional methods resulted in poor performance relative to existing commercial systems. Because of these results, we diverged from obvious methods and explored unique methods to solve the problem. The following provides the elements used and correlates to the data in FIG. 21.

FIG. 21, A designates a first commercially available lentiviral system, FIG. 21, B designates a second commercially available system, and FIG. 21, C designates our earlier attempt systems using known vector design techniques. In FIG. 22, is a comparison, via qPCR, of early lentiviral vector systems employing known techniques for vector design compared to various commercial systems. FIG. 22, A designates a first commercially available lentiviral system, FIG. 22, B designates a second commercially available system, and FIG. 22, C designates our earlier attempt systems using known vector design techniques.

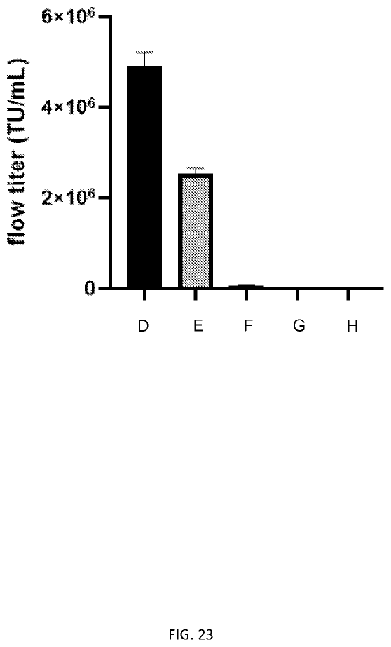

Turning to FIG. 23, the data was derived from an experiment where the transgene plasmid and the GAG/POL plasmid were maintained constant. This data demonstrates the surprising result when the CMV promoter was chosen because it is commonly used to drive expression. For example, early development employed the conventional knowledge of using the CMV promoter to drive all of the plasmids of vector systems. The surprising result was that the CMV promoter resulted in extremely low to no titer as shown in these figures. Only when the conventional knowledge was abandoned did we receive positive results. In FIG. 23, D is the flow titer of a first commercially available VSVG and Rev (our “goal” titer we are trying to match). We will refer to that commercially available system used as “Commercial System D.”) In FIG. 23, E is the flow titer of one of our experimental VSVG and REV (our baseline we were trying to improve on). In FIG. 23, F is the titer achieved when we adopted the CMV promoter into our early experimental system, using our VSVG with CMV and adopting REV from the Commercial System D. This severely reduced titer. In G, we used the REV from Commercial System D with our early experimental VSVG driven by the CMV promoter. This significantly decreased titer. The expected result was that substituting the CMV promoter would improve titer of the base system shown at E. In FIG. 23, H we used the CMV promoter to drive our experimental REV and VSVG, and again, the titer was decreased. The expected result was that adding CMV promoter would increase titer due to an additive effect.

For example, the CMV promoter is used to drive expression. We instead chose a promoter that is not known to have the same robust expression and yet surprisingly our titers increased to match other conventional systems while reducing overall safety profiles. FIG. 23 further demonstrates this point.

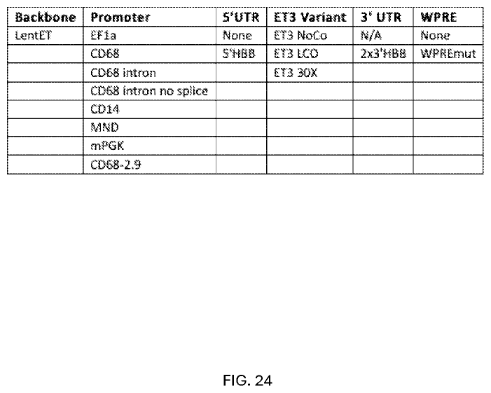

Turning to FIG. 24, we provide a chart demonstrating the elements that would be selected under a disclosed lentiviral system, which creates 388 exemplary constructs. Each line presents a construct. The columns represent the elements of the construct. For example, the firm column provides the identifier for the particular construct disclosed herein. The second column provides the Backbone for the construct. There are multiple possible Backbones, each designated herein by an identifier and disclosed in the Sequence Listings. Each construct can have at least one of the following backbones disclosed herein: LentET (SEQ ID NO: 22); LentET transfer cassette backbone (SEQ ID NO: 44); LentET (SEQ ID NO: 63). LentET transfer cassette backbone of SEQ ID NO: 44 includes an arbitrary spacer sequence “NNNN” to represent the arbitrary transgene.

Each plasmid construct includes at least one of the following promoters, designated in column C of the spreadsheet, Ef1a (SEQ ID NO: 45); CD68 (SEQ ID NO: 46); CD 68 intron promoter (SEQ ID NO: 47); CD 68 intron no splice promoter (SEQ ID NO: 48); MND promoter (SEQ ID NO: 49); CD14 promoter (SEQ ID NO: 50); mPGK promoter (SEQ ID NO: 51); or CD68-2.9 promoter (SEQ ID NO: 52). We have discussed the fact that the transfer plasmid is ambivalent, however, in the context of fVIII therapies, we provide the following fVIII constructs: fVIII (ET3) NoCo (SEQ ID NO: 53), fVIII (ET3) LCO (SEQ ID NO: 54), fVIII (ET3) MCO (SEQ ID NO: 55), fVIII (ET3) 30× (SEQ ID NO: 56). Each construct can have at least one of the following 5′ UTR: None, 5′ HBB (SEQ ID NO: 57). Each construct can have at least one of the following 3′ UTR: None, 3′ HBB (SEQ ID NO: 58), 2×HBB (examples in combination), or 2×3′ HBB (SEQ ID NO: 59). Each construct can have at least one of the following: None, WPREmut (SEQ ID NO: 60). Other constructs are also possible as demonstrated by sequences disclosed herein and plasmid maps.

FIG. 24 demonstrates constructs specifically designed for the expression of the high expression porcine/human FVIII molecule ET3, but we recognize they could be useful for expressing any transgene within the system in lieu of the “ET3 variant” cDNA. In total there are over 384 different LentET ET3 fVIII designs disclosed herein. Rather than list all 96 complete transgene plasmid sequences, we have provided a chart at FIG. 24 that shows every useful combination of the elements we have reduced to practice and give the sequences of the elements in the specification below as SEQ ID NOS: 44 through 60.

The table below provides an exemplary summary of the same. Essentially there are 4 modular parts (promoter, 5′ UTR, ET3 variant, 3′ UTR, and +/−WPRE). Each have a varying number of each element to choose from, giving a large number of potential combinations. The modular parts are illustrated below and shown in FIG. 24:

| | | # of |
|---|---|---|
| Part | Variations | Options |
| Backbone | LentET | 1 |
| Promoter | EF1a, CD68, CD68 intron, CD68 intro | 8 |
| | no splice, CD14, MND, mPGK, CD68-2.9 | |
| FVIII | ET3 NoCo, ET3 LCO, ET3 MCO, ET3 30x | 4 |
| 5′ UTR | None, 5′ HBB | 2 |
| 3′ UTR | None, 3′ HBB, 2x HBB | 3 |
| WPRE | None, WPREmut | 2 |
| | Possible Assemblies | 384+ |

A specific application of these sequences is for the therapeutic use of ex-vivo lentiviral modified stem cells for the treatment of hemophilia. The following table gives a parameterized analysis of the different ET3 fVIII constructs we are claiming and how they differ from each other:

| | Human | Liver | Myeloid | |
|---|---|---|---|---|
| Optimization | CAI | CAI | CAI | Free Energy |
| ET3 NoCo | 0.77 | 0.74 | 0.71 | −1390.2 kcal/mol |
| ET3 LCO | 0.90 | 0.88 | 0.86 | −1612.07 kcal/mol |
| ET3 MCO | 0.87 | 0.84 | 0.80 | −1618.0 kcal/mol |
| ET3 30x | 0.96 | 0.93 | 0.92 | −1934.8 kcal/mol |
| ET3 NoCo | 0.77 | 0.74 | 0.71 | −1390.2 kcal/mol |

| | | Alternative + | |
|---|---|---|---|
| | | strand | Unique ORFs |
| Optimization | CpG Count | ORFs | (relative to NoCo) |
| ET3 LCO | 129 | 48 | n/a |
| ET3 MCO | 0 | 49 | 49 |
| ET3 MCO | 0 | 47 | 47 |
| ET3 30x | 0 | 45 | 45 |

Some combinations of these elements have been disclosed herein.

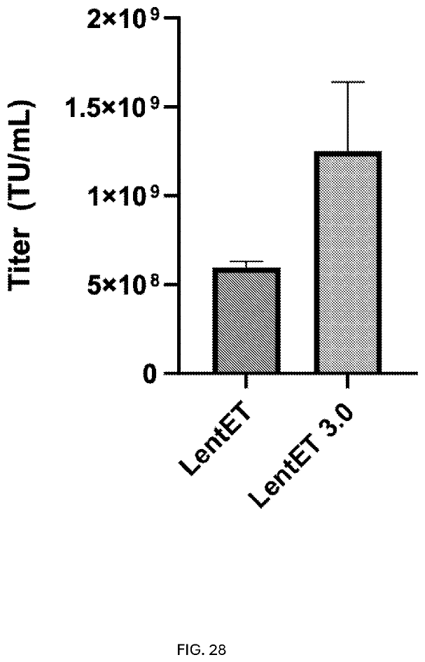

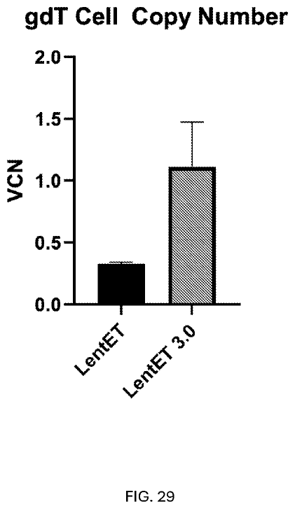

FIG. 28 compares the third generation system disclosed herein, designated on FIG. 29 as LentET 3.0, (a lentiviral system comprising the transfer vector of SEQ ID NO: 63, the GagPol vector of SEQ ID NO: 43, the Env vector of SEQ ID NO: 42; and the Rev vector of SEQ ID NO: 41) with the earlier generation system disclosed herein, designated on FIG. 28 as LentET (a lentiviral system comprising the transfer vector of SEQ ID NO: 22 or SEQ ID NO: 63, the GagPol vector plasmid of SEQ ID NO: 5, the Rev Plasmid of SEQ ID NO: 15, and the Env Plasmid of SEQ ID NO: 19). This FIG. 28 demonstrates that there is increased titer with the further optimized version of the system. LentET 3.0 produces higher titer vector than the original LentET. Vector was produced using the original LentET vector system and the revised LentET 3.0 system. LentET 3.0 achieved vector titers 2-fold higher than those of the original LentET system when titered on 293T cells.

FIG. 29 compares compares the third generation system disclosed herein, designated on FIG. 29 as LentET 3.0, (a lentiviral system comprising the transfer vector of SEQ ID NO: 63, the GagPol vector of SEQ ID NO: 43, the Env vector of SEQ ID NO: 42; and the Rev vector of SEQ ID NO: 41) with the earlier generation system disclosed herein, designated on FIG. 29 as LentET (a lentiviral system comprising the transfer vector of SEQ ID NO: 22 or SEQ ID NO: 63, the GagPol vector plasmid of SEQ ID NO: 5, the Rev Plasmid of SEQ ID NO: 15, and the Env Plasmid of SEQ ID NO: 19). This FIG. 29 demonstrates that there is increased gdT cell copy number in the further optimized system referenced herein as LentET 3.0. LentET 3.0 transduced gdT cell more efficiently than the original LentET. Gamma delta T-cells were transduced at equal multiplicities of infection (MOI) using either LentET or LentET 3.0 GFP vectors. Vector copy number was determined by qPCR. LentET 3.0 resulted in VCNs over 2-fold greater than those of the original LentET system after controlling for MOI.

FIG. 31 is a plasmid map of the LentET 3.0 CMV Gagpol plasmid showing the choice and arrangement of the elements. The data comparison for which is disclosed on FIGS. 28 and 29.

FIG. 32 is a plasmid map of LetET 3.0 VSVG, discussed in more detail above and the data for which is shown at FIGS. 28 and 29.

FIG. 33 is a plasmid map of LetET UbC rev, discussed in more detail above and the data for which is shown at FIGS. 28 and 29.

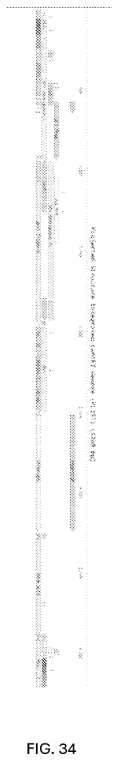

FIG. 34 is a projected map correspondending to SEQ ID NO: 39 and SEQ ID NO: 61. Overall, the LentET 3.0 (a lentiviral system comprising the transfer vector of SEQ ID NO: 63, the GagPol vector of SEQ ID NO: 43, the Env vector of SEQ ID NO: 42; and the Rev vector of SEQ ID NO: 41), has several advantages over earlier lentiviral systems.

LentET Innovations disclosed herein throughout include the following:

### Low Homology Plasmids

The LentET system is a four plasmid system. The LentET 3.0 one 3rd generation lentivector system designed with low homology between the transfer and accessory plasmids. The kanamycin resistance gene has been uniquely codon optimized in each plasmid to create a novel, non-homologous resistance gene sequence in each plasmid. Each plasmid (transfer, VSVG, rev, and gag/pol) also utilize a unique polyA sequences to further reduce homology between plasmids. VSVG and rev further use unique mammalian promoter/intron combinations (PGK/PDK and UbC/SV40, respectively) to further reduce homology between the plasmids. Reduced homology reduces the likelihood of a recombination event leading to the production of replication competent vector. The specific combinations of promoter/introns/polyA signals have been chosen to maximize titer from the system.

### Expression Codon Optimized VSVG and Rev

The rev and VSVG sequences have been Expression Codon Optimized (ECO) to maximize expression in 293T producer cells. Expression of rev and VSVG are often limiting factors in vector titer, and ECO has been shown to improve in vitro protein expression over traditional codon optimization methods. Codon optimization of these genes also reduces homology between the ECO plasmids and wild type HIV in the unlikely event of transfer of these sequences into the finished vector.

### Triple Readthrough Stops

The transfer plasmid contains a unique stop codon sequence flanking the transgene that provides a multi-frame stop codon motive to halt aberrant translational readthrough from either the internal gene promoter contained within the transgene or external promoters proximal to the site of insertion.

### Disrupted HIV Antisense Protein Expression

The start codon of the poorly understood HIV antisense protein (ASP) has been disrupted in the transfer plasmid. The chimeric fusion of the HIV p17 to gp120/gp41 in present in 3rd generation lentiviral systems is anticipated to produce a mutant ASP containing the wildtype N-terminus fused to an un-natural reverse translation of p17, which does not occur in wild-type HIV. The potential for the expression of this product has been removed by elimination of the start codon for ASP present in the antisense reading frame in gp41.

### Mutant WPRE

The woodchuck hepatitis virus posttranscriptional regulatory element (WPRE) is widely used in retroviral gene transfer vectors. However, this element contains an open-reading frame (ORF) encoding a truncated peptide of the woodchuck hepatitis virus X protein (WHX). This reading frame has been disrupted in LentET by incorporating a validated substitution of 6 nucleotides to remove the potential for expression of WHX. (See: Zanta-Boussif, M. A. et al. (2009 May). Validation of a mutated PRE sequence allowing high and sustained transgene expression while abrogating WHV-X protein synthesis: application to the gene therapy of WAS. Gene Therapy, 16 (5). Nature.)

It will be apparent that the precise details of the methods or compositions described may be varied or modified without departing from the spirit of the described embodiments. For example, any of the sequences which are present in the constructs of the present invention can be modified, e.g., to improve transcription, to improve translation, to reduce or alter secondary RNA structure, and/or to decrease recombination. Modifications include, e.g., nucleotide addition, deletion, substitution, and replacements. For example, coding sequences for gag, pol, rev, and tat can be modified by replacing naturally-occurring codons with non-naturally-occurring codons, e.g., to improve translation in a host cell by substituting them with codons which are translated more effectively in the host cell. The host cell can be referred to as a compatible cell, e.g., to indicate the sequence modification has its effect when the sequence is expressed in a particular host cell type. In addition, sequences can be modified to remove regulatory elements, such as the packaging sequence. Sequences can also be altered to eliminate recombination sites. Furthermore, while the disclosed sequences are discussed in the context of the lentiviral vector system, the sequences can be used independently of the lentiviral system and/or in other systems, combinations, or arrangements.

Novel High-Expression fVIII Constructs

Methods and compositions are provided that allow for high-level expression of a factor VIII polypeptide. More specifically, we disclose methods and compositions comprising nucleic acid and amino acid sequences comprising a modified factor VIII that results in high-level expression of the polypeptide. The methods and compositions find use in the treatment of factor VIII deficiency, including, for example, hemophilia A.

In particular, one variation provides an isolated nucleotide sequences comprising an nucleic acid sequence set forth in the sequences NoCo synthetic fVIII (SEQ ID NO: 53), LCO synthetic fVIII (SEQ ID NO: 54), ET3 MCO (SEQ ID NO: 55), ET3-30x (SEQ ID NO: 56); an nucleic acid sequence having at least 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said nucleotide is characterized by high-level expression of the fVIII amino acid sequence, or a fragment thereof as compared to wild type fVIII.

“SQ” refers to the linker region used to replace the FVIII B-domain in B-domain deleted FVIII. The SQ linker sequence is derived human B-domain sequence to retain key glycosylation sites found in the B-domain. ET3 uses a similar, porcine-derived linker sequence designated “OL”. OL also retains key glycosylation sites found in the porcine B-domain and is derived from the porcine B-domain sequence. Importantly, the OL linker sequence is 30 base pairs longer than the SQ linker. This makes substituting SQ into ET3 an attractive approach for AAV therapies, where the limited cargo capacity of AAV favors shorter transgene designs.

In another embodiment of the invention, isolated nucleic acid molecules are provided comprising a nucleotide sequence encoding a polypeptide comprising the amino acid sequence set forth in ET3 NoCo (SEQ ID NO: 53), ET3 LCO (SEQ ID NO: 54), ET3 MCO (SEQ ID NO: 55), ET3-30x (SEQ ID NO: 56); and, a nucleotide sequence having at least 85%, 90%, 95%, 97%, 98%, or 99% sequence identity to the nucleotide sequence encoding a polypeptide 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said nucleotide sequence encodes a polypeptide that is characterized by high-level expression. Expression cassettes, vectors, and cells comprising the nucleic acid molecules of the invention are further provided.

Pharmaceutical compositions comprising the nucleic acid molecules ET3 NoCo (SEQ ID NO: 53), ET3 LCO (SEQ ID NO: 54), ET3 MCO (SEQ ID NO: 55), ET3-30x (SEQ ID NO: 56) and the polypeptides of the invention are also provided.

Methods for the production of a polypeptide are provided. In one embodiment, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein the nucleotide sequence encodes a polypeptide characterized by high-level expression, or a fragment thereof; and, culturing the cell under conditions that allow expression of the nucleotide sequence.

Also provided are methods for increasing the level of expression of the factor VIII polypeptide. In one variation, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56); wherein the nucleotide sequence encodes a polypeptide characterized by high-level expression, or a fragment thereof; and, culturing the cell under conditions that allow expression of the nucleotide sequence.

Also provided is a method for the treatment of factor VIII deficiencies, including, for example, hemophilia A. The method comprises administering to a subject in need thereof a composition comprising a therapeutically effective amount of a polypeptide, where the polypeptide comprises an amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), an amino acid sequence having at least 85%, 90%, 95%, 97%, 98%, or 99% sequence identity to 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said polypeptide is characterized by high-level expression, or a fragment thereof.

Other methods include treating a factor VIII deficiency by administering to a subject in need thereof a composition comprising a therapeutically effective amount of a nucleic acid molecule, where said nucleic acid molecule comprises a nucleotide sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56); a nucleotide sequence encoding a polypeptide comprising the amino acid sequence set forth in ET3 NoCo (SEQ ID NO: 53), ET3 LCO (SEQ ID NO: 54), ET3 MCO (SEQ ID NO: 55), ET3-30x (SEQ ID NO: 56); a nucleotide sequence having at least 85%, 90%, 95%, 97%, 98%, or 99% sequence identity to 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said nucleic acid molecule encodes a polypeptide characterized by high-level expression, or a fragment thereof.

Methods and compositions are provided that allow for high-level expression of a factor VIII polypeptide. More specifically, we disclose methods and compositions comprising nucleic acid and amino acid sequences comprising a modified factor VIII that results in high-level expression of the polypeptide. The methods and compositions find use in the treatment of factor VIII deficiency, including, for example, hemophilia A.

In particular, one variation provides an isolated polypeptide comprising an amino acid sequence set forth in the sequences ET3 NoCo (SEQ ID NO: 53), ET3 LCO (SEQ ID NO: 54), ET3 MCO (SEQ ID NO: 55), ET3-30x (SEQ ID NO: 56); an amino acid sequence having at least 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said polypeptide is characterized by high-level expression, or a fragment thereof.

In another embodiment of the invention, isolated nucleic acid molecules are provided comprising a nucleotide sequence encoding a polypeptide comprising the amino acid sequence set forth in ET3 NoCo (SEQ ID NO: 53), ET3 LCO (SEQ ID NO: 54), ET3 MCO (SEQ ID NO: 55), ET3-30x (SEQ ID NO: 56); and, a nucleotide sequence having at least 85%, 90%, 95%, 97%, 98%, or 99% sequence identity to the nucleotide sequence encoding a polypeptide 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said nucleotide sequence encodes a polypeptide that is characterized by high-level expression. Expression cassettes, vectors, and cells comprising the nucleic acid molecules of the invention are further provided.

Pharmaceutical compositions comprising the nucleic acid molecules and the polypeptides of the invention are also provided.

Methods for the production of a polypeptide are provided. In one embodiment, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein the nucleotide sequence encodes a polypeptide characterized by high-level expression, or a fragment thereof; and, culturing the cell under conditions that allow expression of the nucleotide sequence.

Also provided are methods for increasing the level of expression of the factor VIII polypeptide. In one variation, the method comprises introducing into a cell a nucleic acid molecule comprising a nucleotide sequence encoding a polypeptide comprising the amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56); wherein the nucleotide sequence encodes a polypeptide characterized by high-level expression, or a fragment thereof; and, culturing the cell under conditions that allow expression of the nucleotide sequence.

Also provided is a method for the treatment of factor VIII deficiencies, including, for example, hemophilia A. The method comprises administering to a subject in need thereof a composition comprising a therapeutically effective amount of a polypeptide, where the polypeptide comprises an amino acid sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), an amino acid sequence having at least 85%, 90%, 95%, 97%, 98%, or 99% sequence identity to 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said polypeptide is characterized by high-level expression, or a fragment thereof.

Other methods include treating a factor VIII deficiency by administering to a subject in need thereof a composition comprising a therapeutically effective amount of a nucleic acid molecule, where said nucleic acid molecule comprises a nucleotide sequence set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56); a nucleotide sequence encoding a polypeptide comprising the amino acid sequence set forth in ET3 NoCo (SEQ ID NO: 53), ET3 LCO (SEQ ID NO: 54), ET3 MCO (SEQ ID NO: 55), ET3-30x (SEQ ID NO: 56); a nucleotide sequence having at least 85%, 90%, 95%, 97%, 98%, or 99% sequence identity to 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56), wherein said nucleic acid molecule encodes a polypeptide characterized by high-level expression, or a fragment thereof.

As non-limiting examples, we disclose the LentET transfer vectors with each of these fVIII transgenes. This LentET transfer vector can be used with either LentET or the LentET 3.0 variations of the system or other variations discussed herein. SEQ ID NO: 28 is the LentET transfer plasmid carring the ET3 MCO (SEQ ID NO: 55).

### DETAILED DESCRIPTION

### Overview

We provide methods and compositions that allow for high-level expression of the factor VIII polypeptide. We further provide more humanized versions of factor VIII, which retain the high levels of expression achieved by the factor VIII variants HSQ (SEQ ID NO: 28) and ET3 (SEQ ID NO: 14). The factor VIII polypeptide contains homology-defined proteins domains having the following nomenclature: A1-A2-B-ap-A3-C1-C2. We have identified regions within the domains of a non-human factor VIII polypeptide that promote high-level expression of the factor VIII polypeptide. More particularly, regions of the porcine factor VIII polypeptide that comprises the A1 and ap-A3 regions, and variants and fragments thereof, have been identified which impart high-level expression to both the porcine and human factor VIII polypeptide. We thus provide methods and compositions that use the non-human factor VIII polypeptide sequences which impart high-level expression, and active variants or fragments of these sequences, to construct novel nucleic acid and polypeptide sequences encoding a modified factor VIII polypeptide that results in high-level expression of the encoded factor VIII polypeptide. The modified factor VIII polypeptides characterized by high-level expression are referred to herein as “factor VIII_{SEP}” (Super Expression).

By “high-level expression” is intended the production of a polypeptide at increased levels when compared to the expression levels of the corresponding human factor VIII polypeptide (represented by HSQ, SEQ ID NO: 28) expressed under the same conditions. An increase in polypeptide levels (i.e., high-level expression) comprises at least about 0.5, 1, 2, 3, 4, 5, 6, 7, 8, 9, 10, 11, 12, 13, 14, 15, 20 fold or greater expression of the factor VIII_{SEP} polypeptide compared to the expression levels of the corresponding human factor VIII polypeptide. Alternatively, “high-level expression” can comprise an increase in polypeptide expression levels of at least 1-25 fold, 1-5 fold, 5-10 fold, 10-15 fold, 15-20 fold, 20-25 fold or greater expression levels of the factor VIII_{SEP} when compared to the corresponding human factor VIII polypeptide (represented by HSQ, SEQ ID NO: 28). Methods for assaying “high-level expression” are routine in the art and are outlined in more detail below.

By “corresponding” factor VIII polypeptide is intended a factor VIII polypeptide that comprises an equivalent amino acid sequence. For instance, when a modified factor VIII polypeptide comprising the A1-A2-ap-A3-C1-C2 domains is tested for high-level expression, a human or porcine factor VIII polypeptide containing corresponding domains will be used (i.e., A1-A2-ap-A3-C1-C2). When a fragment of a modified factor VIII polypeptide is tested for high-level expression (i.e., A1-A2-ap-A3), a human or porcine factor VIII polypeptide having the corresponding domains will be tested (i.e., A1-A2-ap-A3).

### Compositions

Compositions of the invention include the nucleic acid molecules encoding factor VIII polypeptides characterized by high-level expression. As outlined in further detail below, the A1 domain of porcine factor VIII (amino acid residues 20-391 of SEQ ID NO:2) and the ap-A3 domain of porcine factor VIII (amino acids 1450-1820 of SEQ ID NO:2) allow for high-level expression of factor VIII. The present invention thus provides methods and compositions comprising factor VIII_{SEP} polypeptides and active variant and active fragments of factor VIII_{SEP} polypeptides characterized by high-level expression.

In particular, the present invention provides for isolated nucleic acid molecules comprising nucleotide sequences encoding the amino acid sequence shown in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56) and active fragments or active variants thereof. Also provided are isolated nucleic acid molecules comprising nucleotide sequences that code for 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56) and active fragments or active variants thereof. Further provided are polypeptides having an amino acid sequence encoded by a nucleic acid molecule described herein, for example, those set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56) and active fragments and active variants thereof.

We disclose isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed factor VIII_{SEP} nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the polypeptides set forth in 85%, 90%, 95%, 97%, 98%, or 99% sequence identity to ET3 NoCo (SEQ ID NO: 53), ET3 LCO (SEQ ID NO: 54), ET3 MCO (SEQ ID NO: 55), ET3-30x (SEQ ID NO: 56) and hence are characterized by high-level expression of the factor VIII polypeptide. Thus, fragments of a nucleotide sequence may range from at least about 10, about 20 nucleotides, about 50 nucleotides, about 100 nucleotides, about 500 nucleotides, about 1000 nucleotides, about 2000 nucleotides, about 3000 nucleotides, about 4000 nucleotides, about 5000 nucleotides, about 6000 nucleotides, about 7000 nucleotides, about 8000 nucleotides, and up to the full-length nucleotide sequence encoding the factor VIII polypeptide of the invention about 9000 nucleotides.

A fragment of a nucleotide sequence of the present invention that encodes a biologically active portion of a factor VIII_{SEP} protein of the invention will encode at least 12, 25, 30, 50, 100, 150, 200, 300, 400, 500, 600, 700, 800, 900, 1000, 1100, 1200, 1300, 1400, 1500, 1600, 1700, 1800, 1900, 2000, 2100, 2200, 2300 contiguous amino acids, or up to the total number of amino acids present in a full-length factor VIII protein of the invention (for example, approximately 1400 to approximately 1600 amino acids for ET3 NoCo (SEQ ID NO: 53), ET3 LCO (SEQ ID NO: 54), ET3 MCO (SEQ ID NO: 55), ET3-30x (SEQ ID NO: 56) and will allow high-level expression of the factor VIII polypeptide.

By “variant” is intended substantially similar sequences. For nucleotide sequences, conservative variants include those sequences that, because of the degeneracy of the genetic code, encode the amino acid sequence of one of the polypeptides of the invention. Variant nucleotide sequences include synthetically derived nucleotide sequences, such as those generated, for example, by using site-directed mutagenesis but which still encode a factor VIII_{SEP} protein characterized by high-level expression. Generally, variants of a particular nucleotide sequence of the invention will have at least at least 75%, 80%, 85%, 86%, 87%, 88%, 89%, preferably about 90%, 91%, 92%, 93%, 94%, 95%, 96%, 97%, and more preferably about 98%, 99%, or more sequence identity to that particular nucleotide sequence as determined by sequence alignment programs described elsewhere herein.

By “variant” protein is intended a protein derived from the polypeptide of ET3 NoCo (SEQ ID NO: 53), ET3 LCO (SEQ ID NO: 54), ET3 MCO (SEQ ID NO: 55), ET3-30x (SEQ ID NO: 56) by deletion (so-called truncation) or addition of one or more amino acids to the N-terminal and/or C-terminal end of the protein; deletion or addition of one or more amino acids at one or more sites in the protein; or substitution of one or more amino acids at one or more sites in the protein. Variant proteins encompassed by the present invention are biologically active, that is they continue to possess the desired biological activity of ET3 NoCo (SEQ ID NO: 53), ET3 LCO (SEQ ID NO: 54), ET3 MCO (SEQ ID NO: 55), ET3-30x (SEQ ID NO: 56), hence they will continue to allow for the high-level expression of the factor VIII polypeptide. Such variants may result from, for example, genetic polymorphism or from human manipulation. Biologically active variants of a polypeptide of the invention will have at least 75%, 80%, 85%, 86%, 87%, 88%, 89%, preferably about 90%, 91%, 92%, 93%, 94%, 95%, 96%, 97%, 98%, 99% or more sequence identity to the amino acid sequence for ET3 NoCo (SEQ ID NO: 53), ET3 LCO (SEQ ID NO: 54), ET3 MCO (SEQ ID NO: 55), ET3-30x (SEQ ID NO: 56) as determined by sequence alignment programs described elsewhere herein using default parameters. A biologically active variant of a protein of the invention may differ from that protein by as few as 1-100, 1-50, 1-25, 1-15 amino acid residues, as few as 1-10, such as 6-10, as few as 5, as few as 4, 3, 2, or even 1 amino acid residue.

Biological activity of the high-level expression factor VIII polypeptides can be assayed by any method known in the art. As discussed above, the high-level expression factor VIII polypeptides are characterized by high-level expression. Assays to measure high-level expression are known in the art. For example, the level of expression of the high-level expression factor VIII polypeptide can be measured by Western blot analysis or ELISA. Other methods include, for example, labeling cell lines expressing the factor VIII polypeptide with ³⁵S-ethionine, followed by immunoprecipitation of radiolabeled factor VIII molecules. Alternatively, the level of expression of the high-level expression factor VIII polypeptide can be assayed for by measuring the activity of the factor VIII polypeptide. For example, increased factor VIII expression could be assayed by measuring factor VIII activity using standard assays known in the art, including a one-stage coagulation assay or a two-stage activity assay. See, for example, U.S. Pat. No. 6,458,561 and the Experimental section below.

Briefly, coagulation assays are based on the ability of factor VIII to shorten the clotting time of plasma derived from a patient with hemophilia A. For example, in the one-stage assay, 0.1 ml hemophilia A plasma (George King Biomedical, Inc.) is incubated with 0.1 ml activated partial thromboplastin reagent (APTT) (Organon Teknika) and 0.01 ml sample or standard, consisting of diluted, citrated normal human plasma, for 5 min at 37° C. in a water bath. Incubation is followed by addition of 0.1 ml 20 mM CaCl₂), and the time for development of a fibrin clot is determined by visual inspection. A unit of factor VIII is defined as the amount present in 1 ml of citrated normal human plasma.

The one-stage assay relies on endogenous activation of factor VIII by activators formed in the hemophilia A plasma, whereas the two-stage assay measures the procoagulant activity of preactivated factor VIII. In the two-stage assay, samples containing factor VIII that are reacted with thrombin are added to a mixture of activated partial thromboplastin and human hemophilia A plasma that is preincubated for 5 min at 37° C. The resulting clotting times are converted to units/ml, based on the same human standard curve described above. See, for example, U.S. Pat. No. 6,376,463.

The high-level expression factor VIII polypeptide may be altered in various ways including amino acid substitutions, deletions, truncations, and insertions. Methods for such manipulations are generally known in the art. For example, amino acid sequence variants of the high-level expression factor VIII polypeptide can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity (i.e., high-level expression) of the high-level expression factor VIII polypeptide may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred. Alternatively, methods to minimize the number of porcine amino acids in the A₁ and ap-A₃ domains of high-level expression factor VIII polypeptide and still continue to retain the high-level expression of the high-level expression factor VIII polypeptide are known in the art and include, for example, established site-directed mutagenesis such as by splicing overlap extension as described elsewhere herein. Obviously, the mutations that will be made in the DNA encoding the variant must not place the sequence out of reading frame and preferably will not create complementary regions that could produce secondary mRNA structure. See, EP Patent Application Publication No. 75,444.

When it is difficult to predict the exact effect of the substitution, deletion, or insertion in advance of doing so, one skilled in the art will appreciate that the effect will be evaluated by routine screening assays. That is, the activity can be evaluated by high-level expression of the factor VIII polypeptide as discussed in detail elsewhere herein.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5: Suppl. 3 (National Biomedical Research Foundation, Washington, D.C.). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences is accomplished using a mathematical algorithm. Specifically, for the purpose of the present invention percent identity of an amino acid sequence is determined using the Smith-Waterman homology search algorithm using an affine 6 gap search with a gap open penalty of 12 5 and a gap extension penalty of 2, BLOSUM matrix 62. The Smith-Waterman homology search algorithm is taught in Smith and Waterman (1981) Adv. Appl. Math 2:482-489, herein incorporated by reference. Alternatively, for the purposes of the present invention percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

It is further recognized that when considering percentage of amino acid identity, some amino acid positions may differ as a result of conservative amino acid substitutions, which do not effect the properties of polynucleotide function. In these instances, percent sequence identity may be adjusted upwards to account for the similarity in conservatively substituted amino acids. Such adjustments are well known in the art. See, for example, Meyers et al. (1988) Computer Applic. Bioi. Sci. 4:11-17.

It is recognized that the variant high-level expression factor VIII polypeptide or fragments thereof can be made (1) by substitution of isolated, plasma-derived animal subunits or human subunits (heavy or light chains) for corresponding human subunits or animal subunits; (2) by substitution of human domains or animal domains (A1, A2, A3, B, C1, and C2) for corresponding animal domains or human domains; (3) by substitution of parts of human domains or animal domains for parts of animal domains or human domains; (4) by substitution of at least one specific sequence including one or more unique human or animal amino acid(s) for the corresponding animal or human amino acid(s); or (5) by substitution of amino acid sequence that has no known sequence identity to factor VIII for at least one sequence including one or more specific amino acid residue(s) in human, animal, or variant factor VIII or fragments thereof. Individual amino acid replacements can be obtain by site-directed mutagenesis of the corresponding segment coding DNA.

In a factor VIII molecule, a “domain”, as used herein, is a continuous sequence of amino acids that is defined by internal amino acid sequence identity and sites of proteolytic cleavage by thrombin. Unless otherwise specified, factor VIII domains include the following amino acid residues, when the sequences are aligned with the human amino acid sequence: A1, residues Alal-Arg372; A2, residues Ser373-Arg740; B, residues Ser741-Arg1648; A3, residues Ser1690-Ile2032; C1, residues Arg2033-Asn2172; C2, residues Ser2173-Tyr2332. The A3-C1-C2 sequence includes residues Ser1690-Tyr2332. The remaining sequence, residues Glu1649Arg1689, is usually referred to as the factor VIII light chain activation peptide. Factor VIII is proteolytically activated by thrombin or factor Xa, which dissociates it from von Willebrand factor, forming factor VIII, which has procoagulant function. The biological function of factor VIIIa is to increase the catalytic efficiency of factor 1 Xa toward factor X activation by several orders of magnitude. Thrombin-activated factor VIIIa is a 160 kDa A1/A2/A3-C1-C2 heterotrimer that forms a complex with factor IXa and factor X on the surface of platelets or monocytes. A “partial domain” as used herein is a continuous sequence of amino acids forming part of a domain. “Subunits” of human or animal (i.e., mouse, pig, dog etc.) factor VIII, as used herein, are the heavy and light chains of the protein. The heavy chain of factor VIII contains three domains, A1, A2, and B. The light chain of factor VIII also contains three domains, A3, C1, and C2. A “unique” amino acid residue or sequence, as used herein, refers to an amino acid sequence or residue in the factor VIII molecule of one species that is different from the homologous residue or sequence in the factor VIII molecule of another species. As used herein, “mammalian factor VIII” includes factor VIII with amino acid sequence derived from any non-human mammal, unless otherwise specified. “Animal”, as used herein, refers to pig and other non-human mammals.

Since current information indicates that the B domain has no inhibitory epitope and has no known effect on factor VIII function, high-level expression factor VIII polypeptide variants of the present invention may have a B domain or a portion thereof. In addition, high-level expression factor VIII polypeptide variants may also have the factor VIII B-domain with the B-domain from porcine or human factor V. See, for example, U.S. Pat. No. 5,004,803. A “B-domainless” variant factor high-level expression factor VIII polypeptide or fragment thereof, as used herein, refers to any one of the variant high-level expression factor VIII polypeptide constructs described herein that lacks the B domain, or a portion thereof.

One of skill in the art will be aware of techniques that allow individual subunits, domains, or continuous parts of domains of animal or human factor VIII cDNA to be cloned and substituted for the corresponding human or porcine subunits, domains, or parts of domains by established mutagenesis techniques and thereby generate a factor VIII_{SEP} or variant or fragment thereof. For example, Lubin et al. (1994) J Biol. Chem. 269 (12): 8639-8641 describes techniques for substituting the porcine A2 domain for the human domain using convenient restriction sites. Other methods for substituting a region of the factor VIII cDNA of one species for the factor VIII cDNA of another species include splicing by overlap extension (“SOE”), as described by Horton et al. (1993) Meth. Enzymol 217:270-279.

### DNA Constructs and Vectors

The nucleotide sequence encoding the high-level expression factor VIII polypeptide polypeptides or active variants or fragments thereof can be contained in a DNA construct. The DNA construct can include a variety of enhancers/promoters from both viral and mammalian sources that drive expression of the high-level expression factor VIII polypeptide polypeptide in the desired cell type. The DNA construct can further contain 3′ regulatory sequences and nucleic acid sequences that facilitate subcloning and recovery of the DNA.

The transcriptional promoter and, if desired, the transcriptional enhancer element are operably linked to the nucleic acid sequence of the factor VIII polypeptide. A “promoter” is defined as a minimal DNA sequence that is sufficient to direct transcription of a nucleic acid sequence. A “transcriptional enhancer element” refers to a regulatory DNA sequence that stimulates the transcription of the adjacent gene. The nucleic acid sequence encoding the factor VIII polypeptide is operably linked to the promoter sequence. See, for example, Goeddel (1990) Gene Expression Technology: Methods in Enzymology 185 (Academic Press, San Diego, CA).

By “operably linked” is intended a functional linkage between the regulatory promoter and the nucleic acid sequence encoding the factor VIII polypeptide. The functional linkage permits gene expression of factor VIII when the appropriate transcription activator proteins are present.

Thus, the DNA construct can include a promoter that may be native or foreign. By “foreign” it is meant a sequence not found in the native organism. Furthermore, the transcription regulatory elements may be heterologous to the nucleotide sequence encoding factor VIII. By “heterologous” is intended any nucleotide sequence not naturally found upstream of the sequence encoding the factor VIII polypeptide. The promoter may be a natural sequence or a synthetic sequence. In addition, the promoter may be constitutively active, tissue-specific, or inducible. A tissue-specific promoter is preferentially activated in a given tissue and results in expression of a gene product in the tissue where activated.

For use in mammalian cells, the promoters may be derived from a virus. For example, commonly used promoters are derived from polyoma, Simian Virus 40 (SV40) and Adenovirus 2. The early and late promoters of SV40 virus are useful as is the major late promoter of adenovirus. Further, it is also possible, and often desirable, to utilize promoter or control sequences normally associated with the desired gene sequence, provided such control sequences are compatible with the host cell system.

In certain variations, the introduction of the nucleotide sequence encoding factor VIII into a cell can be identified in vitro or in vivo by including a marker in the DNA construct. The marker will result in an identifiable change in the genetically transformed cell. Drug selection markers include for example neomycin, puromycin, hygromycin, DHFR, GPT, zeocin and histidinol. Alternatively, enzymes such as herpes simplex virus thymidine kinase (TK) or immunological markers can be used. Further examples of selectable markers are well known in the art.

It is recognized that multiple alterations can be envisioned for the design of the DNA construct used in the methods of the present invention. For instance, the construct may be designed for the insertion of the nucleotide sequence encoding the factor VIII_{SEP} polypeptide using homologous or site-specific recombination systems (i.e., ere or FLP recombination systems).

The DNA construct may also contain at least one additional gene to be co-introduced into the host cells.

The nucleotide sequences can be contained in an expression vector. An “expression vector” is a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art.

Other vectors, including both plasmid and viral vectors, may be used to express a recombinant gene construct in eukaryotic cells depending on the preference and judgment of the skilled practitioner (see, for example, Sambrook et al., Chapter 16). For example, many viral vectors are known in the art including, for example, lentivirus, retroviruses, adeno-associated viruses, and adenoviruses. Other viruses useful for introduction of a gene into a cell include, but a not limited to, herpes virus, mumps virus, poliovirus, Sindbis virus, and vaccinia virus, such as, canary pox virus. The methods for producing replication-deficient viral particles and for manipulating the viral genomes are well known. See, for examples, Rosenfeld et al. (1991) Science 252:431-434, Rosenfeld et al. (1992) Cell 68:143-155, and U.S. Pat. No. 5,882,877 (adenovirus); U.S. Pat. No. 5,139,941 (adeno-associated virus); U.S. Pat. Nos. 4,861,719, 5,681,746, and Miller et al. (1993) Methods in Enzymology 217:581 (retrovirus), all of which are herein incorporated by reference. Therefore, given the knowledge in the art, viral vectors can be readily constructed for use in the introduction of the factor VIII sequences into a cell. Other vectors and expression systems, including bacterial, yeast, and insect cell systems, can be used but are not preferred due to differences in, or lack of, glycosylation.

Factor VIII polypeptides can be expressed in a variety of cells commonly used for culture and recombinant mammalian protein expression. In particular, a number of rodent cell lines have been found to be especially useful hosts for expression of large proteins. Preferred cell lines, available from the American Type Culture Collection, Rockville, Md., include, but are not limited to, baby hamster kidney cells, and chinese hamster ovary (CHO) cells which are cultured using routine procedures and media. Additional cells of interest can include vertebrate cells such as VERO, HeLa cells, W138, COS-7, and MDCK cell lines. For other suitable expression systems see chapters 16 and 17 of Sambrook et al. (1989) Molecular cloning: A Laboratory Manual (2d ed., Cold Spring Harbor Laboratory Press, Plainview, NY). See, Goeddel (1990) in Gene Expression Technology: Methods in Enzymology 185 (Academic Press, San Diego, CA).

### Methods of Expression and Isolation

The DNA construct may be introduced into a cell (prokaryotic or eukaryotic) by standard methods. As used herein, the terms “transformation” and “transfection” are intended to refer to a variety of art recognized techniques to introduce a DNA into a host cell. Such methods include, for example, transfection, including, but not limited to, liposome-polybrene, DEAE dextranmediated transfection, electroporation, calcium phosphate precipitation, microinjection, or velocity driven microprojectiles (“biolistics”). Such techniques are well known by one skilled in the art. See, Sambrook et al. (1989) Molecular Cloning: A Laboratory Manaual (2 ed. Cold Spring Harbor Lab Press, Plainview, NY). Alternatively, one could use a system that delivers the DNA construct in a gene delivery vehicle. The gene delivery vehicle may be viral or chemical. Various viral gene delivery vehicles can be used with the present invention. In general, viral vectors are composed of viral particles derived from naturally occurring viruses. The naturally occurring virus has been genetically modified to be replication defective and does not generate additional infectious viruses. The viral vector also contains a DNA construct capable of expressing the factor VIII protein.

The DNA construct containing nucleic acid sequences encoding the factor VIII_{SEP} polypeptide may also be administered to cell by a non-viral gene delivery vehicle. Such chemical gene delivery vehicles include, for example, a DNA- or RNA-liposome complex formulation or a naked DNA. See, for example, Wang et al. (1987) Proc. Natl. Acad. Sci. U.S.A. 84:7851, U.S. Pat. Nos. 5,844,107, 5,108,921, and Wagner et al. (1991) Proc. Natl. Acad. Sci. U.S.A. 88:4255-4259, all of which are herein incorporated by reference.

It is recognized that the method of introducing the high-level expression factor VIII polypeptide or variant or fragment thereof into a cell can result in either stable integration into the cell genome or transient, episomal expression.

As defined herein, the “expression product” of a DNA encoding a high-level expression factor VIII polypeptide or a fragment or variant thereof is the product obtained from expression of the referenced DNA in a suitable host cell, including such features of pre- or post-translational modification of protein encoded by the referenced DNA, including but not limited to glycosylation, proteolytic cleavage and the like. It is known in the art that such modifications can occur and can differ somewhat depending upon host cell type and other factors, and can result in molecular isoforms of the product, with retention of procoagulant activity. See, for example, Lind et al, (1995) Eur. J. Biochem. 232:1927 incorporated herein by reference.

In a one variation, cDNA encoding high-level expression factor VIII polypeptide or a variant or fragment thereof, is inserted in a mammalian expression vector, such as ReNeo.

Preliminary characterization of the high-level expression factor VIII polypeptide is accomplished by transient expression in the ReNeo expression vector containing the high-level expression factor VIII polypeptide construct in COS-7 cells. A determination of whether active high-level expression factor VIII polypeptide is expressed can then be made. The expression vector construct is used further to stably transfect cells in culture, such as baby hamster kidney cells, using methods that are routine in the art, such as liposome-mediated transfection (Lipofectin™, Life Technologies, Inc.). Expression of the high-level expression factor VIII polypeptide can be confirmed, for example, by sequencing, Northern and Western blotting, or polymerase chain reaction (PCR).

High-level expression factor VIII polypeptide or fragments or variants thereof in the culture media in which the transfected cells stably expressing the protein are maintained can be precipitated, pelleted, washed, and resuspended in an appropriate buffer, and the high-level expression factor VIII polypeptide or variant or fragment thereof is purified by standard techniques, including immunoaffinity chromatography using, for example, monoclonal anti-A2-Sepharose™.

A “fusion protein” or “fusion factor VIII or fragment thereof”, as used herein, is the product of a hybrid gene in which the coding sequence for one protein is extensively altered, for example, by fusing part of it to the coding sequence for a second protein from a different gene to produce a hybrid gene that encodes the fusion protein.

In a further embodiment, the factor VIII or variant or fragment thereof is expressed as a fusion protein from a recombinant molecule in which sequence encoding a protein or peptide that enhances, for example, stability, secretion, detection, isolation, or the like is inserted in place adjacent to the factor VIII encoding sequence. See, for example, U.S. Pat. No. 4,965,199 which discloses a recombinant DNA method for producing factor VIII in mammalian host cells and purification of human factor VIII. Human factor VIII expression on CRG (Chinese hamster ovary) cells and BHKC (baby hamster kidney cells) has been reported. Established protocols for use of homologous or heterologous species expression control sequences including, for example, promoters, operators, and regulators, in the preparation of fusion proteins are known and routinely used in the art. See, Ausubel et al. Current Protocols in Molecular Biology, Wiley Interscience, N.Y, herein incorporated by reference. It is further noted that expression is enhanced by including portions of the B-domain. In particular, the inclusion of those parts of the B domain designated “SQ” (Lind et al. (1995) Eur. J. Biochem. 232:1927, herein incorporated herein by reference) results in favorable expression. “SQ” constructs lack all of the human B domain except for 5 amino acids of the B domain N-terminus and 9 amino acids of the B domain C-terminus.

It is further recognized that the factor VIII polypeptide or variant or fragment thereof may be prepared via reconstitution methods. In this variation factor VIII, variants or fragments thereof are made by isolation of subunits, domains, or continuous parts of domains of plasma-derived factor VIII, followed by reconstitution and purification to produce a factor VIII polypeptide of the invention. Alternatively, the factor VIII, variant or fragment thereof can be made by recombinant DNA methods, followed by reconstitution and purification.

More particularly, the method of preparing a factor VIII by reconstitution methods can be performed via a modification of procedures reported by Fay et al. (1990) J. Biol. Chem. 265:6197; and Lollar et al. (1988) J. Biol. Chem. 263:10451, which involves the isolation of subunits (heavy and light chains) of human and animal factor VIII, followed by recombination of human heavy chain and animal light chain or by recombination of human light chain and animal heavy chain.

Isolation of both human and animal individual subunits involves dissociation of the light chain/heavy chain dimer. This is accomplished, for example, by chelation of calcium with ethylenediaminetetraacetic acid (EDTA), followed by monoS™ HPLC (Pharmacia-LKB, Piscataway, N.J.). Hybrid human/animal factor VIII molecules are reconstituted from isolated subunits in the presence of calcium. Hybrid human light chain/animal heavy chain or animal light chain/human heavy chain factor VIII is isolated from unreacted heavy chains by monoS™ HPLC by procedures for the isolation of porcine factor VIII, such as described by Lollar et al. (1988) Blood 71:137-143 and in U.S. Pat. No. 6,376,463, both of which is herein incorporated by reference.

### Diagnostic Assays

As used herein, “diagnostic assays” include assays that in some manner utilize the antigen-antibody interaction to detect and/or quantify the amount of a particular antibody that is present in a test sample to assist in the selection of medical therapies. There are many such assays known to those of skill in the art. As used herein, however, the factor VIII DNA or variant or fragment thereof and protein expressed therefrom, in whole or in part, can be substituted for the corresponding reagents in the otherwise known assays, whereby the modified assays may be used to detect and/or quantify antibodies to factor VIII. It is the use of these reagents, the factor VIII DNA or variants or fragments thereof or protein expressed therefrom, that permits modification of known assays for detection of antibodies to human or animal factor VIII or to hybrid human/animal factor VIII. As used herein, the factor VIII or variants or fragment thereof that includes at least one epitope of the protein can be used as the diagnostic reagent.

The DNA or amino acid sequence of the factor VIII_{SEP} or variant or fragment thereof can be used in assays as diagnostic reagents for the detection of inhibitory antibodies to human or animal factor VIII, including, for example, samples of serum and body fluids of human patients with factor VIII deficiency. These antibody assays include assays such as ELISA assays, immunoblots, radioimmunoassays, immunodiffusion assays, and assay of factor VIII biological activity (e.g., by coagulation assay). Examples of other assays in which the factor VIII_{SEP} or variant or fragment thereof can be used include the Bethesda assay and anticoagulation assays.

Techniques for preparing these reagents and methods for use thereof are known to those skilled in the art. For example, an immunoassay for detection of inhibitory antibodies in a patient serum sample can include reacting the test sample with a sufficient amount of the factor VIII that contains at least one antigenic site, wherein the amount is sufficient to form a detectable complex with the inhibitory antibodies in the sample.

Nucleic acid and amino acid probes can be prepared based on the sequence of the factor VIII DNA or protein molecule or fragments or variants thereof. In some variations, these can be labeled using dyes or enzymatic, fluorescent, chemiluminescent, or radioactive labels that are commercially available. The amino acid probes can be used, for example, to screen sera or other body fluids where the presence of inhibitors to human, animal, or hybrid human/animal factor VIII is suspected. Levels of inhibitors can be quantitated in patients and compared to healthy controls, and can be used, for example, to determine whether a patient with a factor VIII deficiency can be treated with a factor VIII or active fragment or variant thereof. The cDNA probes can be used, for example, for research purposes in screening DNA libraries.

### Pharmaceutical Compositions

We further provide pharmaceutical compositions comprising the nucleic acid molecules and the polypeptides encoding the high-level expression factor VIII or variants and fragments thereof. Such compositions can comprise nucleic acids and polypeptides of the invention either alone or in combination with appropriate pharmaceutical stabilization compounds, delivery vehicles, and/or carrier vehicles, are prepared according to known methods, as described in Martin et al. Remington's Pharmaceutical Sciences, herein incorporated by reference.

In one variation, the carriers or delivery vehicles for intravenous infusion are physiological saline or phosphate buffered saline.

In another embodiment, suitable stabilization compounds, delivery vehicles, and carrier vehicles include but are not limited to other human or animal proteins such as albumin.

Phospholipid vesicles or liposomal suspensions may also be used as
pharmaceutically acceptable carriers or delivery vehicles. These can be prepared according to methods known to those skilled in the art and can contain, for example, phosphatidylserine-phosphatidylcholine or other compositions of phospholipids or detergents that together impart a negative charge to the surface, since factor VIII binds to negatively charged phospholipid membranes. Liposomes may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the factor VIII_{SEP} of the present invention is then introduced into the container. The container is then swirled by hand to free lipid material from the sides of the container and to disperse lipid aggregates, thereby forming the liposomal suspension.

The factor VIII molecules can be combined with other suitable stabilization compounds, delivery vehicles, and/or carrier vehicles, including vitamin K dependent clotting factors, tissue factor, and von Willebrand factor (vWf) or a fragment of vWf that contains the factor VIII binding site, and polysaccharides such as sucrose.

Factor VIII molecules can also be delivered by gene therapy using delivery means such as retroviral vectors. This method consists of incorporation of a nucleotide sequence encoding desired factor VIII polypeptide into human cells that are transplanted directly into a factor VIII deficient patient or that are placed in an implantable device, permeable to the factor VIII molecules but impermeable to cells, that is then transplanted.

In one variation, the method will be retroviral-mediated gene transfer. In this method, a nucleotide sequence encoding a factor VIII polypeptide is cloned into the genome of a modified retrovirus. The gene is inserted into the genome of the host cell by viral machinery where it will be expressed by the cell. The retroviral vector is modified so that it will not produce virus, preventing viral infection of the host. The general principles for this type of therapy are known to those skilled in the art and have been reviewed in the literature (Kohn et al. (1989) Transfusion 29:812-820).

The factor VIII polypeptide can be stored bound to vWf to increase the half-life and shelf-life of the polypeptide molecule. Additionally, lyophilization of factor VIII can improve the yields of active molecules in the presence of vWf. Current methods for storage of human and animal factor VIII used by commercial suppliers can be employed for storage of recombinant factor VIII. These methods include: (1) lyophilization of factor VIII in a partially-purified state (as a factor VIII “concentrate” that is infused without further purification); (2) immunoaffinity-purification of factor VIII by the Zimmerman method and lyophilization in the presence of albumin, which stabilizes the factor VIII; (3) lyophilization of recombinant factor VIII in the presence of albumin.

Additionally, the factor VIII polypeptides can be stored at 4° C. in 0.6 M NaCl, mM MES, and 5 mM CaCl₂) at pH 6.0. The polypeptides can also be stored frozen in these buffers and thawed with minimal loss of activity.

### Methods of Treatment

Factor VIII or fragments and variant thereof can be used to treat uncontrolled bleeding due to factor VIII deficiency (e.g., intraarticular, intracranial, or gastrointestinal hemorrhage) in hemophiliacs with and without inhibitory antibodies and in patients with acquired factor VIII deficiency due to the development of inhibitory antibodies. The active materials are preferably administered intravenously.

“Factor VIII deficiency,” as used herein, includes deficiency in clotting activity caused by production of defective factor VIII, by inadequate or no production of factor VIII, or by partial or total inhibition of factor VIII by inhibitors. Hemophilia A is a type of factor VIII deficiency resulting from a defect in an X-linked gene and the absence or deficiency of the factor VIII protein it encodes.

Additionally, factor VIII or fragments and variant thereof can be administered by transplantation of cells genetically engineered to produce the factor VIII_{SEP} or by implantation of a device containing such cells, as described above.

In one variation, pharmaceutical compositions of factor VIII or fragments and variants thereof alone or in combination with stabilizers, delivery vehicles, and/or carriers are infused into patients intravenously according to the same procedure that is used for infusion of factor VIII.

The treatment dosages of the factor VIII composition or variants or fragments thereof that must be administered to a patient in need of such treatment will vary depending on the severity of the factor VIII deficiency. Generally, dosage level is adjusted in frequency, duration, and units in keeping with the severity and duration of each patient's bleeding episode. Accordingly, the factor VIII or variants or fragments thereof is included in the pharmaceutically acceptable carrier, delivery vehicle, or stabilizer in an amount sufficient to deliver to a patient a therapeutically effective amount of the hybrid to stop bleeding, as measured by standard clotting assays.

“Specific activity” as used herein, refers to the activity that will correct the coagulation defect of human factor VIII deficient plasma. Specific activity is measured in units of clotting activity per milligram total factor VIII protein in a standard assay in which the clotting time of human factor VIII deficient plasma is compared to that of normal human plasma. One unit of factor VIII activity is the activity present in one milliliter of normal human plasma. In the assay, the shorter the time for clot formation, the greater the activity of the factor VIII being assayed. The specific activity of the factor VIII polypeptides, variant or fragments thereof, may be less than, equal to, or greater than that of either plasma-derived or recombinant human factor VIII.

Factor VIII is classically defined as that substance present in normal blood plasma that corrects the clotting defect in plasma derived from individuals with hemophilia A. The coagulant activity in vitro of purified and partially-purified forms of factor VIII_{SEP} is used to calculate the dose of factor VIII for infusions in human patients and is a reliable indicator of activity recovered from patient plasma and of correction of the in vivo bleeding defect. There are no reported discrepancies between standard assay of novel factor VIII molecules in vitro and their behavior in the dog infusion model or in human patients, according to Lusher et al. New Engl. J. Med. 328:453-459; Pittman et al. (1992) Blood 79:389-397; and Brinkhous et al. (1985) Proc. Natl. Acad. Sci. 82:8752-8755.

The increase of factor VIII in the plasma will be sufficient to produce a therapeutic effect. A “therapeutic effect” is defined as an increase in the blood coagulation activity in the plasma of patients that is greater than the coagulation activity observed in the subject before administration of the factor VIII_{SEP} molecule. In a standard blood clotting assay, the shorter time for clot formation, the greater the activity of factor VIII being assayed. An increase in factor VIII activity in the factor VIII deficient plasma of at least 1% or higher will be therapeutically beneficial.

Usually, the desired plasma factor VIII level to be achieved in the patient through administration of the factor VIII or variant or fragment thereof is in the range of 30-100% of normal. In a one mode of administration of the factor VIII_{SEP} or fragment or variant thereof, the composition is given intravenously at a preferred dosage in the range from about 5 to 50 units/kg body weight, more preferably in a range of 10-50 units/kg body weight, and most preferably at a dosage of 20-40 units/kg body weight; the interval frequency is in the range from about 8 to 24 hours (in severely affected hemophiliacs); and the duration of treatment in days is in the range from 1 to 10 days or until the bleeding episode is resolved. See, for example, Roberts et al. (1990) Hematology, Williams et al. ed. Ch. 153, 1453-1474, herein incorporated by reference. Patients with inhibitors may require more factor VIII_{SEP} or variants or fragments thereof, or patients may require less factor VIII_{SEP} or fragments or variants thereof. As in treatment with human or porcine factor VIII, the amount of factor VIII_{SEP} or fragments or variants infused is defamed by the one-stage factor VIII coagulation assay and, in selected instances, in vivo recovery is determined by measuring the factor VIII in the patient's plasma after infusion. It is to be understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

Treatment can take the form of a single intravenous administration of the composition or periodic or continuous administration over an extended period of time, as required. Alternatively, factor VIII or fragments or variants thereof can be administered subcutaneously or orally with liposomes in one or several doses at varying intervals of time.

Factor VIII or fragments or variants thereof can also be used to treat uncontrolled bleeding due to factor VIII deficiency in hemophiliacs who have developed antibodies to human factor VIII.

### EXPERIMENTAL

### Example 1

### Sequence Characterization of Factor VIII

Both porcine and human factor VIII are isolated from plasma as a two subunit protein. The subunits, known as the heavy chain and light chain, are held together by a non-covalent bond that requires calcium or other divalent metal ions. The heavy chain of factor VIII contains three domains, A1, A2, and B, which are linked covalently. The light chain of factor VIII also contains three domains, designated A3, C1, and C2. The B domain has no known biological function and can be removed, or partially removed from the molecule proteolytically or by recombinant DNA technology methods without significant alteration in any measurable parameter of factor VIII. Human recombinant factor VIII has a similar structure and function to plasma-derived factor VIII, though it is not glycosylated unless expressed in mammalian cells. Both human and porcine activated factor VIII (“factor VIIIa”) have three subunits due to cleavage of the heavy chain between the A1 and A2 domains. This structure is designated A1/A2/A3-C1-C2.

The cDNA sequence of porcine factor VIII corresponding the signal peptide coding region, the A1, B, light chain activity peptide region A3, C1, and C2 domains.

There are eight conserved N-linked glycosylation sites: one in the A1 domain, one in the A2 domain, four in the B domain, one in the A3 domain, and one in the C1 domain. The 19 A and C domain cysteines are conserved, whereas there is divergence of B domain cysteines. Six of the seven disulfide linkages in factor VIII are found at homologous sites in factor V and Ceruloplasmin, and both C domain disulfide linkages are found in factor V (McMullen et al. (1995) Protein Sci. 4:740-746). Human factor VIII contains sulfated tyrosines at positions 346, 718, 719, 723, 1664, and 1680 (Pittman et al. (1992) Biochemistry 31:3315-3325; Michnick et al. (1994) J. Biol. Chem. 269:20095-20102). These residues are conserved in mouse factor VIII and porcine factor VIII although the CLUSTALW program failed to align the mouse tyrosine corresponding to Tyr346 in human factor VIII.

### Example 2

### SUMMARY

Human factor VIII expression levels are significantly lower than levels of other coagulation proteins in vivo and in heterologous expression systems in vitro. Low-level expression of recombinant human factor VIII has constrained the treatment of hemophilia A using recombinant protein infusion and gene therapy protocols. However, recombinant B-domain-deleted porcine factor VIII is expressed at levels 10-14 fold greater than recombinant B-domain-deleted human factor VIII in vitro. To identify sequences of porcine factor VIII necessary for this property, B-domain-deleted human/porcine hybrid factor VIII cDNAs were produced that contained substitution of human sequences with the corresponding porcine sequences. These cDNAs were transiently transfected into COS-7 cells or stably transfected into BHK-derived cells and factor VIII expression into the extracellular media was measured by one-stage coagulation assay. Human/porcine hybrid factor VIII cDNAs containing 1) the A1, A2 and A3 domains of porcine factor VIII and the C1 and C2 domains of human factor VIII, or 2) the A1 and A3 domains of porcine factor VIII and the A2, C1, and C2 domains of human factor VIII demonstrated factor VIII expression levels comparable to porcine factor VIII. A human/porcine hybrid factor VIII molecule demonstrating high-level expression may be valuable for improving factor VIII production for intravenous infusion or for somatic cell gene therapy of hemophilia A.

### Materials

Dulbecco's phosphate-buffered saline, fetal bovine serum (FBS), penicillin, streptomycin, DMEM: F12, serum-free AIM V culture media, Lipofectin, Lipofectamine 2000 and geneticin were purchased from Invitrogen. Baby hamster kidney-derived cells, designated BHK-M cells (Funk et al. (1990) Biochemistry 29:1654-1660), were a gift from Dr. Ross Macgillivray, University of British Columbia. Transient transfections were controlled for transfection efficiency using the RL-CMV vector and Dual-Luciferase Assay Kit (Promega, Madison, WI). Citrated factor VIII-deficient plasma and pooled citrated normal human plasma (FACT) were purchased from George King Biomedical (Overland Park, KA). Activated partial thromboplastin reagent (aPTT) was purchased from Organon Teknika (Durham, NC). Oligonucleotide primers were synthesized by Life Technologies. Pfu DNA polymerase and E. coli XL-1 Blue cells were purchased from Stratagene (La Jolla, CA).

### Construction of Factor VIII Expression Vectors

All of the factor VIII expression vectors in this study were contained in the ReNeo mammalian expression plasmid (Lind et al. (1995) Eur. J. Biochem. 232:1927). The factor VIII cDNA inserts lack endogenous factor VIII 5′-UTR sequence and contain the first 749 of the 1805 nt human factor VIII 3′-UTR.

A human B domain-deleted factor VIII cDNA designed HSQ (SEQ ID NO: 28) was created by cloning the human factor VIII cDNA into the mammalian expression vector ReNeo as described previously (Doering et al. (2002) J. Biol. Chem. 277:38345-38349). The HSQ cDNA encodes an SFSQNPPVLKRHQR (SEQ ID NO:9) linker sequence between the A2 and ap domains. This linker includes the R H Q R (SEQ ID NO:10) recognition sequence for intracellular proteolytic processing by PACE/furin (Seidah et al. (1997) Current Opinion in Biotechnology 8:602-607). This cleavage event converts single chain factor VIII into a heterodimer (Lind et al. (1995) Eur. J. Biochem. 232:19-27). Heterodimeric factor VIII is considered the physiologic form of factor VIII (Fass et al. (1982) Blood 59:594-600).

Various optimized fVIII sequences are provided below.

ET3 NoCo
The high expression human/porcine ET3 FVIII variant that has
not been codon optimized (NoCo)
SEQ ID NO: 53
atgcagctagagctctccacctgtgtctttctgtgtctcttgccactcggctttagtgccatcag
gagatactacctgggcgcagtggaactgtcctgggactaccggcaaagtgaactcctccgtgagc
tgcacgtggacaccagatttcctgctacagcgccaggagctcttccgttgggcccgtcagtcctg
tacaaaaagactgtgttcgtagagttcacggatcaacttttcagcgttgccaggcccaggccacc
atggatgggtctgctgggtcctaccatccaggctgaggtttacgacacggtggtcgttaccctga
agaacatggcttctcatcccgttagtcttcacgctgtcggcgtctccttctggaaatcttccgaa
ggcgctgaatatgaggatcacaccagccaaagggagaaggaagacgataaagtccttcccggtaa
aagccaaacctacgtctggcaggtcctgaaagaaaatggtccaacagcctctgacccaccatgtc
ttacctactcatacctgtctcacgtggacctggtgaaagacctgaattcgggcctcattggagcc
ctgctggtttgtagagaagggagtctgaccagagaaaggacccagaacctgcacgaatttgtact
actttttgctgtctttgatgaagggaaaagttggcactcagcaagaaatgactcctggacacggg
ccatggatcccgcacctgccagggcccagcctgcaatgcacacagtcaatggctatgtcaacagg
tctctgccaggtctgatcggatgtcataagaaatcagtctactggcacgtgattggaatgggcac
cagcccggaagtgcactccatttttcttgaaggccacacgtttctcgtgaggcaccatcgccagg
cttccttggagatctcgccactaactttcctcactgctcagacattcctgatggaccttggccag
ttcctactgttttgtcatatctcttcccaccaccatggtggcatggaggctcacgtcagagtaga
aagctgcgccgaggagccccagctgcggaggaaagctgatgaagaggaagattatgatgacaatt
tgtacgactcggacatggacgtggtccggctcgatggtgacgacgtgtctccctttatccaaatc
cgctcagttgccaagaagcatcctaaaacttgggtacattacattgctgctgaagaggaggactg
ggactatgctcccttagtcctcgcccccgatgacagaagttataaaagtcaatatttgaacaatg
gccctcagcggattggtaggaagtacaaaaaagtccgatttatggcatacacagatgaaaccttt
aagacgcgtgaagctattcagcatgaatcaggaatcttgggacctttactttatggggaagttgg
agacacactgttgattatatttaagaatcaagcaagcagaccatataacatctaccctcacggaa
tcactgatgtccgtcctttgtattcaaggagattaccaaaaggtgtaaaacatttgaaggatttt
ccaattctgccaggagaaatattcaaatataaatggacagtgactgtagaagatgggccaactaa
atcagatccgcggtgcctgacccgctattactctagtttcgttaatatggagagagatctagctt
caggactcattggccctctcctcatctgctacaaagaatctgtagatcaaagaggaaaccagata
atgtcagacaagaggaatgtcatcctgttttctgtatttgatgagaaccgaagctggtacctcac
agagaatatacaacgctttctccccaatccagctggagtgcagcttgaggatccagagttccaag
cctccaacatcatgcacagcatcaatggctatgtttttgatagtttgcagttgtcagtttgtttg
catgaggtggcatactggtacattctaagcattggagcacagactgacttcctttctgtcttctt
ctctggatataccttcaaacacaaaatggtctatgaagacacactcaccctattcccattctcag
gagaaactgtcttcatgtcgatggaaaacccaggtctatggattctggggtgccacaactcagac
tttcggaacagaggcatgaccgccttactgaaggtttctagttgtgacaagaacactggtgatta
ttacgaggacagttatgaagatatttcagcatacttgctgagtaaaaacaatgccattgaaccta
ggagctttgcccagaattcaagaccccctagtgcgagcgctccaaagcctccggtcctgcgacgg
catcagagggacataagccttcctacttttcagccggaggaagacaaaatggactatgatgatat
cttctcaactgaaacgaagggagaagattttgacatttacggtgaggatgaaaatcaggaccctc
gcagctttcagaagagaacccgacactatttcattgctgcggtggagcagctctgggattacggg
atgagcgaatccccccgggcgctaagaaacagggctcagaacggagaggtgcctcggttcaagaa
ggtggtcttccgggaatttgctgacggctccttcacgcagccgtcgtaccgcggggaactcaaca
aacacttggggctcttgggaccctacatcagagcggaagttgaagacaacatcatggtaactttc
aaaaaccaggcgtctcgtccctattccttctactcgagccttatttcttatccggatgatcagga
gcaaggggcagaacctcgacacaacttcgtccagccaaatgaaaccagaacttacttttggaaag
tgcagcatcacatggcacccacagaagacgagtttgactgcaaagcctgggcctacttttctgat
gttgacctggaaaaagatgtgcactcaggcttgatcggcccccttctgatctgccgcgccaacac
cctgaacgctgctcacggtagacaagtgaccgtgcaagaatttgctctgtttttcactatttttg
atgagacaaagagctggtacttcactgaaaatgtggaaaggaactgccgggccccctgccatctg
cagatggaggaccccactctgaaagaaaactatcgcttccatgcaatcaatggctatgtgatgga
tacactccctggcttagtaatggctcagaatcaaaggatccgatggtatctgctcagcatgggca
gcaatgaaaatatccattcgattcattttagcggacacgtgttcagtgtacggaaaaaggaggag
tataaaatggccgtgtacaatctctatccgggtgtctttgagacagtggaaatgctaccgtccaa
agttggaatttggcgaatagaatgcctgattggcgagcacctgcaagctgggatgagcacgactt
tcctggtgtacagcaagaagtgtcagactcccctgggaatggcttctggacacattagagatttt
cagattacagcttcaggacaatatggacagtgggccccaaagctggccagacttcattattccgg
atcaatcaatgcctggagcaccaaggagcccttttcttggatcaaggtggatctgttggcaccaa
tgattattcacggcatcaagacccagggtgcccgtcagaagttctccagcctctacatctctcag
tttatcatcatgtatagtcttgatgggaagaagtggcagacttatcgaggaaattccactggaac
cttaatggtcttctttggcaatgtggattcatctgggataaaacacaatatttttaaccctccaa
ttattgctcgatacatccgtttgcacccaactcattatagcattcgcagcactcttcgcatggag
ttgatgggctgtgatttaaatagttgcagcatgccattgggaatggagagtaaagcaatatcaga
tgcacagattactgcttcatcctactttaccaatatgtttgccacctggtctccttcaaaagctc
gacttcacctccaagggaggagtaatgcctggagacctcaggtgaataatccaaaagagtggctg
caagtggacttccagaagacaatgaaagtcacaggagtaactactcagggagtaaaatctctgct
taccagcatgtatgtgaaggagttcctcatctccagcagtcaagatggccatcagtggactctct
tttttcagaatggcaaagtaaaggtttttcagggaaatcaagactccttcacacctgtggtgaac
tctctagacccaccgttactgactcgctaccttcgaattcacccccagagttgggtgcaccagat
tgccctgaggatggaggttctgggctgcgaggcacaggacctctactga
ET3 LCO
The high expression human/porcine ET3 FVIII variant
that has been codon optimized for high expression
in the liver (LCO)
SEQ ID NO: 54
atgcagctggaactgtctacctgtgtgtttctgtgtctgctgcctctggggttttctgctatcag
gagatactatctgggagctgtggagctgtcctgggactacaggcagtctgagctgctgagagaac
tgcatgtggataccagattcccagctacagctccaggagctctgcctctgggcccatctgtgctg
tacaagaaaacagtctttgtggagtttacagaccagctgttctctgtggccaggccaagaccacc
ttggatgggactgctgggaccaaccatccaggctgaggtgtatgatacagtggtggtgaccctga
aaaacatggcctcccatcctgtgagcctgcatgctgtgggggtgtccttctggaagtcctctgag
ggagctgagtatgaagaccatacctcccagagggagaaagaagatgataaggtgctgcctggcaa
aagccagacctatgtctggcaggtgctgaaggagaatggaccaactgcttctgacccaccatgcc
tgacctactcttatctgtcccatgtggatctggtgaaggacctgaattctggactgattggagct
ctgctggtgtgtagagagggaagcctgaccagagaaagaacccagaacctgcatgagtttgtcct
gctgtttgctgtgtttgatgaagggaagagctggcactctgccaggaatgactcctggaccagag
ctatggatccagctcctgctagagctcagcctgctatgcacacagtcaatggctatgtgaatagg
tctctgccaggactgattggctgccataagaaatctgtctattggcatgtgattggaatgggcac
cagccctgaggtgcattctatcttcctggaaggccacacctttctggtcaggcaccatagacagg
cctctctggagatctcccctctgaccttcctgacagctcagacctttctgatggacctggggcag
ttcctgctgttttgccatatctcttcccaccatcatggaggaatggaggctcatgtcagggtgga
atcctgtgctgaggaaccacagctgagaagaaaggctgatgaggaagaggactatgatgataacc
tgtatgactctgatatggatgtggtgaggctggatggggatgatgtcagccctttcatccagatc
aggtctgtggccaagaaacatccaaagacctgggtccactacattgctgctgaagaggaagattg
ggactatgcccccctggtgctggctcctgatgatagatcctacaaaagccagtatctgaacaatg
ggccccagaggattggaaggaagtacaagaaagtgaggttcatggcctatacagatgagaccttt
aagaccagagaggctatccagcatgaatctgggatcctgggacctctgctgtatggagaagtggg
ggataccctgctgatcatcttcaagaaccaggcctccaggccatacaatatctatccccatggca
tcacagatgtgagaccactgtacagcaggagactgcccaagggggtcaaacacctgaaggatttc
cccatcctgcctggagagatctttaagtataaatggacagtcacagtggaagatgggcctaccaa
gtctgatccaaggtgcctgaccagatactatagctcttttgtgaacatggagagagacctggctt
ctggactgattggacccctgctgatctgttacaaagagtctgtggaccagaggggcaaccagatc
atgtctgataagagaaatgtcatcctgttctctgtgtttgatgagaacaggagctggtacctgac
agagaacatccagaggttcctgccaaatccagctggagtgcagctggaggacccagaatttcagg
cttccaacatcatgcatagcatcaatggctatgtgtttgatagcctgcagctgtctgtctgcctg
catgaggtggcctactggtatatcctgtccattggagctcagacagacttcctgtctgtgttctt
tagtgggtacacctttaagcataaaatggtgtatgaggataccctgaccctgttccccttttctg
gggagacagtgttcatgtccatggaaaaccctggcctgtggatcctggggtgccacaactctgac
ttcaggaatagaggaatgacagccctgctgaaagtgtccagctgtgataagaatacaggggatta
ctatgaggactcttatgaagatatctctgcttatctgctgagcaagaacaatgccattgagccca
ggtcttttgctcagaactccagacctccatctgcttctgctcctaagccacctgtgctgagaaga
catcagagggacatctccctgcctaccttccagccagaggaagataaaatggactatgatgatat
cttcagcacagagaccaagggggaagattttgacatctatggagaggatgaaaaccaggatccaa
gatccttccagaagagaaccagacactactttattgctgctgtggagcagctgtgggactatggg
atgtctgaaagcccaagggccctgaggaacagagctcagaatggagaggtgcccagattcaagaa
agtggtgttcagagagtttgctgatggcagctttacccagccatcttacaggggggagctgaaca
agcatctggggctgctgggaccctatatcagagctgaggtggaagataacatcatggtgaccttc
aagaatcaggcttctaggccctactccttttattcttccctgatctcctaccctgatgatcagga
gcagggagctgaacctaggcacaactttgtgcagccaaatgagaccagaacctacttttggaagg
tgcagcatcacatggctcccacagaggatgaatttgactgcaaagcttgggcctatttttctgat
gtggacctggagaaggatgtgcattctggcctgattgggcctctgctgatctgtagggccaacac
cctgaatgctgctcatggaagacaggtcacagtgcaggagtttgctctgttctttaccatctttg
atgaaaccaagagctggtacttcacagagaatgtggaaaggaattgcagagccccctgtcatctg
cagatggaggaccctaccctgaaggaaaactacaggttccatgccatcaatggatatgtcatgga
taccctgcctggcctggtcatggctcagaaccagaggatcagatggtacctgctgtctatgggat
ccaatgagaatatccatagcatccacttctctggccatgtcttttctgtgaggaagaaagaggaa
tacaaaatggctgtgtacaatctgtatcctggggtctttgagacagtggaaatgctgccaagcaa
agtgggaatctggagaattgagtgcctgattggggaacacctgcaggctgggatgagcaccacct
tcctggtgtactctaagaaatgtcagaccccactggggatggcctctggacatatcagggacttc
cagatcacagcttctggacagtatggacagtgggctccaaagctggctagactgcactattctgg
ctccatcaatgcctggtctaccaaagagccattctcctggatcaaggtggacctgctggccccca
tgatcatccatggaatcaaaacccagggagctaggcagaagttcagctctctgtacatctcccag
tttatcatcatgtatagcctggatgggaagaaatggcagacctacagaggcaattccactgggac
cctgatggtcttctttggaaatgtggattcctctggcatcaagcacaacatcttcaatccaccca
tcattgccaggtacatcaggctgcatcctacccactatagcatcaggtctaccctgagaatggag
ctgatgggatgtgacctgaacagctgttctatgccactgggcatggagtccaaggctatctctga
tgcccagatcacagcttcttcctacttcaccaatatgtttgctacctggtccccaagcaaggcta
gactgcacctgcagggaagatccaatgcttggagaccccaggtgaacaatcctaaggagtggctg
caggtggacttccagaaaaccatgaaggtcacaggggtgaccacccagggagtgaaatctctgct
gacctccatgtatgtcaaggagttcctgatcagctcttcccaggatggccaccagtggaccctgt
tctttcagaatggcaaggtcaaagtgttccaggggaatcaggactcttttaccccagtggtgaac
tccctggatcctccactgctgaccaggtacctgagaatccatcctcagagctgggtgcaccagat
tgctctgagaatggaggtcctgggatgtgaagctcaggacctgtattga
ET3 MCO
The high expression human/porcine ET3 FVIII
variant that has not codon optimized for high
expression in the myeloid lineage cells (MCO)
SEQ ID NO: 55
atgcagctggagctctcaacctgtgtgttcctctgcctgctccccctgggattttcagctatcag
gagatactatctgggagcagtggaactgtcctgggactacaggcagtcagagctgctcagagaac
tgcatgtggatactaggttccctgcaacagctcctggagcactgccactgggaccttcagtgctg
tacaagaaaactgtctttgtggagtttacagaccagctgttcagtgtggccaggcccaggccccc
ctggatggggctgctgggacccaccatccaggctgaagtgtatgatactgtggtggtgaccctga
aaaacatggcctctcatccagtcagcctgcatgctgtgggagtgagcttctggaagagcagtgag
ggagctgagtatgaagaccatacctcacagagggagaaagaagatgataaggtgctgccaggaaa
aagccagacctatgtgtggcaggtgctgaaggagaatggccctacagcttcagatcctccctgcc
tcacatactcttatctgagccatgtggatctggtgaaggacctcaatagtggcctgattggggca
ctgctggtgtgcagagaggggtccctcacaagggaaagaactcagaacctgcatgagtttgtcct
gctctttgctgtgtttgatgagggaaagtcctggcactcagcaaggaatgacagctggaccaggg
ctatggacccagcaccagccagagctcagccagctatgcacactgtcaatggctatgtgaatagg
tccctgcctggactcattggctgccataagaaatcagtctattggcatgtgattggaatgggcac
cagcccagaggtgcattccatcttcctggaaggccacacatttctggtcaggcaccatagacagg
ccagcctggagatcagcccactgactttcctcacagcacagacatttctgatggacctggggcag
ttcctgctcttttgccatatctcaagtcaccatcatggagggatggaggctcatgtcagggtgga
aagctgtgcagaggaacctcagctgaggaggaaggcagatgaggaagaggactatgatgataacc
tgtatgactcagatatggatgtggtgaggctggatggagatgatgtcagcccattcatccagatc
aggtcagtggctaagaaacaccctaagacctgggtccactacattgcagctgaagaggaagattg
ggactatgcacccctggtgctggccccagatgatagaagttacaaatctcagtatctgaacaatg
ggccccagaggattggaaggaagtacaagaaagtgaggttcatggcttatactgatgagaccttt
aagacaagagaggcaatccagcatgaaagtggcatcctgggaccactgctctatggagaagtggg
ggataccctgctcatcatcttcaagaaccaggcctcaaggccttacaatatctatccccatggca
tcacagatgtgaggcctctctacagcaggagactgcccaagggagtcaaacacctcaaggatttc
cccatcctgccaggggaaatcttcaagtataaatggacagtcactgtggaagatgggccaactaa
gtcagatcctaggtgcctgaccaggtactattctagctttgtgaacatggagagggacctggctt
caggactgattggacctctgctcatctgctacaaagaatcagtggaccagaggggcaaccagatc
atgagtgataagagaaatgtcatcctgttctcagtgtttgatgagaataggagttggtatctgac
agaaaacatccagaggttcctgcctaatcctgcaggagtgcagctggaggacccagaatttcagg
cttcaaacatcatgcatagtatcaatggctatgtgtttgatagtctgcagctctctgtctgcctg
catgaggtggcctactggtatatcctcagcattggagctcagactgacttcctgagtgtgttctt
ttcaggctacacattcaagcataagatggtctatgaagataccctgacactcttccccttttctg
gggagactgtgtttatgagcatggaaaacccaggcctgtggattctggggtgccacaacagtgac
ttcaggaatagagggatgactgctctgctcaaagtgtcctcatgtgataagaatactggagatta
ctatgaggactcttatgaagatatcagtgcatatctgctctccaaaaacaatgccattgagccca
ggtcatttgctcagaacagtagaccaccttctgcaagtgcaccaaagcctccagtgctgaggaga
caccagagggacatcagcctgccaaccttccagcctgaggaagataaaatggactatgatgatat
cttctccactgagaccaagggggaagattttgacatctatggagaggatgaaaaccaggacccca
ggtccttccagaagaggaccagacactactttattgcagctgtggagcagctgtgggactatggc
atgtctgaatcacctagagctctgaggaacagagcacagaatggggaggtgcccaggttcaagaa
agtggtgttcagagaatttgcagatggctcttttacccagcctagctacaggggggagctcaaca
agcatctggggctgctgggaccctatatcagagcagaggtggaagataacatcatggtgacattc
aagaatcaggcctcaagaccctacagtttttatagttctctgatcagctacccagatgatcagga
gcagggggctgaaccaaggcacaactttgtgcagcctaatgagacaagaacttacttttggaagg
tccagcatcacatggctcccacagaggatgagtttgactgcaaggcctgggcatatttttctgat
gtggacctggagaaggatgtgcatagtggcctcattgggccactgctcatctgcagggcaaacac
actgaatgctgcacatggcaggcaggtcactgtgcaggagtttgccctgttctttacaatctttg
atgaaactaagtcctggtacttcacagagaatgtggaaaggaattgcagagccccctgccatctc
cagatggaggacccaactctgaaggaaaactacaggttccatgctatcaatggatatgtcatgga
tacactgccaggcctggtgatggcacagaaccagaggatcaggtggtatctgctcagcatggggt
ccaatgagaatatccattctatccacttctcaggacatgtcttttcagtgaggaagaaagaggaa
tataaaatggctgtgtacaatctgtatccaggggtctttgagacagtggaaatgctgcctagcaa
agtggggatctggagaattgagtgcctcattggagaacacctgcaggcagggatgtccaccacat
ttctggtgtactcaaagaaatgccagactcccctggggatggcaagtggacatatcagggacttc
cagatcactgcatcaggacagtatggacagtgggcaccaaagctggctaggctccactatagtgg
ctctatcaatgcttggagtaccaaagagcctttctcttggatcaaggtggatctgctggccccca
tgatcatccatggaatcaaaacacagggagctagacagaagttcagctccctgtacatcagtcag
tttatcatcatgtattctctggatgggaagaaatggcagacctacaggggcaatagcactgggac
actgatggtcttctttggaaatgtggattcaagtggcatcaagcacaacatcttcaatcctccca
tcattgccaggtacatcagactgcatcccacacactattcaatcaggagtactctcagaatggag
ctgatggggtgtgacctcaacagctgctccatgccactgggaatggaatccaaggcaatctcaga
tgcccagatcactgcttctagctacttcaccaatatgtttgcaacatggtcacccagtaaagcaa
ggctgcacctccagggaaggtccaatgcttggagaccccaggtgaacaatccaaaggagtggctg
caggtggactttcagaaaaccatgaaggtcacaggggtgactacccagggagtgaaaagtctgct
cacctctatgtatgtcaaggagttcctgatctcctcaagtcaggatggccaccagtggacactgt
tctttcagaatggcaaggtcaaagtgttccaggggaatcaggacagctttacaccagtggtgaac
agcctggacccccctctgctcactagatatctgagaatccatccacagagctgggtgcaccagat
tgcactcagaatggaggtcctgggctgtgaagcccaggacctgtattga
ET3 30x
The high expression human/porcine ET3 FVIII
variant that has been Expression Codon Optimized (ECO)
30 times using the ECO optimization tool
SEQ ID NO: 56
atgcaactggaactgtcaacatgtgtcttcttgtgcctgctgcccctgggcttctctgccatcag
gaggtactacctgggggctgtggagctgtcctgggactacaggcagtctgagctgctgagggagc
tgcatgtggacaccaggttccctgccacagcccctggggccctgcccctgggcccctctgtgctg
tacaagaagacagtgtttgtggagttcacagaccagctgttctctgtggccaggcccaggccccc
ctggatgggcctgctgggccccaccatccaggctgaggtgtatgacacagtggtggtgaccctga
agaacatggcctcccaccctgtgtccctgcatgctgtgggggtgtccttctggaagtcctctgag
ggggctgagtatgaggaccacaccagccagagggagaaggaggatgacaaggtgctgcctggcaa
gagccagacctatgtgtggcaggtgctgaaggagaatggccccacagcctctgaccccccctgcc
tgacctacagctacctgtcccatgtggacctggtgaaggacctgaactctggcctgattggggcc
ctgctggtgtgcagggaggggtccctgaccagggagaggacccagaacctgcatgagtttgtgct
gctgtttgctgtgtttgatgagggcaagagctggcactctgccaggaatgactcctggaccaggg
ccatggaccctgcccctgccagggcccagcctgccatgcacacagtgaatgggtatgtgaacagg
agcctgcctggcctgattgggtgccacaagaagtctgtgtactggcatgtgattgggatgggcac
ctcccctgaggtgcactccatcttcctggagggccacaccttcctggtgaggcaccacaggcagg
cctccctggagatcagccccctgaccttcctgacagcccagaccttcctgatggacctgggccag
ttcctgctgttctgccacatcagcagccaccaccatgggggcatggaggcccatgtgagggtgga
gtcctgtgctgaggagccccagctgaggaggaaggctgatgaggaggaggactatgatgacaacc
tgtatgactctgacatggatgtggtgaggctggatggggatgatgtgtcccccttcatccagatc
aggtctgtggccaagaagcaccccaagacctgggtgcactacattgctgctgaggaggaggactg
ggactatgcccccctggtgctggcccctgatgacaggagctacaagagccagtacctgaacaatg
gcccccagaggattggcaggaagtacaagaaggtgaggttcatggcctacacagatgagaccttc
aagaccagggaggccatccagcatgagtctggcatcctgggccccctgctgtatggggaggtggg
ggacaccctgctgatcatcttcaagaaccaggcctccaggccctacaacatctacccccatggca
tcacagatgtgaggcccctgtacagcaggaggctgcccaagggggtgaagcacctgaaggacttc
cccatcctgcctggggagatcttcaagtacaagtggacagtgacagtggaggatggccccaccaa
gtctgaccccaggtgcctgaccaggtactacagctcctttgtgaacatggagagggacctggcct
ctggcctgattggccccctgctgatctgctacaaggagtctgtggaccagaggggcaaccagatc
atgtctgacaagaggaatgtgatcctgttctctgtgtttgatgagaacaggagctggtacctgac
agagaacatccagaggttcctgcccaaccctgctggggtgcagctggaggaccctgagttccagg
cctccaacatcatgcactccatcaatgggtatgtgtttgactccctgcagctgtctgtgtgcctg
catgaggtggcctactggtacatcctgtccattggggcccagacagacttcctgtctgtgttctt
ctctggctacaccttcaagcacaagatggtgtatgaggacaccctgaccctgttccccttctctg
gggagacagtgttcatgagcatggagaaccctggcctgtggatcctggggtgccacaactctgac
ttcaggaacaggggcatgacagccctgctgaaggtgtcctcctgtgacaagaacacaggggacta
ctatgaggactcctatgaggacatctctgcctacctgctgtccaagaacaatgccattgagccca
ggtcctttgcccagaacagcaggcccccctctgcctctgcccccaagccccctgtgctgaggagg
caccagagggacatcagcctgcccaccttccagcctgaggaggacaagatggactatgatgacat
cttctccacagagaccaagggggaggactttgacatctatggggaggatgagaaccaggacccca
ggagcttccagaagaggaccaggcactacttcattgctgctgtggagcagctgtgggactatggc
atgtctgagtcccccagggccctgaggaacagggcccagaatggggaggtgcccaggttcaagaa
ggtggtgttcagggagtttgctgatggctccttcacccagccctcctacaggggggagctgaaca
agcacctgggcctgctgggcccctacatcagggctgaggtggaggacaacatcatggtgaccttc
aagaaccaggcctccaggccctacagcttctacagcagcctgatcagctaccctgatgaccagga
gcagggggctgagcccaggcacaactttgtgcagcccaatgagaccaggacctacttctggaagg
tgcagcaccacatggcccccacagaggatgagtttgactgcaaggcctgggcctacttctctgat
gtggacctggagaaggatgtgcactctggcctgattggccccctgctgatctgcagggccaacac
cctgaatgctgcccatgggaggcaggtgacagtgcaggagtttgccctgttcttcaccatctttg
atgagaccaagagctggtacttcacagagaatgtggagaggaactgcagggccccctgccacctg
cagatggaggaccccaccctgaaggagaactacaggttccatgccatcaatgggtatgtgatgga
caccctgcctggcctggtgatggcccagaaccagaggatcaggtggtacctgctgtccatgggct
ccaatgagaacatccactccatccacttctctggccatgtgttctctgtgaggaagaaggaggag
tacaagatggctgtgtacaacctgtaccctggggtgtttgagacagtggagatgctgccctccaa
ggtgggcatctggaggattgagtgcctgattggggagcacctgcaggctggcatgagcaccacct
tcctggtgtacagcaagaagtgccagacccccctggggatggcctctggccacatcagggacttc
cagatcacagcctctggccagtatggccagtgggcccccaagctggccaggctgcactactctgg
ctccatcaatgcctggagcaccaaggagcccttcagctggatcaaggtggacctgctggccccca
tgatcatccatggcatcaagacccagggggccaggcagaagttcagcagcctgtacatcagccag
ttcatcatcatgtacagcctggatggcaagaagtggcagacctacaggggcaactccacaggcac
cctgatggtgttctttgggaatgtggactcctctggcatcaagcacaacatcttcaaccccccca
tcattgccaggtacatcaggctgcaccccacccactacagcatcaggagcaccctgaggatggag
ctgatggggtgtgacctgaacagctgcagcatgcccctggggatggagtccaaggccatctctga
tgcccagatcacagcctccagctacttcaccaatatgtttgccacctggagcccctccaaggcca
ggctgcacctgcagggcaggtccaatgcctggaggccccaggtgaacaaccccaaggagtggctg
caggtggacttccagaagaccatgaaggtgacaggggtgaccacccagggggtgaagagcctgct
gaccagcatgtatgtgaaggagttcctgatcagcagcagccaggatggccaccagtggaccctgt
tcttccagaatgggaaggtgaaggtgttccaggggaaccaggactccttcacccctgtggtgaac
agcctggacccccccctgctgaccaggtacctgaggatccacccccagtcctgggtgcaccagat
tgccctgaggatggaggtgctggggtgtgaggcccaggacctgtactgataatag
The 5′ human beta globin UTR
SEQ ID NO: 57
Actcttctggtccccacagactcagagagaac
3′ HBB. The 3′ human beta globin UTR
SEQ ID NO: 58
gctcgctttcttgctgtccaatttctattaaaggttcctttgttccctaagtccaactactaaac
tgggggatattatCaagggccttgagcatctggattctgcctaataaaaaacatttattttcatt
gcaa
2x3′ HBB Tandem copies of the human beta globin UTR
SEQ ID NO: 59
gctcgctttcttgctgtccaatttctattaaaggttcctttgttccctaagtccaactactaaac
tgggggatattatCaagggccttgagcatctggattctgcctaataaaaaacatttattttcatt
gcaagctcgctttcttgctgtccaatttctattaaaggttcctttgttccctaagtccaactact
aaactgggggatattatCaagggccttgagcatctggattctgcctaataaaaaacatttatttt
cattgcaa
WPREmut
Woodchuck Hepatitis Virus Posttranscriptional
Regulatory Element - a version of WPRE modified
to remove a potential start codon and initiation
of an open reading frame. These modified nucleotides are
given in CAPITAL LETTERS. This element is designed to
enhances expression of transgenes delivered by
retroviral vectors.
SEQ ID NO: 60
atcaacctctggattacaaaatttgtgaaagattgactgatattcttaactatgttgctcctttt
acgctgtgtggatatgctgctttaatgcctctgtatcatgctattgcttcccgtacggctttcgt
tttctcctccttgtataaatcctggttgctgtctctttatgaggagttgtggcccgttgtccgtc
aacgtggcgtggtgtgctctgtgtttgctgacgcaacccccactggctggggcattgccaccacc
tgtcaactcctttctgggactttcgctttccccctcccgatcgccacggcagaactcatcgccgc
ctgccttgcccgctgctggacaggggctaggttgctgggcactgataattccgtggtgttgtcgg
ggaaATCATcgtcctttccTtggctgctcgcctgtgttgccaactggatcctgcgcgggacgtcc
ttctgctacgtcccttcggctctcaatccagcggacctcccttcccgaggccttctgccggttct
gcggcctctcccgcgtcttcgctttcggcctccgacgagtcggatctccctttgggccgcctccc
cgcctg
Structure of Integrated LentET Genome (from PL391)
SEQ ID NO: 61
tggaagggctaattcactcccaaCgaagacaagatctgctttttgcTtgtactgggtctctctgg
ttagaccagatctgagcctgggagctctctggctaactagggaacccactgcttaagcctcaata
aagcttgccttgagtgcttcaagtagtgtgtgcccgtctgttgtgtgactctggtaactagagat
ccctcagacccttttagtcagtgtggaaaatctctagcagggtctctctggttagaccagatctg
agcctgggagctctctggctaactagggaacccactgcttaagcctcaataaagcttgccttgag
tgcttcaagtagtgtgtgcccgtctgttgtgtgactctggtaactagagatccctcagacccttt
tagtcagtgtggaaaatctctagcagtggcgcccgaacagggacCtgaaagcgaaagggaaacca
gaggagctctctcgacgcaggactcggcttgctgaagcgcgcacggcaagaggcgaggggcggcg
actggtgagtacgccaaaaattttgactagcggaggctagaaggagagagatgggtgcgagagcg
tcagtattaagcgggggagaattagatCGcgatgggaaaaaattcggttaaggccagggggaaag
aaaaaatataaattaaaacatatagtatgggcaagcagggagctagaacgattcgcagttaatcc
tggcctgttagaaacatcagaaggctgtagacaaatactgggacagctacaaccatcccttcaga
caggatcagaagaacttagatcattatataatacagtagcaaccctctattgtgtgcatcaaagg
atagagataaaagacaccaaggaagctttagacaagatagaggaagagcaaaacaaaagtaagaC
CaCCgcacagcaagcagcCgctgatcttcagacctggaggaggagatatgagggacaattggaga
agtgaattatataaatataaagtagtaaaaattgaaccattaggagtagcacccaccaaggcaaa
gagaagagtggtgcagagagaaaaaagagcagtgggaataggagctttgttccttgggttcttgg
gagcagcaggaagcactatgggcgcagcctcaatgacgctgacggtacaggccagacaattattg
tctggtatagtgcagcagcagaacaatttgctgagggctattgaggcgcaacagcatctgttgca
actcacagtctggggcaAcaagcagctccaggcaagaatcctggctgtggaaagatacctaaagg
atcaacagctcctggggatttggggttgctctggaaaactcatttgcaccactgctgtgccttgg
aatgctagttggagtaataaatctctggaacagatttggaatcacacgacctggatggagtggga
cagagaaattaacaattacacaagcttaatacactccttaattgaagaatcgcaaaaccagcaag
aaaagaatgaacaagaattattggaattagataaatgggcaagtttgtggaattggtttaacata
acaaattggctgtggtatataaaattattcataatgatagtaggaggcttggtaggtttaagaat
agtttttgctgtactttctatagtgaatagagttaggcagggatattcaccattatcgtttcaga
cccacctcccaaccccgaggggacccgacaggcccgaaggaatagaagaagaaggtggagagaga
gacagagacagatccattcgattagtgaacggatcggcagtattcatccacaattttaaaagaaa
aggggggattggggggtacagtgcaggggaaagaatagtagacataatagcaacagacatacaaa
ctaaagaattacaaaaacaaattacaaaaattcaaaatttttaatcagctacttattaattaggt
gactagttgattaaaaccggtatcGattagtccaatttgttaaagacaggatatcagtggtccag
gctctagttttgactcaacaatatcaccagctgaagcctatagagtacgagccatagatagaata
aaagattttatttagtctccagaaaaaggggggaatgaaagaccccacctgtaggtttggcaagc
taggatcaaggttaggaacagagagacagcagaatatgggccaaacaggatatctgtggtaagca
gttcctgccccggctcagggccaagaacagTtggaacagcagaatatgggccaaacaggatatct
gtggtaagcagttcctgccccggctcagggccaagaacagatggtccccagatgcggtcccgccc
tcagcagtttctagagaaccatcagatgtttccagggtgccccaaggacctgaaatgaccctgtg
ccttatttgaactaaccaatcagttcgcttctcgcttctgttcgcgcgcttctgctccccgagct
caataaaagagcccacaacccctcactcggcgcgctcgaggccgccaccatggtcagcaagggcg
aggaactgttcaccggggtggtgcccatcctggtcgagctggacggcgacgtaaacggccacaag
ttcagcgtgtccggcgagggcgagggcgatgccacctacggcaagctgaccctgaagttcatctg
taccaccggcaagctgcccgtgccctggcccaccctcgtgaccaccctgacctacggcgtgcaat
gcttcagccgctaccccgaccacatgaagcagcacgacttcttcaagtccgccatgcccgaaggc
tacgtccaggagcgcaccatcttcttcaaggacgacggcaactacaagacccgcgccgaggtgaa
gttcgagggcgacaccctggtgaaccgcatcgagctgaagggcatcgacttcaaggaggacggca
acatcctggggcacaagctggagtacaactacaacagccacaacgtctatatcatggccgacaag
cagaagaacggcatcaaggtgaacttcaagatccgccacaacatcgaggacggcagcgtgcaact
cgccgaccactaccagcagaacacccccatcggcgacggccccgtgctgctgcccgacaaccact
acctgagcacccagtccgccctgagcaaagaccccaacgagaagcgcgatcacatggtcctgctg
gagttcgtgaccgccgccgggatcactctcggcatggacgagctgtacaagtaggcggccgccgc
cggtgtagttgattaactcagctacttaataattaggtgaatcaacctctggattacaaaatttg
tgaaagattgactgatattcttaactatgttgctccttttacgctgtgtggatatgctgctttaa
tgcctctgtatcatgctattgcttcccgtacggctttcgttttctcctccttgtataaatcctgg
ttgctgtctctttatgaggagttgtggcccgttgtccgtcaacgtggcgtggtgtgctctgtgtt
tgctgacgcaacccccactggctggggcattgccaccacctgtcaactcctttctgggactttcg
ctttccccctcccgatcgccacggcagaactcatcgccgcctgccttgcccgctgctggacaggg
gctaggttgctgggcactgataattccgtggtgttgtcggggaaATCATcgtcctttccTtggct
gctcgcctgtgttgccaactggatcctgcgcgggacgtccttctgctacgtcccttcggctctca
atccagcggacctcccttcccgaggccttctgccggttctgcggcctctcccgcgtcttcgcttt
cggcctccgacgagtcggatctccctttgggccgcctccccgcctgtaattaggtgactagttga
ttaaatcagctacttatccaatgacttacaaggcagctgtagatcttagccactttttaaaagaa
aaggggggactggaagggctaattcactcccaaCgaagacaagatctgctttttgcTtgtactgg
gtctctctggttagaccagatctgagcctgggagctctctggctaactagggaacccactgctta
agcctcaataaagcttgccttgagtgcttcaagtagtgtgtgcccgtctgttgtgtgactctggt
aactagagatccctcagacccttttagtcagtgtggaaaatctctagca
Exemplary Transfer Plasmid Vector Sequence
SEQ ID NO: 62
CGGTCATTTCTTACCTCCCCTTCCCTCTCCCACCTGCTACTGGGTGCATCTCTGCTCCCCCCTTC
CCCAGCAGATGGTTACCTTTGGGCTGTTGCTTTCTTGTCACCATCTGAGTTCTCAGACGCTGGAA
AGCCATGTTCTCGGCTCTGTGAATGACAATGCTGACTGGAGTGCTGCCCCTCTGTAAAGGGCTGG
GTGTGGATGGTCACAAGCCCCTCACATGCCTCAGCCAAGAGGAAGTAGTACAGGGGTCAGCCCAG
AGGTCCAGGGGAAAGGAGTGGAAACCGATTTCCCCACCAAGGGAGGGGCCTGTACCTCAGCTGTT
CCCATAGCTACTTGCCACAACTGCCAAGCAAGTTTCGCTGAGTTTGACACATGGATCCCTGTGGA
TCAACTGCCCTAGGACTCCGTTTGCACCCATGTGACACTGTTGACTTTGCCCTGATGAAGCAGGG
CCAACAGTCCCCTAACTTAATTACAAAAACTAATGACTAAGAGAGAGGTGGCTAGAGCTGAGGCC
CCTGAGTCAGGCTGTGGGTGGGATCATCTCCAGTACAGGAAGTGAGACTTTCATTTCCTCCTTTC
CAAGAGAGGGCTGAGGGAGCAGGGTTGAGCAACTGGTGCAGACAGCCTAGCTGGACTTTGGGTGA
GGCGGTTCAGCCGCGGCCGCAAAACCTCTCGAGCCACCATGCAGCTAGAGCTCTCCACCTGTGTC
TTTCTGTGTCTCTTGCCACTCGGCTTTAGTGCCATCAGGAGATACTACCTGGGCGCAGTGGAACT
GTCCTGGGACTACCGGCAAAGTGAACTCCTCCGTGAGCTGCACGTGGACACCAGATTTCCTGCTA
CAGCGCCAGGAGCTCTTCCGTTGGGCCCGTCAGTCCTGTACAAAAAGACTGTGTTCGTAGAGTTC
ACGGATCAACTTTTCAGCGTTGCCAGGCCCAGGCCACCATGGATGGGTCTGCTGGGTCCTACCAT
CCAGGCTGAGGTTTACGACACGGTGGTCGTTACCCTGAAGAACATGGCTTCTCATCCCGTTAGTC
TTCACGCTGTCGGCGTCTCCTTCTGGAAATCTTCCGAAGGCGCTGAATATGAGGATCACACCAGC
CAAAGGGAGAAGGAAGACGATAAAGTCCTTCCCGGTAAAAGCCAAACCTACGTCTGGCAGGTCCT
GAAAGAAAATGGTCCAACAGCCTCTGACCCACCATGTCTTACCTACTCATACCTGTCTCACGTGG
ACCTGGTGAAAGACCTGAATTCGGGCCTCATTGGAGCCCTGCTGGTTTGTAGAGAAGGGAGTCTG
ACCAGAGAAAGGACCCAGACCTGCACGAATTTGTACTACTTTTTGCTGTCTTTGATGAAGGGAAA
AGTTGGCACTCAGCAAGAAATGACTCCTGGACACGGGCCATGGATCCCGCACCTGCCAGGGCCCA
GCCTGCAATGCACACAGTCAATGGCTATGTCAACAGGTCTCTGCCAGGTCTGATCGGATGTCATA
AGAAATCAGTCTACTGGCACGTGATTGGAATGGGCACCAGCCCGGAAGTGCACTCCATTTTTCTT
GAAGGCCACACGTTTCTCGTGAGGCACCATCGCCAGGCTTCCTTGGAGATCTCGCCACTAACTTT
CCTCACTGCCCAGACATTCCTGATGGACCTTGGCCAGTTCCTACTGTTTTGTCATATCTCTTCCC
ACCACCATGGTGGCATGGAGGCTCACGTCAGAGTAGAAAGCTGCGCCGAGGAGCCCCAGCTGCGG
AGGAAAGCTGATGAAGAGGAAGATTATGATGACAATTTGTACGACTCGGACATGGACGTGGTCCG
GCTCGATGGTGACGACGTGTCTCCCTTTATCCAAATCCGCTCAGTTGCCAAGAAGCATCCTAAAA
CTTGGGTACATTACATTGCTGCTGAAGAGGAGGACTGGGACTATGCTCCCTTAGTCCTCGCCCCC
GATGACAGAAGTTATAAAAGTCAATATTTGAACAATGGCCCTCAGCGGATTGGTAGGAAGTACAA
AAAAGTCCGATTTATGGCATACACAGATGAAACCTTTAAGACGCGTGAAGCTATTCAGCATGAAT
CAGGAATCTTGGGACCTTTACTTTATGGGGAAGTTGGAGACACACTGTTGATTATATTTAAGAAT
CAAGCAAGCAGACCATATAACATCTACCCTCACGGAATCACTGATGTCCGTCCTTTGTATTCAAG
GAGATTACCAAAAGGTGTAAAACATTTGAAGGATTTTCCAATTCTGCCAGGAGAAATATTCAAAT
ATAAATGGACAGTGACTGTAGAAGATGGGCCAACTAAATCAGATCCTCGGTGCCTGACCCGCTAT
TACTCTAGTTTCGTTAATATGGAGAGAGATCTAGCTTCAGGACTCATTGGCCCCCTCCTCATCTG
CTACAAAGAATCTGTAGATCAAAGAGGAAACCAGATAATGTCAGACAAGAGGAATGTCATCCTGT
TTTCTGTATTTGATGAGAACCGAAGCTGGTACCTCACAGAGAATATACAACGCTTTCTCCCCAAT
CCAGCTGGAGTGCAGCTTGAGGATCCAGAGTTCCAAGCCTCCAACATCATGCACAGCATCAATGG
CTATGTTTTTGATAGTTTGCAGTTGTCAGTTTGTTTGCATGAGGTGGCATACTGGTACATTCTAA
GCATTGGAGCACAGACTGACTTCCTTTCTGTCTTCTTCTCTGGATATACCTTCAAACACAAAATG
GTCTATGAAGACACACTCACCCTATTCCCATTCTCAGGAGAAACTGTCTTCATGTCGATGGAAAA
CCCAGGTCTATGGATTCTGGGGTGCCACAACTCAGACTTTCGGAACAGAGGCATGACCGCCTTAC
TGAAGGTTTCTAGTTGTGACAAGAACACTGGTGATTATTACGAGGACAGTTACGAAGATATTTCA
GCATACTTGCTGAGTAAAAACAATGCCATTGAACCTAGGAGCTTTGCCCAGAATTCAAGACCCCC
TAGTGCGAGCGCTCCAAAGCCTCCGGTCCTGCGACGGCATCAGAGGGACATAAGCCTTCCTACTT
TTCAGCCGGAGGAAGACAAAATGGACTATGATGATATCTTCTCAACTGAAACGAAGGGAGAAGAT
TTTGACATTTACGGTGAGGATGAAAATCAGGACCCTCGCAGCTTTCAGAAGAGAACCCGACACTA
TTTCATTGCTGCGGTGGAGCAGCTCTGGGATTACGGGATGAGCGAATCCCCCCGGGCGCTAAGAA
ACAGGGCTCAGAACGGAGAGGTGCCTCGGTTCAAGAAGGTGGTCTTCCGGGAATTTGCTGACGGC
TCCTTCACGCAGCCGTCGTACCGCGGGGAACTCAACAAACACTTGGGGCTCTTGGGACCCTACAT
CAGAGCGGAAGTTGAAGACAACATCATGGTAACTTTCAAAAACCAGGCGTCTCGTCCCTATTCCT
TCTACTCGAGCCTTATTTCTTATCCGGATGATCAGGAGCAAGGGGCAGAACCTCGACACAACTTC
GTCCAGCCAAATGAAACCAGAACTTACTTTTGGAAAGTGCAGCATCACATGGCACCCACAGAAGA
CGAGTTTGACTGCAAAGCCTGGGCCTACTTTTCTGATGTTGACCTGGAAAAAGATGTGCACTCAG
GCTTGATCGGCCCCCTTCTGATCTGCCGCGCCAACACCCTGAACGCTGCTCACGGTAGACAAGTG
ACCGTGCAAGAATTTGCTCTGTTTTTCACTATTTTTGATGAGACAAAGAGCTGGTACTTCACTGA
AAATGTGGAAAGGAACTGCCGGGCCCCCTGCCATCTGCAGATGGAGGACCCCACTCTGAAAGAAA
ACTATCGCTTCCATGCAATCAATGGCTATGTGATGGATACACTCCCTGGCTTAGTAATGGCTCAG
AATCAAAGGATCCGATGGTATCTGCTCAGCATGGGCAGCAATGAAAATATCCATTCGATTCATTT
TAGCGGACACGTGTTCAGTGTACGGAAAAAGGAGGAGTATAAAATGGCCGTGTACAATCTCTATC
CGGGTGTCTTTGAGACAGTGGAAATGCTACCGTCCAAAGTTGGAATTTGGCGAATAGAATGCCTG
ATTGGCGAGCACCTGCAAGCTGGGATGAGCACGACTTTCCTGGTGTACAGCAAGAAGTGTCAGAC
TCCCCTGGGAATGGCTTCTGGACACATTAGAGATTTTCAGATTACAGCTTCAGGACAATATGGAC
AGTGGGCCCCAAAGCTGGCCAGACTTCATTATTCCGGATCAATCAATGCCTGGAGCACCAAGGAG
CCCTTTTCTTGGATCAAGGTGGATCTGTTGGCACCAATGATTATTCACGGCATCAAGACCCAGGG
TGCCCGTCAGAAGTTCTCCAGCCTCTACATCTCTCAGTTTATCATCATGTATAGTCTTGATGGGA
AGAAGTGGCAGACTTATCGAGGAAATTCCACTGGAACCTTAATGGTCTTCTTTGGCAATGTGGAT
TCATCTGGGATAAAACACAATATTTTTAACCCTCCAATTATTGCTCGATACATCCGTTTGCACCC
AACTCATTATAGCATTCGCAGCACTCTTCGCATGGAGTTGATGGGCTGTGATTTAAATAGTTGCA
GCATGCCATTGGGAATGGAGAGTAAAGCAATATCAGATGCACAGATTACTGCTTCATCCTACTTT
ACCAATATGTTTGCCACCTGGTCTCCTTCAAAAGCTCGACTTCACCTCCAAGGGAGGAGTAATGC
CTGGAGACCTCAGGTGAATAATCCAAAAGAGTGGCTGCAAGTGGACTTCCAGAAGACAATGAAAG
TCACAGGAGTAACTACTCAGGGAGTAAAATCTCTGCTTACCAGCATGTATGTGAAGGAGTTCCTC
ATCTCCAGCAGTCAAGATGGCCATCAGTGGACTCTCTTTTTTCAGAATGGCAAAGTAAAGGTTTT
TCAGGGAAATCAAGACTCCTTCACACCTGTGGTGAACTCTCTAGACCCACCGTTACTGACTCGCT
ACCTTCGAATTCACCCCCAGAGTTGGGTGCACCAGATTGCCCTGAGGATGGAGGTTCTGGGCTGC
GAGGCACAGGACCTCTACTGAGCGCCGGTGAAGGGCGAATTCCAGCACACTGGCGGCCGTTACTA
GTGGATCCGAGCTCGGTACCTTTAAGACCAATGACTTACAAGGCAGCTGTAGATCTTAGCCACTT
TTTAAAAGAAAAGGGGGGACTGGAAGGGCTAATTCACTCCCAACGAAGACAAGATCTGCTTTTTG
CTTGTACTGGGTCTCTCTGGTTAGACCAGATCTGAGCCTGGGAGCTCTCTGGCTAACTAGGGAAC
CCACTGCTTAAGCCTCAATAAAGCTTGCCTTGAGTGCTTCAAGTAGTGTGTGCCCGTCTGTTGTG
TGACTCTGGTAACTAGAGATCCCTCAGACCCTTTTAGTCAGTGTGGAAAATCTCTAGCAGCATCT
AGAGGGCCCGTTTAAACCCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGCGGTGGGCTCTATGGC
TTCTACTGGGCGGTTTTATGGACAGCAAGCGAACCGGAATTGCCAGCTGGGGCGCCCTCTGGTAA
GGTTGGGAAGCCCTGCAAAGTAAACTGGATGGCTTTCTTGCCGCCAAGGATCTGATGGCGCAGGG
GATCAAGCTCTGATCAAGAGACAGGATGAGGATCGTTTCGCATGATTGAACAAGATGGATTGCAC
GCAGGTTCTCCGGCCGCTTGGGTGGAGAGGCTATTCGGCTATGACTGGGCACAACAGACAATCGG
CTGCTCTGATGCCGCCGTGTTCCGGCTGTCAGCGCAGGGGCGCCCGGTTCTTTTTGTCAAGACCG
ACCTGTCCGGTGCCCTGAATGAACTGCAAGACGAGGCAGCGCGGCTATCGTGGCTGGCCACGACG
GGCGTTCCTTGCGCAGCTGTGCTCGACGTTGTCACTGAAGCGGGAAGGGACTGGCTGCTATTGGG
CGAAGTGCCGGGGCAGGATCTCCTGTCATCTCACCTTGCTCCTGCCGAGAAAGTATCCATCATGG
CTGATGCAATGCGGCGGCTGCATACGCTTGATCCGGCTACCTGCCCATTCGACCACCAAGCGAAA
CATCGCATCGAGCGAGCACGTACTCGGATGGAAGCCGGTCTTGTCGATCAGGATGATCTGGACGA
AGAGCATCAGGGGCTCGCGCCAGCCGAACTGTTCGCCAGGCTCAAGGCGAGCATGCCCGACGGCG
AGGATCTCGTCGTGACCCATGGCGATGCCTGCTTGCCGAATATCATGGTGGAAAATGGCCGCTTT
TCTGGATTCATCGACTGTGGCCGGCTGGGTGTGGCGGACCGCTATCAGGACATAGCGTTGGCTAC
CCGTGATATTGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGAATTATTAAC
GCTTACAATTTCCTGATGCGGTATTTTCTCCTTACGCATCTGTGCGGTATTTCACACCGCATCAG
GTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATAGCACGTGCTAAAACTTCA
TTTTTAATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCGCAGATACCAA
ATACTGTTCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAGCTATGAGAA
AGCGCCACGCTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGTTCTTGCTGCT
TCGCGATGTACGGGCCAGATATACGCGTTGACATTGATTATTGACTAGTTATTAATAGTAATCAA
TTACGGGGTCATTAGTTCATAGCCCATATATGGAGTTCCGCGTTACATAACTTACGGTAAATGGC
CCGCCTGGCTGACCGCCCAACGACCCCCGCCCATTGACGTCAATAATGACGTATGTTCCCATAGT
AACGCCAATAGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTATT
AGTCATCGCTATTACCATGGTGATGCGGTTTTGGCAGTACATCAATGGGCGTGGATAGCGGTTTG
ACTCACGGGGATTTCCAAGTCTCCACCCCATTGACGTCAATGGGAGTTTGTTTTGGCACCAAAAT
CAACGGGACTTTCCAAAATGTCGTAACAACTCCGCCCCATTGACGCAAATGGGCGGTAGGCGTGT
ACGGTGGGAGGTCTATATAAGCAGAGCTCGTTTAGTGAACCGGGGTCTCTCTGGTTAGACCAGAT
CTGAGCCTGGGAGCTCTCTGGCTAACTAGGGAACCCACTGCTTAAGCCTCAATAAAGCTTGCCTT
GAGTGCTTCAAGTAGTGTGTGCCCGTCTGTTGTGTGACTCTGGTAACTAGAGATCCCTCAGACCC
TTTTAGTCAGTGTGGAAAATCTCTAGCAGTGGCGCCCGAACAGGGACTTGAAAGCGAAAGGGAAA
CCAGAGGAGCTCTCTCGACGCAGGACTCGGCTTGCTGAAGCGCGCACGGCAAGAGGCGAGGGGCG
GCGACTGGTGAGTACGCCAAAAATTTTGACTAGCGGAGGCTAGAAGGAGAGAGATGGGTGCGAGA
GCGTCAGTATTAAGCGGGGGAGAATTAGATCGCGATGGGAAAAAATTCGGTTAAGGCCAGGGGGA
AAGAAAAAATATAAATTAAAACATATAGTATGGGCAAGCAGGGAGCTAGAACGATTCGCAGTTAA
TCCTGGCCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAAAAGTAA
GACCACCGCACAGCAAGCGGCCGCTGATCTTCAGACCTGGAGGAGGAGATATGAGGGACAATTGG
AGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCT
TGGGAGCAGCAGGAAGCACTATGGGCGCAGCGTCAATGACGCTGACGGTACAGGCCAGACAATTA
TTGTCTGGTATAGTGCAGCAGCAGAACAATTTGCTGAGGGCTATTGAGGCGCAACAGCATCTGTT
GCAACTCACAGTCTGGGGCATCAAGCAGCTCCAGGCAAGAATCCTGGCTGTGGAAAGATACCTAA
AGGATCAACAGCTCCTGGGGATTTGGGGTTGCTCTGGAAAACTCATTTGCACCACTGCTGTGCCT
TGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGC
AAGAAAAGAATGAACAAGAATTATTGGAATTAGATAAATGGGCAAGTTTGTGGAATTGGTTTAAC
ATAACAAATTGGCTGTGGTATATAAAATTATTCATAATGATAGTAGGAGGCTTGGTAGGTTTAAG
AATAGTTTTTGCTGTACTTTCTATAGTGAATAGAGTTAGGCAGGGATATTCACCATTATCGGTTA
ACTTTTAAAAGAAAAGGGGGGATTGGGGGGTACAGTGCAGGGGAAAGAATAGTAGACATAATAGC
AACAGACATACAAACTAAAGAATTACAAAAACAAATTACAAAAATTCAAAATTTTATCGATCACG
AGACTAGCCTCGAATCTGCAGAATTCGCCCTTATCGAT

### SEQ ID NO: 62 Plasmid Genetic Features

CD68 Promoter located at base pairs 1-664, fVIII gene located at base pairs 689 through 5092, 3′LTR located at base pairs 5268 through 5725, Kanamycin Resistence Gene located at base pairs 5893 through 6697, pUC Origen of Replication located at base pairs 7012 through 7599, pCMV-5′ LTR located at base pairs 8299 through 8480, Psi Region located at base pairs 8531 through 8668, Splice donor region located at base pairs 8588 through 8591, envelope located at base pairs 8997 through 9871, Central Polypurine Tract (cPPT) located at base pairs 9754 through 9871.

Sequences produced by SOE mutagenesis were confirmed by dideoxy DNA sequencing.

Transient Expression of Factor VIII from COS-7 Cells

COS-7 cells were grown to 70-80% confluence in 2 cm² wells containing 1 ml DMEM: F12 supplemented with 10% FBS, 100 units/ml penicillin and 100 μg/ml streptomycin. Cells were transfected with a 2000:1 mass ratio of factor VIII plasmid:luciferase plasmid DNA using Lipofectamine 2000. Twenty-four hours after transfection the cells were rinsed twice with 1 ml of PBS and 0.5 ml of serum-free AIM V medium was added to each well. Cells were cultured 24 hr before the conditioned media was harvested and factor VIII activity was measured as described below.

Stable Expression of Factor VIII from Baby Hamster Kidney-Derived (BHK-M) Cells

BHK-M cells were transfected using Lipofectin along with an ReNeo plasmid containing factor VIII cDNA and cultured in the presence of DMEM: F12 containing 10% FBS, 100 units/ml penicillin, 100 μg/ml streptomycin and 500 μg/ml geneticin for 10 days. The ReNeo vector contains the neomycin phosphotransferase gene for resistance to the antibiotic geneticin. Twenty-four to 72 geneticin resistant clones were screened for factor VIII production. The clone from each cDNA construct that displayed the highest level of factor VIII activity was transferred into a 75 cm² flask, grown to 90-95% confluence and then switched to 25 ml serum-free AIM V media. After 24 hr, the conditioned media was replaced with 25 ml fresh serum-free media AIM V and cultured for an additional 24 hr. Harvested media from each time point was assayed for factor VIII activity as described below.

### Factor VIII Assay

Factor VIII activity was measured by one-stage coagulation assay using a ST art Coagulation Instrument (Diagnostica Stago, Asnieres, France). Five μl of sample or standard was added to 50 μl of factor VIII-deficient plasma, followed by addition of 50 μl aPTT reagent and incubation for 3 min at 37° C. Fifty microliters of 20 mM CaCl₂ was added to initiate the reaction, and the time required to develop a fibrin clot was measured viscometrically. Standard curves were generated using several dilutions of pooled normal human plasma and subjected to linear regression analysis of the clotting time versus the logarithm of the reciprocal plasma dilution. For determination of factor VIII activity, samples were diluted in HEPES buffered saline to a concentration within the range of the standard curve.

### Results

To identify constructs that exhibit high-level expression, variant factor VIII molecules ET3 NoCo (SEQ ID NO: 53), ET3 LCO (SEQ ID NO: 54), ET3 MCO (SEQ ID NO: 55), ET3-30x (SEQ ID NO: 56) were constructed and their expression levels in COS-7 and BHK-M cells were measured. After COS-7 cell transfection, the expression plasmid is not integrated into genomic DNA, but is present transiently as an episomal DNA. Expression levels from COS-7 cells represent an average of the cell population. There is a significant increase in expression of ET3 NoCo (SEQ ID NO: 53), ET3 LCO (SEQ ID NO: 54), ET3 MCO (SEQ ID NO: 55), ET3-30x (SEQ ID NO: 56) compared to HSQ. In contrast, expression of A1 S7-8 and A1-All were not increased compared to HSQ.

### DISCUSSION

Recombinant B domain-deleted porcine factor VIII is expressed at levels up to 14-fold greater than recombinant human factor VIII (Doering et al. (2002) J. Biol. Chem. 277:38345-38349). The levels are substantially greater than in previously published reports of factor VIII expression (Table II). The mechanism for the high expression phenomenon has not been established. However, high-level expression is due to a difference between human and porcine B domain-deleted factor VIII in translated sequence because the P/OL and HSQ expression cassettes do not contain endogenous factor VIII 5′-UTR sequence, while both possess the first 749 nt (of 1805 nt) of the human factor VIII 3′UTR. Furthermore, the effect occurs at the post-transcriptional level, because there is no difference in P/OL and HSQ mRNA levels in BHK-M cells (Doering et al. (2002) J. Biol. Chem. 277:38345-38349).

TABLE II
Previous Reports of FACTOR VIII Expression.

| FACTOR VIII | FVIII | | | | Cell | |
|---|---|---|---|---|---|---|
| Construct | Level | Assay | Serum | vWf | Line | Reference |
| Human, full | 0.07^{a} | Coatest | + | − | BHK | Wood et al. (1984) |
| length | | | | | | Nature 312: 330- |
| | | | | | | 337 |
| Human, full | 0.16^{a} | Coatest | + | − | COS | Toole et al. |
| length | 0.33^{a} | Coagulation | | | | (1986) Proc. Natl. |
| | | | | | | Acad. Sci. U.S.A. |
| | | | | | | 83: 5939-5942 |
| Human, B | 0.34^{a} | Coatest | − | − | CHO^{c} | Kaufman et al. |
| domain-deleted | | | | | | (1988) |
| | | | | | | J. Biol. Chem. |
| | | | | | | 263: 6352-6362 |
| Human, full | 1.4^{b} | Coatest | − | + | CHO | Kaufman et al. |
| length | | | | | | (1989) Mol. Cell |
| | | | | | | Biol. 9: 1233-1242 |
| Human, B | 5^{a} | Coatest | − | + | CHO | Pittman et al. |
| domain-deleted | | | | | | (1993) Blood |
| | | | | | | 81: 2925-2935 |
| Human, B | 1.5^{a} | Coatest | − | − | CHO | Lind et al (1995) |
| domain-deleted | | | | | | Eur. J. Biochem. |
| | | | | | | 232: 19-27 |
| Human, B | 2.5^{b} | Coagulation | + | − | CHO | Plantier et al. |
| domain-deleted | | | | | | (2001) Thromb. |
| | | | | | | Haemost. 86: 596- |
| | | | | | | 603 |
| Human, B | 3.1^{a} | Coagulation | − | − | BHK | Deering et al. |
| domain-deleted | 10^{b} | | | | | (2002) |
| | | | | | | J. Biol. Chem. 277, |
| | | | | | | 38345-38349 |
| Porcine, B | 41^{a} | Coagulation | − | − | BHK | Deering et al. |
| domain-deleted | 140^{b} | | | | | (2002) |
| | | | | | | J. Biol. Chem. 277, |
| | | | | | | 38345-38349 |

^{a}units/milliliter/24 hours
^{b}units/10⁶ cells/24 hours
^{c}Chinese hamster ovary

### Therapeutics

The nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) encoding a FVIII protein, or variant thereof, can be included in a vector (such as a viral vector) for expression in a cell or a subject.

The nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) encoding a FVIII protein are useful in production of vectors (such as viral vectors disclosed herein), and are also useful in antisense delivery vectors, gene therapy vectors, or vaccine vectors. In certain embodiments, the disclosure provides for gene delivery vectors, and host cells which contain the nucleic acid sequences disclosed herein. In some embodiments, the selected vector may be delivered to a subject by any suitable method, including intravenous injection, ex-vivo transduction, transfection, electroporation, liposome delivery, membrane fusion techniques, high velocity DNA-coated pellets, viral infection, or protoplast fusion, to introduce a transgene into the subject.

In certain embodiments, the disclosure relates to virus particle, e.g., capsids, containing the nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) encoding a FVIII protein disclosed herein. The virus particles, capsids, and recombinant vectors are useful in delivery of the nucleic acid sequences encoding the FVIII proteins to a target cell. The nucleic acids may be readily utilized in a variety of vector systems, capsids, and host cells.

In certain embodiments, nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) may be a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon.

In certain embodiments, a vector may be a lentivirus based (containing lentiviral genes or sequences) vector, e.g., having nucleic acid sequences derived from VSVG or GP64 pseudotypes or both. In certain embodiments, the nucleic acid sequences derived from VSVG or GP64 pseudotypes may be at least one or two or more genes or gene fragments of more than 1000, 500, 400, 300, 200, 100, 50, or 25 continuous nucleotides or nucleotides sequences with greater than 50, 60, 70, 80, 90, 95 or 99% identity to the gene or fragment.

In some embodiments, the nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) disclosed herein are useful in production of AAV vectors. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21 (4): 583-593, 2008).

The left ORF of AAV contains the Rep gene, which encodes four proteins-Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21 (4): 583-593, 2008).

AAV vectors typically contain a transgene expression cassette between the ITRs that replaces the rep and cap genes. Vector particles are produced by the co-transfection of cells with a plasmid containing the vector genome and a packaging/helper construct that expresses the rep and cap proteins in trans. During infection, AAV vector genomes enter the cell nucleus and can persist in multiple molecular states. One common outcome is the conversion of the AAV genome to a double-stranded circular episome by second-strand synthesis or complementary strand pairing.

In the context of AAV vectors, the disclosed vectors typically have a recombinant genome comprising the following structure:
(5′AAV ITR)-(promoter)-(transgene)-(3′AAV ITR)

As discussed above, these recombinant AAV vectors contain a transgene expression cassette between the ITRs that replaces the rep and cap genes. Vector particles are produced, for example, by the co-transfection of cells with a plasmid containing the recombinant vector genome and a packaging/helper construct that expresses the rep and cap proteins in trans.

The transgene can be flanked by regulatory sequences such as a 5′ Kozak sequence and/or a 3′ polyadenylation signal.

The AAV ITRs, and other selected AAV components described herein, may be readily selected from among any AAV serotype, including, without limitation, AAV1, AAV2, AAV3, AAV4, AAV5, AAV6, AAV7, AAV8, AAV9 and function variants thereof. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

In some embodiments, the recombinant AAV vector genome can have a liver-specific promoter, such as any one of the HCB, HSh-HCB, 5′HSh-HCB, 3′HSh-HCB, ABP-HP1-God-TSS, HSh-SynO-TSS, or sHS-SynO-TSS promoters set forth in WO 2016/168728, which is incorporated by reference herein in its entirety.

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev 21 (4): 583-593, 2008).

Methods for producing rAAV suitable for gene therapy are known (see, for example, U.S. Patent Application Nos. 2012/0100606; 2012/0135515; 2011/0229971; and 2013/0072548; and Ghosh et al., Gene Ther 13 (4): 321-329, 2006), and can be utilized with the recombinant nucleic acid molecules and methods disclosed herein.

In some embodiments, the nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) disclosed herein are part of an expression cassette or transgene. See e.g., US Pat. App. Pub. 20150139953. The expression cassette is composed of a transgene and regulatory sequences, e.g., promotor and 5′ and 3′ AAV inverted terminal repeats (ITRs). In one desirable embodiment, the ITRs of AAV serotype 2 or 8 are used. However, ITRs from other suitable serotypes may be selected. An expression cassette is typically packaged into a capsid protein and delivered to a selected host cell.

In some embodiments, the disclosure provides for a method of generating a recombinant adeno-associated virus (AAV) having an AAV serotype capsid, or a portion thereof. Such a method involves culturing a host cell which contains a nucleic acid sequence encoding an adeno-associated virus (AAV) serotype capsid protein; a functional rep gene; an expression cassette composed of AAV inverted terminal repeats (ITRs) and a transgene; and sufficient helper functions to permit packaging of the expression cassette into the AAV capsid protein. See e.g., US Pat. App. Pub. 20150139953.

The components for culturing in the host cell to package an AAV expression cassette in an AAV capsid may be provided to the host cell in trans. Alternatively, any one or more of the components (e.g., expression cassette, rep sequences, cap sequences, and/or helper functions) may be provided by a stable host cell which has been engineered to contain one or more of the required components using methods known to those of skill in the art. Most suitably, such a stable host cell will contain the component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

In some embodiments, the disclosure relates to recombinant vectors comprising a nucleic acids encoding any of the above discussed recombinant amino acid molecules ET3 NoCo (SEQ ID NO: 53), ET3 LCO (SEQ ID NO: 54), ET3 MCO (SEQ ID NO: 55), ET3-30x (SEQ ID NO: 56) in operable combination with transgene. The transgene is a nucleic acid sequence, heterologous to the vector sequences flanking the transgene, which encodes a novel FVIII protein as disclosed herein, and optionally one or more additional proteins of interest. The nucleic acid coding sequence is operatively linked to regulatory components in a manner which permits transgene transcription, translation, and/or expression in a host cell.

The expression cassette can be carried on any suitable vector, e.g., a plasmid, which is delivered to a host cell. The plasmids useful in this disclosure may be engineered such that they are suitable for replication and, optionally, integration in prokaryotic cells, mammalian cells, or both. These plasmids contain sequences permitting replication of the expression cassette in eukaryotes and/or prokaryotes and selection markers for these systems. Preferably, the molecule carrying the expression cassette is transfected into the cell, where it may exist transiently. Alternatively, the expression cassette may be stably integrated into the genome of the host cell, either chromosomally or as an episome. In certain embodiments, the expression cassette may be present in multiple copies, optionally in head-to-head, head-to-tail, or tail-to-tail concatamers. Suitable transfection techniques are known and may readily be utilized to deliver the expression cassette to the host cell.

Generally, when delivering the vector comprising the expression cassette by transfection, the vector and the relative amounts of vector DNA to host cells may be adjusted, taking into consideration such factors as the selected vector, the delivery method and the host cells selected. In addition to the expression cassette, the host cell contains the sequences which drive expression of the AAV capsid protein in the host cell and rep sequences of the same serotype as the serotype of the AAV ITRs found in the expression cassette, or a cross-complementing serotype. Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. Lentiviral versions are discussed at length above.

For AAV, the packaging host cell also typically contains helper functions in order to package the rAAV of the disclosure. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). The desired helper functions, can be provided using any means that allows their expression in a cell.

Introduction into the host cell of any of the vectors disclosed herein may be achieved by any means known in the art or as disclosed above, including transfection, infection, electroporation, liposome delivery, membrane fusion techniques, high velocity DNA-coated pellets, viral infection, cell transfer and protoplast fusion, among others. One or more of the viral vector genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously added factors, for example.

The viral vector techniques disclosed herein can be adapted for use in these and other viral vector systems for in vitro, ex vivo or in vivo gene delivery. The in certain embodiments the disclosure contemplates the use of nucleic acids and vectors disclosed herein in a variety of rAAV and non-rAAV vector systems. Such vectors systems may include, e.g., lentiviruses, retroviruses, poxviruses, vaccinia viruses, and adenoviral systems, among others.

In some embodiments, it is contemplated that viral particles, nucleic acids and vectors disclosed herein are useful for a variety of purposes, including for delivery of therapeutic molecules for gene expression of therapeutic proteins.

Therapeutic proteins encoded by the nucleic acids (e.g., operably in combination with promoters) reported herein include those used for treatment of clotting disorders, including hemophilia A (e.g., using a fVIII protein as provided herein).

In some embodiments, a method of inducing blood clotting in a subject in need thereof is provided. The method comprises administering to the subject a therapeutically effective amount of a vector (such as an AAV vector, a lentiviral vector, or a retroviral vector) encoding a nucleic acid sequences encoding nucleic ET3 NoCo (SEQ ID NO: 53), ET3 LCO (SEQ ID NO: 54), ET3 MCO (SEQ ID NO: 55), ET3-30x (SEQ ID NO: 56) FVIII proteins as described herein. In some embodiments, the subject is a subject with a clotting disorder, such as hemophilia A. In some embodiments, the clotting disorder is hemophilia A and the subject is administered a vector comprising a nucleic acid molecule encoding a protein with FVIII activity.

A treatment option for a patient diagnosed with hemophilia A is the exogenous administration of recombinant FVIII sometimes referred to as FVIII replacement therapy. In some embodiments, a patient with hemophilia A or of a recombinant fVIII protein as described herein. In some patients, these therapies can lead to the development of antibodies that bind to the administered clotting factor. Subsequently, the clotting factor-antibody bound conjugates, typically referred to as inhibitors, interfere with or retard the ability of the exogenous clotting factor to cause blood clotting. Inhibitory autoantibodies also sometimes occur spontaneously in a subject that is not genetically at risk of having a clotting disorder such as hemophilia, termed acquired hemophilia. Inhibitory antibodies assays are typically performed prior to exogenous clotting factor treatment in order to determine whether the anti-coagulant therapy will be effective.

A “Bethesda assay” has historically been used to quantitate the inhibitory strength the concentration of fVIII binding antibodies. In the assay, serial dilutions of plasma from a patient, e.g., prior to having surgery, are prepared and each dilution is mixed with an equal volume of normal plasma as a source of fVIII. After incubating for a couple hours, the activities of fVIII in each of the diluted mixtures are measured. Having antibody inhibitor concentrations that prevent fVIII clotting activity after multiple repeated dilutions indicates a heightened risk of uncontrolled bleeding. Patients with inhibitor titers after about ten dilutions are felt to be unlikely to respond to exogenous fVIII infusions to stop bleeding. A Bethesda titer is defined as the reciprocal of the dilution that results in 50% inhibition of FVIII activity present in normal human plasma. A Bethesda titer greater than 10 is considered the threshold of response to FVIII replacement therapy.

In certain embodiments, the disclosure relates to methods of inducing blood clotting comprising administering an effective amount of a viral particle or capsid comprising a vector comprising a nucleic acid encoding a blood clotting factor as disclosed herein to a subject in need thereof.

In certain embodiments, the subject is diagnosed with hemophilia A or acquired hemophilia or unlikely to respond to exogenous clotting factor infusions (e.g., based on a Bethesda assay result).

In some embodiments, this disclosure relates to methods of gene transfer for the treatment of hemophilia A using an viral vector encoding human FVIII as the gene delivery vehicle. While several such viral based gene therapies for hemophilia A have entered into human clinical trials, they have been hampered by low expression of the therapeutic protein, clotting FVIII, after administration of the virus resulting on only partial correction of the disease. Viral vector toxicity limits the dose of the virus that may be safely administered. Typically, the vector provides efficacious expression of FVIII at viral doses below the threshold of toxicity.

In some embodiments, this disclosure relates to methods of gene transfer for the treatment of hemophilia A using a lentiviral vector encoding human FVIII as the gene delivery vehicle. Delivery of the lentiviral vector encoding the transgene can be, for example, by direct administration to the subject, or by ex vivo transduction and transplantation of hematopoietic stem and progenitor cells with the vector. Typically, the vector provides efficacious expression of FVIII at viral doses below the threshold of toxicity.

In some embodiments, recombinant virus particles, capsids, or vectors comprising nucleic acids disclosed herein can be delivered to liver via the hepatic artery, the portal vein, or intravenously to yield therapeutic levels of therapeutic proteins or clotting factors in the blood. The capsid or vector is preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, sesame oil, and water.

Optionally, the compositions of the disclosure may contain other pharmaceutically acceptable excipients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The recombinant virus particles, capsids, or vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intravenous, intramuscular, subcutaneous, intradermal, and other parental routes of administration. Routes of administration may be combined, if desired.

Dosages of the recombinant virus particles, capsids, or vectors will depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes virus vector.

Recombinant viral vectors of the disclosure provide an efficient gene transfer vehicle which can deliver a selected protein to a selected host cell in vivo or ex vivo even where the organism has neutralizing antibodies to the protein. In one embodiment, the vectors disclosed herein and the cells are mixed ex vivo; the infected cells are cultured using conventional methodologies; and the transduced cells are re-infused into the patient.

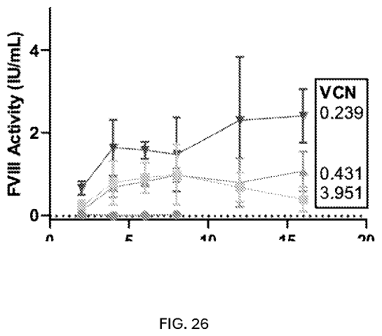

FIG. 26 provides data showing the testing of the disclosed LentET system expressing fVIII. In that figure, the expression codon optimized fVIII nucleic acid sequences, and specifically, the monocyte-directed expression fVIII (SEQ ID NO: 30). In this experiment, HA mice (n=3-5 per cohort) were administered Sca-1+ cells transduced with LV-FVIII vectors. Plasma FVIII activity was determined by chromogenic assay as described above. Blue lines and symbols represent animals receiving ECOM-ET3, green represents ECOL-ET3 and yellow represents NCO-ET3. Red lines and symbols represent untreated control HA mice. Mean VCN of each cohort at 16 weeks is shown in the box. CD68-ECOM-ET3-LV demonstrates a FVIII activity (IU/mL) per vector copy number (VCN) ratio of 40.2 at 16 weeks post gene therapy, while the original non-codon optimized CD68-ET3-LV vector is demonstrating a FVIII: VCN ratio of 0.1 (400× differential)

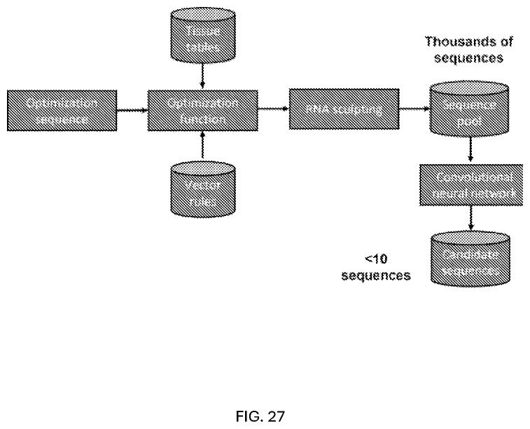

FIG. 27 provides a schematic of the optimization process used to optimize the elements of the system disclosed herein. Specifically, the ECO or expression codon optimization system takes as input the optimization sequence, the tissue for gene expression, and the vector by which the gene will be delivered. These inputs are fed into an optimization algorithm which generates thousands of candidate gene sequences, from which the top candidates are selected.

The present invention has been described above with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

We claim all such modifications and variations that fall within the scope and spirit of the claims below.

1. An expression vector comprising:
a. a lentiviral packaging system comprising,
i. a first plasmid comprising a structural gene selected from a GAG gene, a POL gene, or both GAG and POL genes operatively linked to a first promoter;
ii. a second plasmid comprising an ENV gene encoded by SEQ ID NO: 19 operatively linked to a second promoter;
iii. a third plasmid comprising a REV gene encoded by SEQ ID NO: 36 operatively linked to a third promoter; and
b. a lentiviral transfer plasmid; comprising a transgene operatively linked to a fourth promoter;
c. wherein each of the first promoter, the second promoter, the third promoter, and the fourth promoter are unique one from the other.

2. The expression vector of claim 1, the first plasmid comprising a promoter, a 5′ UTR, a nucleic acid encoding a fVIII variant, 3′ UTR, and a WPRE encoded by SEQ ID NO: 60.

3. The expression vector of claim 1, where the second plasmid is encoded by SEQ ID NO: 42.

4. The expression vector of claim 1, where the third plasmid is encoded by SEQ ID NO: SEQ ID NO: 41.

5. The expression vector of claim 1, where the transfer plasmid is encoded by SEQ ID NO: 63.

6. The expression vector of claim 1, where the transgene is a fVIII encoded by SEQ ID NO: 56.

7. The expression vector of claim 1, the first plasmid encoded by SEQ ID NO: 43.

8. A pharmaceutical composition comprising an an expression vector comprising:
a. a lentiviral packaging system comprising,
i. a first plasmid comprising a structural gene selected from a GAG gene, a POL gene, or both GAG and POL genes operatively linked to a first promoter;
ii. a second plasmid comprising an ENV gene encoded by SEQ ID NO: 19 operatively linked to a second promoter;
iii. a third plasmid comprising a REV gene encoded by SEQ ID NO: 36 operatively linked to a third promoter; and
b. a lentiviral transfer plasmid; comprising a transgene operatively linked to a fourth promoter;
wherein each of the first promoter, the second promoter, the third promoter, and the fourth promoter are unique one from the other.

9. The pharmaceutical composition of claim 8, the first plasmid comprising a promoter, a 5′ UTR, a nucleic acid encoding a fVIII variant, 3′ UTR, and a WPRE encoded by SEQ ID NO: 60.

10. The pharmaceutical composition of claim 8, where the second plasmid is encoded by SEQ ID NO: 42.

11. The pharmaceutical composition of claim 8, where the third plasmid is encoded by SEQ ID NO: SEQ ID NO: 41.

12. The pharmaceutical composition of claim 8, where the transfer plasmid is encoded by SEQ ID NO: 63.

13. The pharmaceutical composition of claim 8, where the transgene is a fVIII encoded by SEQ ID NO: 56.

14. The pharmaceutical composition of claim 8, the first plasmid encoded by SEQ ID NO: 43.

15. A method of inducing blood clotting comprising administering an effective amount of a pharmaceutical composition, the pharmaceutical composition comprising an an expression vector comprising:
a. a lentiviral packaging system comprising,
i. a first plasmid comprising a structural gene selected from a GAG gene, a POL gene, or both GAG and POL genes operatively linked to a first promoter;
ii. a second plasmid comprising an ENV gene encoded by SEQ ID NO: 19 operatively linked to a second promoter;
iii. a third plasmid comprising a REV gene encoded by SEQ ID NO: 36 operatively linked to a third promoter; and
b. a lentiviral transfer plasmid; comprising a transgene operatively linked to a fourth promoter;
wherein each of the first promoter, the second promoter, the third promoter, and the fourth promoter are unique one from the other

16. The method of inducing blood clotting comprising administering an effective amount of a pharmaceutical composition of claim 15, the pharmaceutical composition further comprising, the first plasmid comprising a promoter, a 5′ UTR, a nucleic acid encoding a fVIII variant, 3′ UTR, and a WPRE encoded by SEQ ID NO: 60.

17. The method of inducing blood clotting comprising administering an effective amount of a pharmaceutical composition of claim 15, the pharmaceutical composition further comprising, where the second plasmid is encoded by SEQ ID NO: 42.

18. The method of inducing blood clotting comprising administering an effective amount of a pharmaceutical composition of claim 15, the pharmaceutical composition further comprising, where the third plasmid is encoded by SEQ ID NO: SEQ ID NO: 41.

19. The method of inducing blood clotting comprising administering an effective amount of a pharmaceutical composition of claim 15, the pharmaceutical composition further comprising, where the transfer plasmid is encoded by SEQ ID NO: 63.

20. The method of inducing blood clotting comprising administering an effective amount of a pharmaceutical composition of claim 15, the pharmaceutical composition further comprising, where the transgene is a fVIII encoded by SEQ ID NO: 56.
